(19) United States
(12) Patent Application Publication
(10) Pub. No.: US 2015/0368313 A1
(43) Pub. Date: Dec. 24, 2015

(54) Novel Glucagon Analogues

(71) Applicant: Novo Nordisk A/S, Bagsvaerd (DK)

(72) Inventors: Jesper F. Lau, Farum (DK); Thomas Kruse, Herlev (DK); Henning Thoegersen, Farum (DK); Thomas N. Krogh, Jyllinge (DK); Ulrich Sensfuss, Koebenhavn (DK)

(21) Appl. No.: 14/814,686

(22) Filed: Jul. 31, 2015

Related U.S. Application Data
(63) Continuation of application No. 13/984,185, filed on Oct. 18, 2013.
(60) Provisional application No. 61/468,285, filed on Mar. 28, 2011.
(60) Provisional application No. 61/539,128, filed on Sep. 26, 2011.

(30) Foreign Application Priority Data
Mar. 28, 2011 (EP) 11159967.6
Sep. 23, 2011 (EP) 11182475.1

(51) Int. Cl. C07K 14/605; A61K 38/26

(52) U.S. Cl. CPC C07K 14/605; A61K 38/26

(57) ABSTRACT

The present invention relates to novel glucagon peptides with improved stability and solubility at neutral pH, to the use of the compounds in therapy, to methods of treatment comprising administration of the compounds to patients in need thereof, and to the use of the compounds in the manufacture of medicaments. The glucagon peptides of the invention are of particular interest in relation to the treatment of hyperglycemia, diabetes and obesity, as well as a variety of diseases or conditions associated with hyperglycemia, diabetes and obesity.

### CROSS-REFERENCE TO RELATED APPLICATIONS

This application is a continuation of U.S. application Ser. No. 13/984,185, filed Aug. 7, 2013 which is a 35 U.S.C. §371 National Stage application of International Application PCT/EP2012/055481 (WO 2012/130866 A1), filed Mar. 28, 2012, which claims priority to European Patent Application 11159967.6, filed Mar. 28, 2011 and European Patent Application 11182475.1, filed Sep. 23, 2011; this application claims priority under 35 U.S.C. §119 to U.S. Provisional Application 61/468,285; filed Mar. 28, 2011 and U.S. Provisional Application 61/539,128, filed Sep. 26, 2011; the contents of which are incorporated herein by reference.

### FIELD OF THE INVENTION

The present invention relates to novel derivatives of glucagon peptide analogues with improved physical stability and solubility, to the use of said peptides in therapy, to methods of treatment comprising administration of said peptides to patients, and to the use of said peptides in the manufacture of medicaments.

### INCORPORATION-BY-REFERENCE OF THE SEQUENCE LISTING

In accordance with 37 C.F.R. §1.52(e)(5), Applicants enclose herewith the Sequence Listing for the above-captioned application entitled “SeqList_{—}8330 US04”, created on Jul. 24, 2015. The Sequence Listing is made up of 494 bytes, and the information contained in the attached “SeqList_{—}8330 US04” is identical to the information in the specification as originally filed. No new matter is added.

### BACKGROUND OF THE INVENTION

The precise control of blood glucose levels is of vital importance to humans as well as other mammals. It is well established that the two hormones insulin and glucagon are important for maintenance of correct blood glucose levels. While insulin acts in the liver and peripheral tissues by reducing blood glucose levels via increased peripheral uptake of glucose and reduced glucose output from the liver, glucagon acts mainly on the pancreas and liver, by increasing blood glucose levels via up-regulation of gluconeogenesis and glycogenolysis. Glucagon has also been reported to increase lipolysis, to induce ketosis and to reduce plasma triglyceride levels in plasma [Schade and Eaton, Acta Diabetologica, 1977, 14, 62].

Human glucagon is a linear peptide 29 residues long and the distinctive combination of size and sequence of glucagon leads to considerable difficulties in handling the peptide in manufacture and in use. The molecule is too small to engage in productive and stabilizing tertiary structures, yet it is big enough to engage in phase transitions e.g to form beta-sheet like aggregates or fibrillar structures. Human glucagon has inherently low solubility in the pH 3-9 range and a choice must be made between acidic and basic formulations. In addition, due to the presence of several residues in native glucagon that are prone to base-catalyzed deamidation, glucagon can only be handled for a short time at high pH (>10). Thus, the problem of handling glucagon in solution arises from rather small energy barriers separating the completely random conformation from the more distinctive structures including e.g. those necessary for binding and activation of the receptor and those capable of forming fibrils. The commercial glucagon (Eli Lilly and Novo Nordisk) primarily used for insulin-induced hypoglycemia (e.g. insulin shock) is supplied as a freeze-dried solid which must be dissolved prior to use. As a result from glucagon's instability in aqueous solutions, becoming viscous or turbid after a few hours, the solution must be injected shortly after preparation. The complex and time consuming dissolution process is considered a major problem for patients or relatives who may need to act quickly to counteract the hypoglycemia.

In addition to the well-known use of glucagon for the treatment of acute hypoglycemia the most important application is based on its spasmolytic effect on smooth muscles which is used clinically in connection with several imaging procedures, especially X-ray of the abdominal region.

Several patent applications disclosing different glucagon-based analogues and GLP-1/glucagon receptor co-agonists are known in the art, such as e.g. patents WO2008/086086, WO2008/101017, WO2007/056362, WO2008/152403, WO96/29342, WO09/155257, WO10/011439 and WO10/148089. Some of the GLP-1/glucagon receptor co-agonists disclosed in these patents refer to specific mutations relative to native human glucagon. Other glucagon analogs disclosed are PEGylated (e.g. WO2007/056362) or acylated in specific positions of native human glucagon (e.g. WO96/29342). Glucagon for prevention of hypoglycaemia has been disclosed, as e.g. in patent application U.S. Pat. No. 7,314,859.

The peptides of the present invention provide novel derivative of glucagon peptide analogues with improved physical stability in solution.

### SUMMARY OF THE INVENTION

The present invention relates to novel derivatives of glucagon peptide analogues with improved physical stability in solution and improved solubility at neutral pH, to the use of said peptides in therapy, to methods of treatment comprising administration of said peptides to patients, and to the use of said peptides in the manufacture of medicaments for use in the treatment of diabetes, obesity and related diseases and conditions, such as hypoglycemia.

In a first embodiment (embodiment 1), the present invention relates to a derivative of glucagon peptide analogue of formula [I]:

His-X₂-X₃-Gly-Thr-Phe-Thr-Ser-Asp-X₁₀-Ser-X₁₂-Tyr-Leu-X₁₅-X₁₆-Arg-X₁₈-Ala-X₂₀-X₂₁-Phe-Val-X₂₄-Trp-Leu-X₂₇-X₂₈-X₂₉-X₃₀ [I]

comprising a substituent attached to the nitrogen of the side chain of an amino acid in positions X₁₂, X₁₆, X₂₀, X₂₁, X₂₄, X₂₈, X₂₉, and/or X₃₀ of said glucagon peptide and wherein said substituent has the formula II:

Y₁-Y₂-Y₃-Y₄-Y₅-Y₆-Y₇-Y₈-Y₉-Y₁₀-Y₁₁-Y₁₂ [II]

wherein

Y₁, Y₂, Y₃, Y₄, Y₅, Y₆, Y₇, Y₈, Y₉, Y₁₀ and Y₁₁ is individually absent or individually represents an amino acid or i, ii, iii or iv, which have the stereochemistry L or D or the structure v

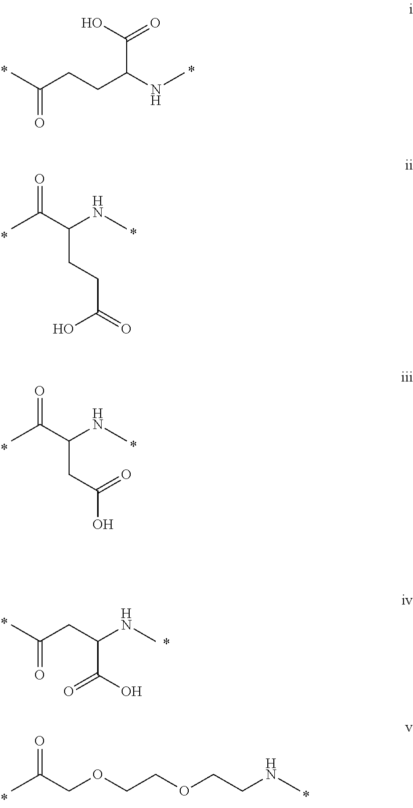

and,

Y₁₂ is absent or represents a C₂₋₆ acyl group or a succinoyl moiety provided that the substituent of formula II contains between three and ten negatively charged moieties, or a pharmaceutically acceptable salt, amide or carboxylic acid thereof.

In another embodiment (embodiment 2), the present invention relates to a derivative of glucagon peptide analogue according to embodiment 1, wherein:

Y₁ is absent or represents an amino acid, such as but not limited to Arg, ε-Lys or Gly;

Y₂, Y₃, Y₄, Y₅, Y₆, Y₇, Y₈, Y₉, Y₁₀ or Y₁₁ is individually absent or individually represents an amino acid or i or ii;

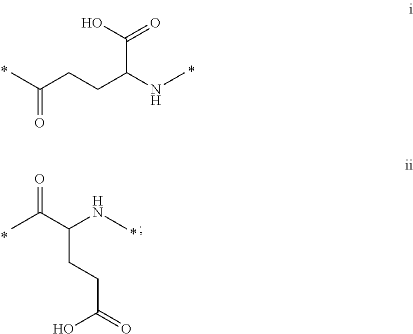

and

Y₁₂ is absent or represents a structure of the formula vi, vii, viii, ix, x or xi:

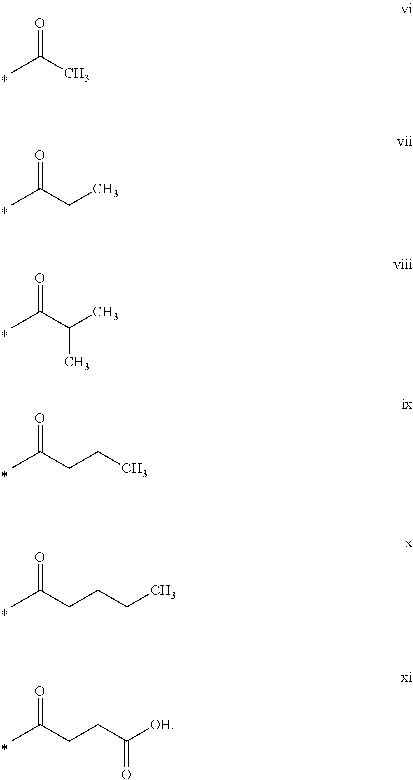

In another embodiment (embodiment 3), the present invention relates to a derivative of glucagon peptide analogue according to embodiment 1, wherein:

Y₁ is absent or represents an amino acid, such as but not limited to Arg, 8-Lys or Gly;

Y₂, Y₃, Y₄, Y₅, Y₆, Y₇, Y₈, Y₉, Y₁₀ or Y₁₁ is individually absent or individually represents i or ii;

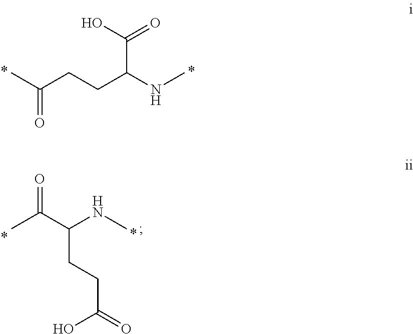

and

Y₁₂ is absent or represents a structure of the formula vi, vii, viii, ix, x or xi:

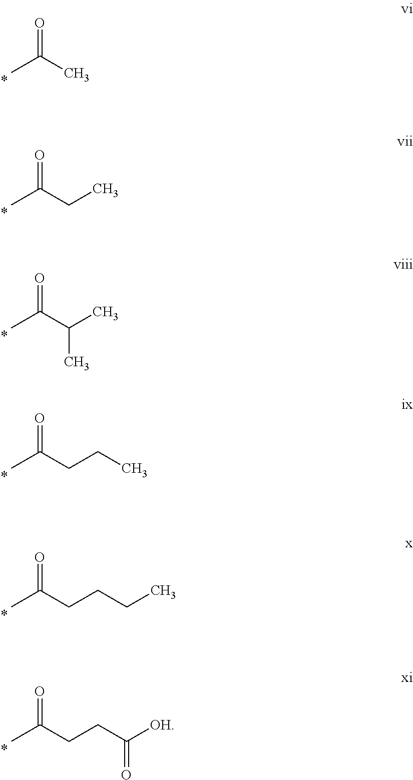

In another embodiment (embodiment 3A), the present invention relates to a derivative of glucagon peptide analogue according to embodiment 1, wherein:

Y₁ is absent or represents Arg, ε-Lys or Gly;

Y₂, Y₃, Y₄, Y₅, Y₆, Y₇, Y₈, Y₉, Y₁₀ or Y₁₁ is individually absent or individually represents i or ii;

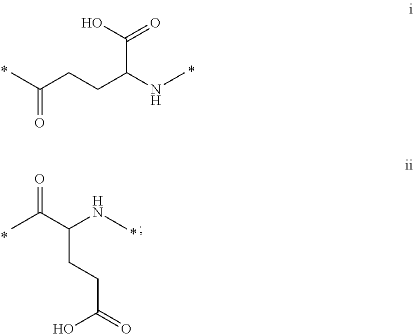

and

Y₁₂ is absent or represents a structure of the formula vi, vii, viii, ix, x or xi:

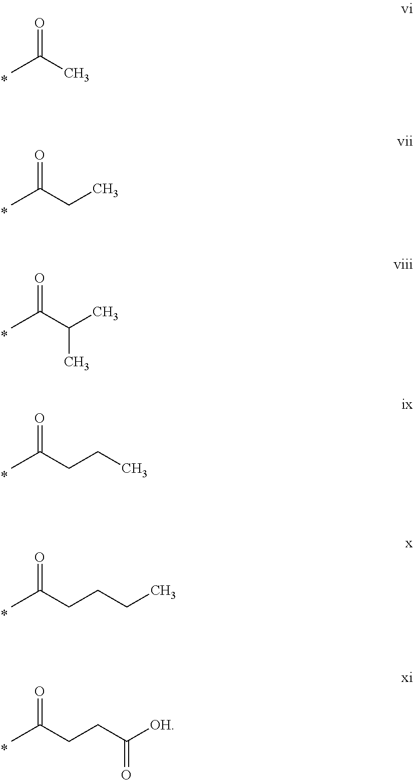

The derivative of glucagon peptide analogues of the present invention enable liquid formulation with long term stability and comprise between three to ten negatively charged moieties/groups attached to a side chain. Such glucagon peptides enable liquid formulation in a pen system, which is much more convenient and easy to use, being an advantage in relation to present commercially available glucagon GlucaGen® HypoKit.

The present invention further relates to the use of the derivative of glucagon peptide analogues of the present invention in therapy, to pharmaceutical compositions comprising compounds of the invention and the use of the compounds of the invention in the manufacture of medicaments.

### DESCRIPTION OF THE DRAWINGS

FIG. 1 shows solubility and lag time of example 2 (Assay III).

FIG. 2 shows solubility and lag time of example 3 (Assay III).

FIG. 3 shows solubility and lag time of example 4 (Assay III).

FIG. 4 shows pharmacokinetic (A) and pharmacodynamic (B) profiles after SC dosing of GlucaGen® HypoKit (black, n=5) and example 2 (grey, n=3) to Göttingen minipigs. Data are mean±SEM.

FIG. 5 shows far UV CD spectra of formulated example 5 stored at various conditions.

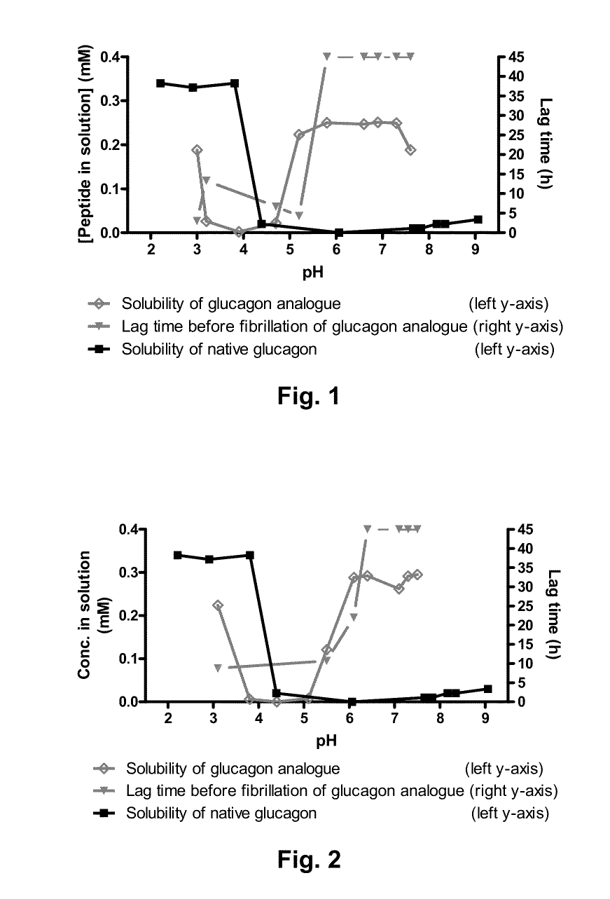

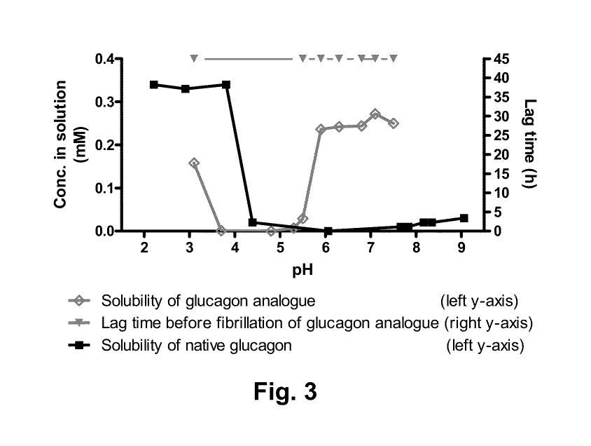

### DESCRIPTION OF THE INVENTION

Among further embodiments of the present invention are the following:
4. The derivative of glucagon peptide analogue according to any of the previous embodiments, wherein said substituent is selected from one of the following structures:

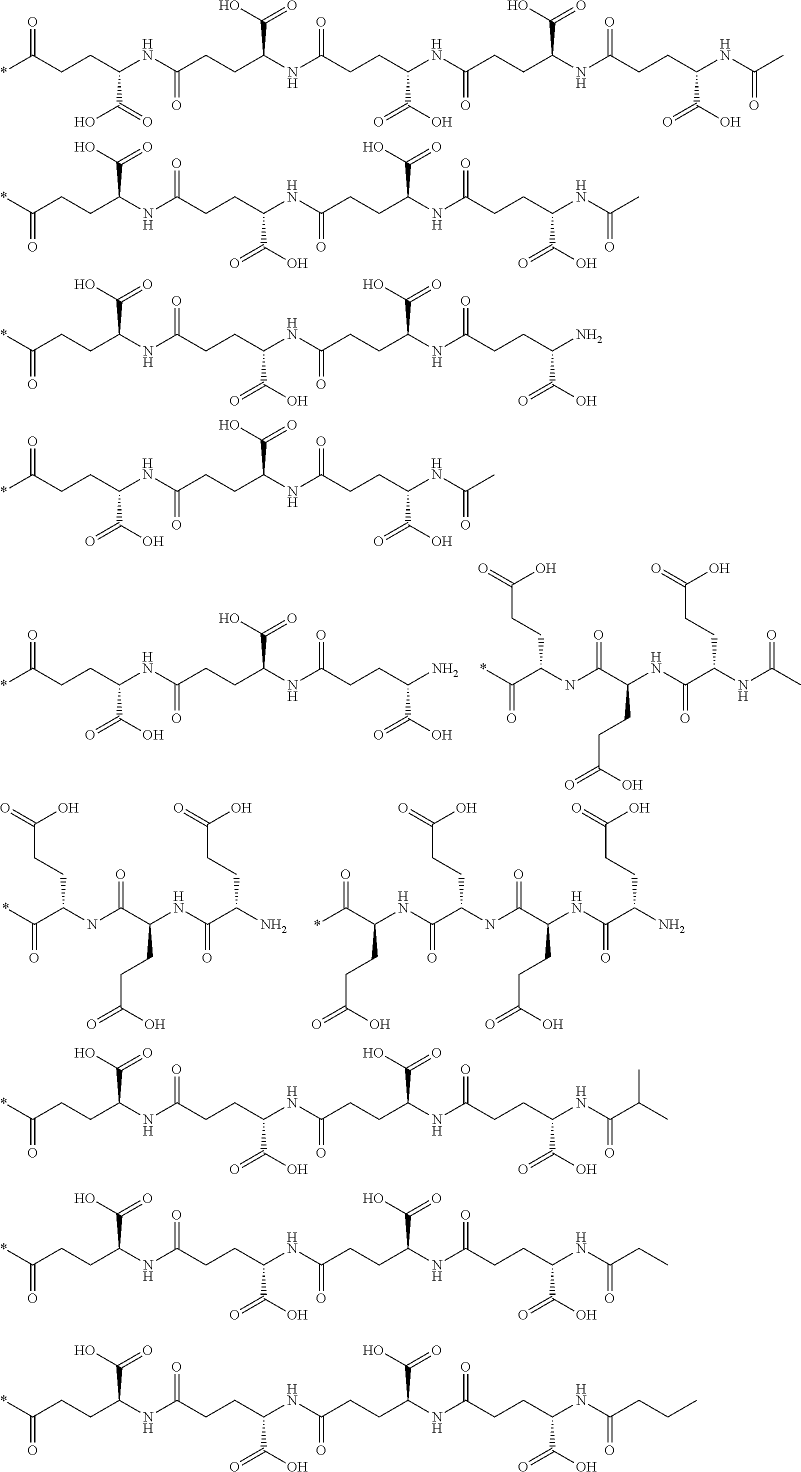

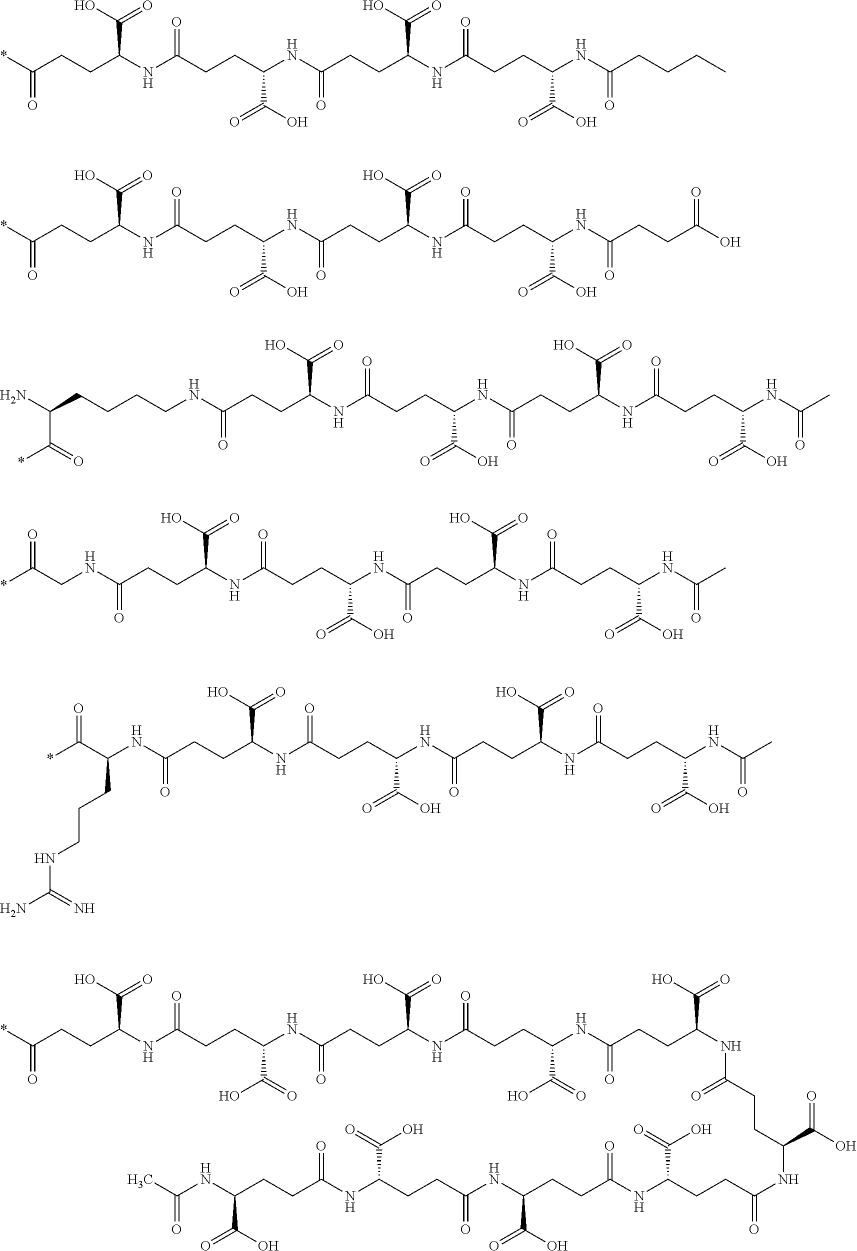

wherein * represents the attachment point to the peptide.
5. A derivative of glucagon peptide analogue according to any of the previous embodiments, wherein said substituent is selected from one of the following structures:

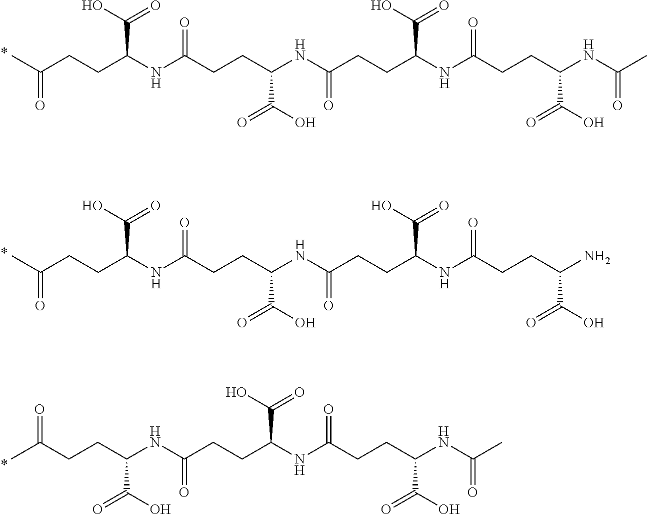

wherein * represents the attachment point to the peptide.
6. The derivative of glucagon peptide analogue according to any one of the previous embodiments, wherein the structures of embodiments 4-5, have the stereochemistry L.
7. The derivative of glucagon peptide analogue according to any one of the previous embodiments, wherein the structures of embodiments 4-5, have the inverted stereochemistry (i.e. stereochemistry D).
8. The derivative of glucagon peptide analogue according to any one of the previous embodiments, wherein said substituent comprises three to ten of said negatively charged moieties.
9. The derivative of glucagon peptide analogue according to embodiment 8, wherein said substituent comprises four to ten of said negatively charged moieties.
10. The derivative of glucagon peptide analogue according to embodiment 8, wherein said substituent comprises three to five of said negatively charged moieties.
11. The derivative of glucagon peptide analogue according to embodiment 8, wherein said substituent comprises four or five of said negatively charged moieties.
12. The derivative of glucagon peptide analogue according to embodiment 8, wherein said substituent comprises three of said negatively charged moieties.
13. The derivative of glucagon peptide analogue according to embodiment 8, wherein said substituent comprises four of said negatively charged moieties.
14. The derivative of glucagon peptide analogue according to embodiment 8, wherein said substituent comprises five of said negatively charged moieties.
15. The derivative of glucagon peptide analogue according to embodiment 8, wherein said substituent comprises ten of said negatively charged moieties.
16. The derivative of glucagon peptide analogue according to any one of the previous embodiments, wherein the terminal negatively charged moiety is N-acylated with a C₂₋₆ acyl group or a succinoyl moiety.
17. The derivative of glucagon peptide analogue according to any one of the previous embodiments, wherein the terminal negatively charged moiety is N-acylated with a C₂₋₆ acyl group.
18. The derivative of glucagon peptide analogue according to any one of the previous embodiments, wherein the terminal negatively charged moiety is N-acylated with a succinoyl moiety.
19. A derivative of glucagon peptide analogue according to any one of the previous embodiments, wherein said negatively charged moieties of said substituent are represented by Glu and/or γGlu and/or Asp and/or βAsp, a carboxylic acid, sulphonic acid or a tetrazole moiety.
20. A derivative of glucagon peptide analogue according to any one of the previous embodiments, wherein said negatively charged moieties of said substituent are represented by Glu and/or γGlu.
21. A derivative of glucagon peptide analogue according to any one of the previous embodiments, wherein said negatively charged moieties of said substituent, are represented by Glu.
22. A derivative of glucagon peptide analogue according to any one of the previous embodiments, wherein said substituent is independently represented by Glu-Glu-Glu, Glu-Glu-Glu-Glu, Glu-Glu-Glu-Glu-Glu or Glu-Glu-Glu-Glu-Glu-Glu-Glu-Glu-Glu-Glu.
23. A derivative of glucagon peptide analogue according to embodiment 22, wherein said substituent is independently represented by Glu-Glu-Glu.
24. A derivative of glucagon peptide analogue according to embodiment 22, wherein said substituent is independently represented by Glu-Glu-Glu-Glu.
25. A derivative of glucagon peptide analogue according to embodiment 22, wherein said substituent is independently represented by Glu-Glu-Glu-Glu-Glu.
26. A derivative of glucagon peptide analogue according to embodiment 22, wherein said substituent is independently represented by Glu-Glu-Glu-Glu-Glu-Glu-Glu-Glu-Glu-Glu.
27. A derivative of glucagon peptide analogue according to embodiment 20, wherein said negatively charged moieties are represented by γGlu moieties.
28. A derivative of glucagon peptide analogue according to any one of the previous embodiments, wherein said substituent is independently represented by γGlu-γGlu-γGlu, γGlu-γGlu-γGlu-γGlu, γGlu-γGlu-γGlu-γGlu-γGlu or γGlu-γGlu-γGlu-γGlu-γGlu-γGlu-γGlu-γGlu-γGlu-γGlu.
29. A derivative of glucagon peptide analogue according to embodiment 28, wherein said substituent is represented by γGlu-γGlu-γGlu.
30. A derivative of glucagon peptide analogue according to embodiment 28, wherein said substituent is represented by γGlu-γGlu-γGlu-γGlu.
31. A derivative of glucagon peptide analogue according to embodiment 28, wherein said substituent is represented by γGlu-γGlu-γGlu-γGlu-γGlu.
32. A derivative of glucagon peptide analogue according to embodiment 28, wherein said substituent is represented by Glu-γGlu-γGlu-γGlu-γGlu-γGlu-γGlu-γGlu-γGlu-γGlu.
33. The derivative of glucagon peptide analogue according to any one of the previous embodiments, wherein said substituent is attached to the side chain of an amino acid in positions X₁₂, X₁₆, X₂₀, X₂₁, X₂₄, X₂₈, X₂₉ or X₃₀ of said glucagon peptide.
34. The derivative of glucagon peptide analogue according to embodiment 33, wherein said substituent is attached to the side chain of an amino acid in positions X₁₂, X₂₀, X₂₄, X₂₈, X₂₉ or X₃₀ of said glucagon peptide.
35. The derivative of glucagon peptide analogue according to embodiment 33, wherein said substituent is attached to the side chain of an amino acid in positions X₁₂ or X₂₄ of said glucagon peptide.
36. The derivative of glucagon peptide analogue according to embodiment 33, wherein said substituent is attached to the side chain of an amino acid in position X₁₂ of said glucagon peptide.
37. The derivative of glucagon peptide analogue according to embodiment 33, wherein said substituent is attached to the side chain of an amino acid in position X₁₆ of said glucagon peptide.
38. The derivative of glucagon peptide analogue according to embodiment 33, wherein said substituent is attached to the side chain of an amino acid in position X₂₀ of said glucagon peptide.
39. The derivative of glucagon peptide analogue according to embodiment 33, wherein said substituent is attached to the side chain of an amino acid in position X₂₁ of said glucagon peptide.
40. The derivative of glucagon peptide analogue according to embodiment 33, wherein said substituent is attached to the side chain of an amino acid in position X₂₄ of said glucagon peptide.
41. The derivative of glucagon peptide analogue according to embodiment 33, wherein said substituent is attached to the side chain of an amino acid in position X₂₈ of said glucagon peptide.
42. The derivative of glucagon peptide analogue according to embodiment 33, wherein said substituent is attached to the side chain of an amino acid in position X₂₉ of said glucagon peptide.
43. The derivative of glucagon peptide analogue according to embodiment 33, wherein said substituent is attached to the side chain of an amino acid in position X₃₀ of said glucagon peptide.
44. The derivative of glucagon peptide analogue according to embodiment 33, wherein said substituent is attached to the side chain of a Lys in position 12, 16, 20, 21, 24, 28, 29 or 30 of said glucagon peptide.
45. The derivative of glucagon peptide analogue according to embodiment 35, wherein said substituent is attached to the side chain of a Lys in position 12 or 24 of said glucagon peptide.
46. The derivative of glucagon peptide analogue according to embodiment 40, wherein said substituent is attached to the side chain of a Lys in position 24 of said glucagon peptide.
47. The derivative of glucagon peptide analogue according to any one of the previous embodiments, wherein said glucagon peptide comprises up to 15 amino acid residue substitutions in said glucagon peptide.
48. The derivative of glucagon peptide analogue according to embodiment 46, wherein said glucagon peptide comprises zero, one, two, three, four, five, six, seven, eight, nine, ten, eleven, twelve, thirteen, fourteen or fifteen amino acid residue substitutions in said glucagon peptide.
49. The derivative of glucagon peptide analogue according to embodiment 46, wherein said glucagon peptide comprises zero, three, four, five, seven or ten amino acid residues substitutions in said glucagon peptide.
50. The derivative of glucagon peptide analogue according to embodiment 46, wherein said glucagon peptide comprises one amino acid residues substitutions in said glucagon peptide.
51. The derivative of glucagon peptide analogue according to embodiment 46, wherein said glucagon peptide comprises two amino acid residues substitutions in said glucagon peptide.
52. The derivative of glucagon peptide analogue according to embodiment 46, wherein said glucagon peptide comprises three amino acid residues substitutions in said glucagon peptide.
53. The derivative of glucagon peptide analogue according to embodiment 46, wherein said glucagon peptide comprises four amino acid residues substitutions in said glucagon peptide.
54. The derivative of glucagon peptide analogue according to embodiment 46, wherein said glucagon peptide comprises five amino acid residues substitutions in said glucagon peptide.
55. The derivative of glucagon peptide analogue according to embodiment 46, wherein said glucagon peptide comprises six amino acid residues substitutions in said glucagon peptide.
56. The derivative of glucagon peptide analogue according to embodiment 46, wherein said glucagon peptide comprises seven amino acid residues substitutions in said glucagon peptide.
57. The derivative of glucagon peptide analogue according to embodiment 46, wherein said glucagon peptide comprises eight amino acid residues substitutions in said glucagon peptide.
58. The derivative of glucagon peptide analogue according to embodiment 46, wherein said glucagon peptide comprises nine amino acid residues substitutions in said glucagon peptide.
59. The derivative of glucagon peptide analogue according to embodiment 46, wherein said glucagon peptide comprises ten amino acid residues substitutions in said glucagon peptide.
60. The derivative of glucagon peptide analogue according to embodiment 46, wherein said glucagon peptide comprises eleven amino acid residues substitutions in said glucagon peptide.
61. The derivative of glucagon peptide analogue according to embodiment 46, wherein said glucagon peptide comprises twelve amino acid residues substitutions in said glucagon peptide.
62. The derivative of glucagon peptide analogue according to embodiment 46, wherein said glucagon peptide comprises thirteen amino acid residues substitutions in said glucagon peptide.
63. The derivative of glucagon peptide analogue according to embodiment 46, wherein said glucagon peptide comprises fourteen amino acid residues substitutions in said glucagon peptide.
64. The derivative of glucagon peptide analogue according to embodiment 46, wherein said glucagon peptide comprises fifteen amino acid residues substitutions in said glucagon peptide.
65. The derivative of glucagon peptide analogue according to any one of the previous embodiments, wherein said glucagon peptide comprises up to 15 amino acid residue substitutions and wherein:
   X₂ represents Ser, Aib, Thr, Ala, or Gly;
   X₃ represents Gln or His;
   X₁₀ represents Tyr or Val;
   X₁₂ represents Lys, Orn or Arg;
   X₁₅ represents Asp or Glu;
   X₁₆ represents Ser, Thr, Lys, Val, Tyr, Phe, Leu, Ile, Trp or Orn;
   X₁₈ represents Arg, Lys, Ala or Orn;
   X₂₀ represents Gln, Lys, Ala, Glu or Orn;
   X₂₁ represents Asp, Glu, Lys or Orn;
   X₂₄ represents Gln, Lys, or Orn;
   X₂₇ represents Met or Leu;
   X₂₈ represents Asn, Lys, Ser or Orn;
   X₂₉ represents Thr, Lys or Orn and
   X₃₀ is absent or represents Lys, Pro or Orn.
66. The derivative of glucagon peptide analogue according to embodiment 65, wherein: X₂ represents Ser, Aib, Thr, Ala or Gly; X₃ represents Gln or His; X₁₀ represents Tyr or Val; X₁₂ represents Lys, Orn or Arg; X₁₅ represents Asp or Glu; X₁₆ represents Ser, Thr, Val, Tyr, Phe, Leu, Ile, Trp, Orn or Lys; X₁₈ represents Arg, Lys, Ala or Orn; X₂₀ represents Gln, Lys, Ala, Glu or Orn; X₂₁ represents Asp, Glu, Lys or Orn; X₂₄ represents Gln, Lys, or Orn; X₂₇ represents Met or Leu; X₂₈ represents Asn, Lys, Ser or Orn; X₂₉ represents Thr, Orn or Lys and X₃₀ is absent or represents Lys, Orn or Pro.
67. The derivative of glucagon peptide analogue according to embodiment 65, wherein X₂ represents Ser, Aib, Thr, Ala or Gly.
68. The derivative of glucagon peptide analogue according to embodiment 65, wherein X₃ represents Gln or His.
69. The derivative of glucagon peptide analogue according to embodiment 65, wherein X₁₀ represents Tyr or Val.
70. The derivative of glucagon peptide analogue according to embodiment 65, wherein X₁₂ represents Lys or Arg.
71. The derivative of glucagon peptide analogue according to embodiment 65, wherein X₁₅ represents Asp or Glu.
72. The derivative of glucagon peptide analogue according to embodiment 65, wherein X₁₆ represents Ser, Thr, Val, Tyr, Phe, Leu, Ile, Trp or Lys.
73. The derivative of glucagon peptide analogue according to embodiment 65, wherein X₁₈ represents Arg or Ala.
74. The derivative of glucagon peptide analogue according to embodiment 65, wherein X₂₀ represents Gln, Lys, Ala or Glu.
75. The derivative of glucagon peptide analogue according to embodiment 65, wherein X₂₁ represents Asp, Glu or Lys.
76. The derivative of glucagon peptide analogue according to embodiment 65, wherein X₂₄ represents Gln or Lys.
77. The derivative of glucagon peptide analogue according to embodiment 65, wherein X₂₇ represents Met or Leu.
78. The derivative of glucagon peptide analogue according to embodiment 65, wherein X₂₈ represents Asn, Ser or Lys.
79. The derivative of glucagon peptide analogue according to embodiment 65, wherein X₂₉ represents Thr or Lys.
80. The derivative of glucagon peptide analogue according to embodiment 65, wherein X₃₀ is absent or represents Lys or Pro.

The present invention relates to novel glucagon analogues with improved solubility and improved physical stability towards gel and fibril formation.

Peptides may undergo various changes of physical state. Peptides may precipitate due to lack of solubility at a certain set of conditions, e.g. due to neutralization of repulsing charges on amino acid side chains due to a change of pH. Another physical change is the formation of amyloid fibrils, which involves a conformational change into β-sheet rich macromolecular fiber structures. Other macromolecular structures may be formed by less systematic structural repeats due to aggregation. In the two latter instances peptide substance may eventually be observed as a precipitate. In fact these physical changes may to some extent be interrelated, e.g. solubility versus pH and fibril formation is related [Schmittschmitt and Scholtz, Protein Science, 12, 10, 2374-2378, 2003]. Furthermore, it is very difficult to distinguish these phenomena by visual inspection only, therefore the result of these changes are often described by the general term “precipitate”.

Other changes of physical state include adsorption to surfaces observed as a loss of content of peptide from solution, and the change from a liquid solution to a gel. Nevertheless, the observation of a precipitate disregardless its nature or formation of a gel is a problem when in a pharmaceutical injectable during its storage and in-use time.

Glucagon has a very low aqueous solubility at neutral pH, which disables pharmaceutical formulation at neutral pH. Even when dissolved at acidic pH, glucagon may undergo various phase transitions that depend on concentration and temperature and is thus very physically unstable. After dissolving samples of glucagon in hydrochloric acid a lag-phase may occur where the viscosity of the sample is low and the solution is fully transparent. After some hours the viscosity begins to increase—indicative of a gelformation (Beaven et al, European J. Biochem. 11 (1969) 37-42). After reaching a plateau viscosity may begin to fall again and at the same time fibrils may appear and precipitate out of solution. The process is seedable, addition of a small amount of pre-formed gel reduce the lag-phase. Formation of gels and fibrillation is highly dependent of physical stress, such as heating and shaking, both increasing the rate of the process.

The inventors surprisingly found that the compounds of the present invention show improved aqueous solubility at neutral pH or slightly basic pH. Furthermore, the present inventors have also surprisingly found that the glucagon analogues of the present invention have improved stability towards formation of gels and fibrils in aqueous solutions. The stability of the compounds of the present invention may be measured by Assay (II) and Assay (III).

In one embodiment, the glucagon analogues of this invention can be co-formulated with GLP-1 analogues or insulin analogues, forming stable pharmaceutical compositions.

Combination of insulin and glucagon therapy may be advantageous compared to insulin-only therapy comes from the architecture of the human defense against hypoglycaemia. Normally, in a postprandial situation when blood glucose levels become low the first hormonal response is reduction in the production of insulin. When blood glucose drop further the second line response is production of glucagon—resulting in increased glucose output from the liver. When diabetics receive an exogenous dose of insulin that is too high the natural response of raised glucagon is prevented by the presence of exogenous insulin, since insulin has an inhibiting effect on glucagon production. Consequently, slight overdosing of insulin may cause hypoglycaemia. Presently, many diabetic patients tend to prefer to use a little less insulin than optimal in fear of hypoglycaemic episodes which may be life-threatening.

The fact that the compounds of the present invention are soluble at neutral pH, may allow a co-formulation with insulin and allow for more stable blood glucose levels and a reduced number of hypoglycaemic episodes, as well as a reduced risk of diabetes related complications.
81. The derivative of glucagon peptide analogue according to any one of the previous embodiments, wherein said glucagon peptide is selected from glucagon (1-29), glucagon (1-29)-amide, or an analogue thereof.
82. The derivative of glucagon peptide analogue according to embodiment 81, wherein said glucagon peptide is glucagon (1-29) or an analogue thereof.
83. The derivative of glucagon peptide analogue according to embodiment 81, wherein said glucagon peptide is glucagon (1-29)-amide or an analogue thereof.
84. A derivative of glucagon peptide analogue according to any one of the previous embodiments, wherein said glucagon peptide comprises C-terminal extensions of up to three amino acid residues.
85. A derivative of glucagon peptide analogue according to embodiment 84, wherein said glucagon peptide comprises C-terminal extensions of up to two amino acid residues.
86. A derivative of glucagon peptide analogue according to embodiment 84, wherein said glucagon peptide comprises C-terminal extensions of one amino acid residue.
87. A derivative of glucagon peptide analogue according to any one the previous embodiments, wherein the glucagon peptide is a C-terminal amide or a C-terminal carboxylic acid.
88. A derivative of glucagon peptide analogue according to embodiment 87, wherein said glucagon peptide is a C-terminal amide.
89. A derivative of glucagon peptide analogue according to embodiment 87, wherein said glucagon peptide is a C-terminal carboxylic acid.
90. The derivative of glucagon peptide analogue according to any one of the previous embodiments, selected from the group consisting of Chem.2, Chem.3, Chem.4, Chem.5, Chem.6, Chem.7, Chem.8, Chem.9, Chem.10, Chem.11, Chem.12, Chem.13, Chem.16, Chem.17, Chem.18, Chem.19, Chem.20, Chem.21, Chem.22, Chem.23, Chem.24, Chem.25, Chem.26, Chem.27, Chem.28, Chem.29, Chem.30, Chem.31, Chem.32, Chem.33, Chem.34, Chem.35, Chem.36, Chem.37, Chem.38, Chem.39, Chem.40, Chem.41, Chem.42, Chem.43, Chem.44, Chem.45, Chem.46, Chem.47 and Chem.48, Chem.49, Chem.50, Chem.51, Chem.52, Chem.53, Chem.54, Chem.55, Chem.56, Chem.57, Chem.58, Chem.59 Chem.60, Chem.61, Chem.62, Chem.63, Chem.64, Chem.65, Chem.66, Chem.67, Chem.68 and Chem.69.

By “simultaneous” dosing of a preparation of a compound of the present invention and a preparation of anti-obesity or anti-diabetic agents is meant administration of the compounds in single-dosage form, or administration of a first agent followed by administration of a second agent with a time separation of no more than 15 minutes, preferably 10, more preferred 5, more preferred 2 minutes. Either factor may be administered first.

By “sequential” dosing is meant administration of a first agent followed by administration of a second agent with a time separation of more than 15 minutes. Either of the two unit dosage form may be administered first. Preferably, both products are injected through the same intravenous access.

As already indicated, in all of the therapeutic methods or indications disclosed above, a compound of the present invention may be administered alone. However, it may also be administered in combination with one or more additional therapeutically active agents, substances or compounds, either sequentially or concomitantly.

A typical dosage of a compound of the invention when employed in a method according to the present invention is in the range of from about 0.001 to about 100 mg/kg body weight per day, preferably from about 0.01 to about 10 mg/kg body weight, more preferably from about 0.01 to about 5 mg/kg body weight per day, e.g. from about 0.05 to about 10 mg/kg body weight per day or from about 0.03 to about 5 mg/kg body weight per day administered in one or more doses, such as from 1 to 3 doses. The exact dosage will depend upon the frequency and mode of administration, the sex, age, weight and general condition of the subject treated, the nature and severity of the condition treated, any concomitant diseases to be treated and other factors evident to those skilled in the art.

Compounds of the invention may conveniently be formulated in unit dosage form using techniques well known to those skilled in the art. A typical unit dosage form intended for oral administration one or more times per day, such as from one to three times per day, may suitably contain from about 0.05 to about 1000 mg, preferably from about 0.1 to about 500 mg, such as from about 0.5 to about 200 mg of a compound of the invention.

Compounds of the invention comprise compounds that are believed to be well-suited to administration with longer intervals than, for example, once daily, thus, appropriately formulated compounds of the invention may be suitable for, e.g., twice-weekly or once-weekly administration by a suitable route of administration, such as one of the routes disclosed herein.

As described above, compounds of the present invention may be administered or applied in combination with one or more additional therapeutically active compounds or substances, and suitable additional compounds or substances may be selected, for example, from antidiabetic agents, antihyperlipidemic agents, antiobesity agents, antihypertensive agents and agents for the treatment of complications resulting from, or associated with, diabetes.

Suitable antidiabetic agents include insulin, insulin derivatives or analogues, GLP-1 (glucagon like peptide-1) derivatives or analogues [such as those disclosed in WO 98/08871 (Novo Nordisk NS), or other GLP-1 analogues such as exenatide (Byetta, Eli Lilly/Amylin; AVE0010, Sanofi-Aventis), taspoglutide (Roche), albiglutide (Syncria, GlaxoSmithKline), amylin, amylin analogues (e.g. Symlin™/Pramlintide) as well as orally active hypoglycemic agents.

Suitable orally active hypoglycemic agents include: metformin, imidazolines; sulfonylureas; biguanides; meglitinides; oxadiazolidinediones; thiazolidinediones; insulin sensitizers; α-glucosidase inhibitors; agents acting on the ATP-dependent potassium channel of the pancreatic β-cells, e.g. potassium channel openers such as those disclosed in WO 97/26265, WO 99/03861 and WO 00/37474 (Novo Nordisk NS); potassium channel openers such as ormitiglinide; potassium channel blockers such as nateglinide or BTS-67582; glucagon receptor antagonists such as those disclosed in WO 99/01423 and WO 00/39088 (Novo Nordisk A/S and Agouron Pharmaceuticals, Inc.); GLP-1 receptor agonists such as those disclosed in WO 00/42026 (Novo Nordisk A/S and Agouron Pharmaceuticals, Inc); amylin analogues (agonists on the amylin receptor); DPP-IV (dipeptidyl peptidase-IV) inhibitors; PTPase (protein tyrosine phosphatase) inhibitors; glucokinase activators, such as those described in WO 02/08209 to Hoffmann La Roche; inhibitors of hepatic enzymes involved in stimulation of gluconeogenesis and/or glycogenolysis; glucose uptake modulators; GSK-3 (glycogen synthase kinase-3) inhibitors; compounds modifying lipid metabolism, such as antihyperlipidemic agents and antilipidemic agents; compounds lowering food intake; as well as PPAR (peroxisome proliferator-activated receptor) agonists and RXR (retinoid X receptor) agonists such as ALRT-268, LG-1268 or LG-1069.

Other examples of suitable additional therapeutically active substances include insulin or insulin analogues; sulfonylureas, e.g. tolbutamide, chlorpropamide, tolazamide, glibenclamide, glipizide, glimepiride, glicazide or glyburide; biguanides, e.g. metformin; and meglitinides, e.g. repaglinide or senaglinide/nateglinide.

Further examples of suitable additional therapeutically active substances include thiazolidinedione insulin sensitizers, e.g. troglitazone, ciglitazone, pioglitazone, rosiglitazone, isaglitazone, darglitazone, englitazone, CS-011/CI-1037 or T 174, or the compounds disclosed in WO 97/41097 (DRF-2344), WO 97/41119, WO 97/41120, WO 00/41121 and WO 98/45292 (Dr. Reddy's Research Foundation).

Additional examples of suitable additional therapeutically active substances include insulin sensitizers, e.g. GI 262570, YM-440, MCC-555, JTT-501, AR-H039242, KRP-297, GW-409544, CRE-16336, AR-H049020, LY510929, MBX-102, CLX-0940, GW-501516 and the compounds disclosed in WO 99/19313 (NN622/DRF-2725), WO 00/50414, WO 00/63191, WO 00/63192 and WO 00/63193 (Dr. Reddy's Research Foundation), and in WO 00/23425, WO 00/23415, WO 00/23451, WO 00/23445, WO 00/23417, WO 00/23416, WO 00/63153, WO 00/63196, WO 00/63209, WO 00/63190 and WO 00/63189 (Novo Nordisk A/S).

Still further examples of suitable additional therapeutically active substances include: α-glucosidase inhibitors, e.g. voglibose, emiglitate, miglitol or acarbose; glycogen phosphorylase inhibitors, e.g. the compounds described in WO 97/09040 (Novo Nordisk NS); glucokinase activators; agents acting on the ATP-dependent potassium channel of the pancreatic β-cells, e.g. tolbutamide, glibenclamide, glipizide, glicazide, BTS-67582 or repaglinide;

Other suitable additional therapeutically active substances include antihyperlipidemic agents and antilipidemic agents, e.g. cholestyramine, colestipol, clofibrate, gemfibrozil, lovastatin, pravastatin, simvastatin, probucol or dextrothyroxine.

Further agents which are suitable as additional therapeutically active substances include antiobesity agents and appetite-regulating agents. Such substances may be selected from the group consisting of CART (cocaine amphetamine regulated transcript) agonists, NPY (neuropeptide Y receptor 1 and/or 5) antagonists, MC3 (melanocortin receptor 3) agonists, MC3 antagonists, MC4 (melanocortin receptor 4) agonists, orexin receptor antagonists, TNF (tumor necrosis factor) agonists, CRF (corticotropin releasing factor) agonists, CRF BP (corticotropin releasing factor binding protein) antagonists, urocortin agonists, neuromedin U analogues (agonists on the neuromedin U receptor subtypes 1 and 2), β3 adrenergic agonists such as CL-316243, AJ-9677, GW-0604, LY362884, LY377267 or AZ-40140, MC1 (melanocortin receptor 1) agonists, MCH (melanocyte-concentrating hormone) antagonists, CCK (cholecystokinin) agonists, serotonin reuptake inhibitors (e.g. fluoxetine, seroxat or citalopram), serotonin and norepinephrine reuptake inhibitors, 5HT (serotonin) agonists, 5HT6 agonists, 5HT2c agonists such as APD356 (U.S. Pat. No. 6,953,787), bombesin agonists, galanin antagonists, growth hormone, growth factors such as prolactin or placental lactogen, growth hormone releasing compounds, TRH (thyrotropin releasing hormone) agonists, UCP 2 or 3 (uncoupling protein 2 or 3) modulators, chemical uncouplers, leptin agonists, DA (dopamine) agonists (bromocriptin, doprexin), lipase/amylase inhibitors, PPAR modulators, RXR modulators, TR β agonists, adrenergic CNS stimulating agents, AGRP (agouti-related protein) inhibitors, histamine H3 receptor antagonists such as those disclosed in WO 00/42023, WO 00/63208 and WO 00/64884, exendin-4 analogues, GLP-1 analogues, ciliary neurotrophic factor, amylin analogues, peptide YY₃₋₃₆ (PYY3-36) (Batterham et al, Nature 418, 650-654 (2002)), PYY3-36 analogues, NPY Y2 receptor agonists, NPY Y4 receptor agonists and substances acting as combined NPY Y2 and NPY Y4 agonists, FGF21 and analogues thereof, μ-opioid receptor antagonists, oxyntomodulin or analogues thereof.

Further suitable antiobesity agents are bupropion (antidepressant), topiramate (anticonvulsant), ecopipam (dopamine D1/D5 antagonist) and naltrexone (opioid antagonist), and combinations thereof. Combinations of these antiobesity agents would be e.g.: phentermine+topiramate, bupropion sustained release (SR)+naltrexone SR, zonisamide SR and bupropion SR. Among embodiments of suitable antiobesity agents for use in a method of the invention as additional therapeutically active substances in combination with a compound of the invention are leptin and analogues or derivatives of leptin.

Additional embodiments of suitable antiobesity agents are serotonin and norepinephrine reuptake inhibitors, e.g. sibutramine.

Other embodiments of suitable antiobesity agents are lipase inhibitors, e.g. orlistat.

Still further embodiments of suitable antiobesity agents are adrenergic CNS stimulating agents, e.g. dexamphetamine, amphetamine, phentermine, mazindol, phendimetrazine, diethylpropion, fenfluramine or dexfenfluramine.

Other examples of suitable additional therapeutically active compounds include antihypertensive agents. Examples of antihypertensive agents are β-blockers such as alprenolol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin.

The compounds of the present invention have higher glucagon receptor selectivity in relation to previously disclosed peptides in the art. The peptides of the present invention also have prolonged in vivo half-life. The compounds of the present invention can be a soluble glucagon receptor agonist, for example with solubility of at least 0.2 mmol/l, at least 0.5 mmol/l, at least 2 mmol/l, at least 4 mmol/l, at least 8 mmol/l, at least 10 mmol/l, or at least 15 mmol/l.

In the present context, if not stated otherwise, the terms “soluble”, “solubility”, “soluble in aqueous solution”, “aqueous solubility”, “water soluble”, “water-soluble”, “water solubility” and “water-solubility”, refer to the solubility of a compound in water or in an aqueous salt or aqueous buffer solution, for example a 10 mM phosphate solution, or in an aqueous solution containing other compounds, but no organic solvents.

The term “polypeptide” and “peptide” as used herein means a compound composed of at least five constituent amino acids connected by peptide bonds. The constituent amino acids may be from the group of the amino acids encoded by the genetic code and they may be natural amino acids which are not encoded by the genetic code, as well as synthetic amino acids. Natural amino acids which are not encoded by the genetic code are e.g. hydroxyproline, γ-carboxyglutamate, ornithine, phosphoserine, D-alanine and D-glutamine. Synthetic amino acids comprise amino acids manufactured by chemical synthesis, i.e. D-isomers of the amino acids encoded by the genetic code such as D-alanine and D-leucine, Aib (α-aminoisobutyric acid), Abu (α-aminobutyric acid), Tle (tert-butylglycine), β-alanine, 3-aminomethyl benzoic acid, anthranilic acid.

The term “analogue” as used herein referring to a polypeptide means a modified peptide wherein one or more amino acid residues of the peptide have been substituted by other amino acid residues and/or wherein one or more amino acid residues have been deleted from the peptide and/or wherein one or more amino acid residues have been deleted from the peptide and or wherein one or more amino acid residues have been added to the peptide. Such addition or deletion of amino acid residues can take place at the N-terminal of the peptide and/or at the C-terminal of the peptide. A simple system is used to describe analogues. Formulae of peptide analogs and derivatives thereof are drawn using standard single letter or three letter abbreviations for amino acids used according to IUPAC-IUB nomenclature.

The term “derivative” as used herein in relation to a peptide means a chemically modified peptide or an analogue thereof, wherein at least one substituent is not present in the unmodified peptide or an analogue thereof, i.e. a peptide which has been covalently modified. Typical modifications are amides, carbohydrates, alkyl groups, acyl groups, esters and the like.

All amino acids for which the optical isomer is not stated is to be understood to mean the L-isomer.

The term “glucagon peptide” as used herein means glucagon compound, glucagon analogues, glucagon peptide analogue, derivative of glucagon peptide analogue, derivative of glucagon analogue, derivative of glucagon peptide, glucagon peptide derivative, compound according to the present invention, compound of the present invention, compound, amino acid sequence SEQ ID 1, the amino acid sequence of formula I, peptide of formula I, glucagon peptide of formula, a glucagon analogue of SEQ ID 1, a glucagon derivative or a derivative of SEQ ID 1, human glucagon(1-29), glucagon(1-30), glucagon(1-31), glucagon(1-32) as well as analogues, fusion peptides, and derivatives thereof, which maintain glucagon activity.

As regards position numbering in glucagon compounds: for the present purposes any amino acid substitution, deletion, and/or addition is indicated relative to the sequences of native human glucagon (1-29) (SEQ ID 1). Human glucagon amino acids positions 1-29 are herein to be the same as amino acid positions X₁ to X₂₉. The human glucagon (1-29) sequence is His-Ser-Gln-Gly-Thr-Phe-Thr-Ser-Asp-Tyr-Ser-Lys-Tyr-Leu-Asp-Ser-Arg-Arg-Ala-Gln-Asp-Phe-Val-Gln-Trp-Leu-Met-Asn-Thr (SEQ ID 1).

Glucagon(1-30) means human glucagon with an extension of one amino acid in the C-terminal, glucagon(1-31) means human glucagon with an extension of two amino acid in the C-terminal and glucagon(1-32) means human glucagon with an extension of three amino acid in the C-terminal.

The term “negative charged moiety” as used herein, means a negatively chargeable chemical moiety such as, but not limited to an amino acid moiety such as Glu, γGlu, Asp or βAsp, a carboxylic acid, sulphonic acid or a tetrazole moiety.

The term “substituent” as used herein, means a chemical moiety or group replacing a hydrogen.

The term “C₂₋₆ acyl group” as used herein, means a branched or unbranched acyl group with two to six carbon atoms such as:

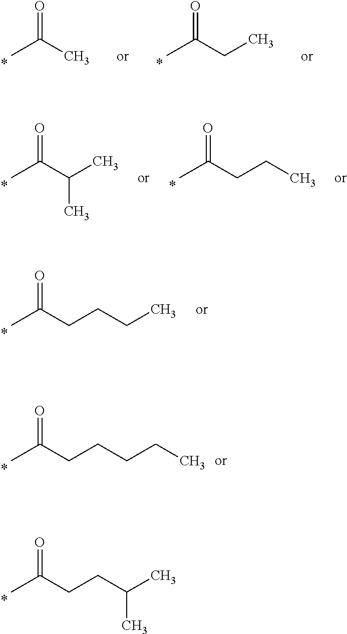

wherein * represents the point of attachment to the neighbouring position.
The term succinoyl as used herein refer to the following moiety:

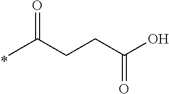

where * represents the point of attachment to the neighbouring position.

The term “lipophilic moiety” as used herein, means an aliphatic or cyclic hydrocarbon moiety with more than 6 and less than 30 carbon atoms, wherein said hydrocarbon moiety may contain additional substituents.

Further embodiments of the present invention relate to:
91. A fast-acting glucagon peptide derivative according to any one of the previous embodiments, with onset of action of the hyperglycemic effect equivalent to that of native glucagon after subcutaneous administration.
92. A fast-acting glucagon peptide derivative according to any one of the previous embodiments, with onset of action of the hyperglycemic effect equivalent to that of native glucagon after subcutaneous or intramuscular administration.
93. A fast-acting glucagon peptide derivative according to any one of the previous embodiments, with improved bioavailability after subcutaneous administration.
94. The derivative of glucagon peptide analogue according to any one of the previous embodiments, with improved bioavailability after subcutaneous or intramuscular administration.
95. A derivative of glucagon peptide analogue according to any of the previous embodiments, wherein said glucagon peptide is a DPPIV protected compound.
96. A derivative of glucagon peptide analogue according to any of the previous embodiments, wherein said glucagon peptide is DPPIV stabilised.
97. A derivative of glucagon peptide analogue according to any of the previous embodiments, wherein said glucagon peptide is an agonist of the glucagon receptor.
98. A derivative of glucagon peptide analogue according to embodiment 97, wherein said glucagon peptide is an agonist of the glucagon receptor, with an EC₅₀<1 nM.
99. A derivative of glucagon peptide analogue according to any of the previous embodiments, wherein said glucagon peptide has more than 70% recovery in the ThT fibrillation assay.
100. A derivative of glucagon peptide analogue according to embodiments 1-98, wherein said glucagon peptide has more than 90% recovery in the ThT fibrillation assay.
101. A derivative of glucagon peptide analogue according to to embodiments 1-98, wherein said glucagon peptide has about 100% recovery in the ThT fibrillation assay.
102. A derivative of glucagon peptide analogue according to any of the previous embodiments, wherein said glucagon peptide has more than 7 hours lag time in the ThT fibrillation assay.
103. A derivative of glucagon peptide analogue according to any of the previous embodiments, wherein said glucagon peptide has more than 20 hours lag time in the ThT fibrillation assay.
104. A derivative of glucagon peptide analogue according to any of the previous embodiments, wherein said glucagon peptide has 45 hours lag time or more in the ThT fibrillation assay.

The term “DPP-IV protected” as used herein referring to a polypeptide means a polypeptide which has been chemically modified in order to render said compound resistant to the plasma peptidase dipeptidyl aminopeptidase-4 (DPP-IV). The DPP-IV enzyme in plasma is known to be involved in the degradation of several peptide hormones, e.g. glucagon, GLP-1, GLP-2, oxyntomodulin etc. Thus, a considerable effort is being made to develop analogues and derivatives of the polypeptides susceptible to DPP-IV mediated hydrolysis in order to reduce the rate of degradation by DPP-IV.

The term “glucagon agonist” as used herein refers to any glucagon peptide which fully or partially activates the human glucagon receptor. In a preferred embodiment, the “glucagon agonist” is any glucagon peptide that binds to a glucagon receptor, preferably with an affinity constant (KD) or a potency (EC₅₀) of below 1 μM, e.g., below 100 nM or below 1 nM, as measured by methods known in the art and exhibits insulinotropic activity, where insulinotropic activity may be measured in vivo or in vitro assays known to those of ordinary skill in the art. For example, the glucagon agonist may be administered to an animal and the insulin concentration measured over time.

In the present context, the term “agonist” is intended to indicate a substance (ligand) that activates the receptor type in question.

In the present context, the term “pharmaceutically acceptable salt” is intended to indicate a salt which is not harmful to the patient. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric and nitric acids, and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene-salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. (1977) 66, 2. Examples of relevant metal salts include lithium, sodium, potassium and magnesium salts, and the like. Examples of alkylated ammonium salts include methylammonium, dimethylammonium, trimethylammonium, ethylammonium, hydroxyethylammonium, diethylammonium, butylammonium and tetramethylammonium salts, and the like.

As use herein, the term “therapeutically effective amount” of a compound refers to an amount sufficient to cure, alleviate or partially arrest the clinical manifestations of a given disease and/or its complications. An amount adequate to accomplish this is defined as a “therapeutically effective amount”. Effective amounts for each purpose will depend on the severity of the disease or injury, as well as on the weight and general state of the subject. It will be understood that determination of an appropriate dosage may be achieved using routine experimentation, by constructing a matrix of values and testing different points in the matrix, all of which is within the level of ordinary skill of a trained physician or veterinarian.

The terms “treatment”, “treating” and other variants thereof as used herein refer to the management and care of a patient for the purpose of combating a condition, such as a disease or a disorder. The terms are intended to include the full spectrum of treatments for a given condition from which the patient is suffering, such as administration of the active compound(s) in question to alleviate symptoms or complications thereof, to delay the progression of the disease, disorder or condition, to cure or eliminate the disease, disorder or condition, and/or to prevent the condition, in that prevention is to be understood as the management and care of a patient for the purpose of combating the disease, condition, or disorder, and includes the administration of the active compound(s) in question to prevent the onset of symptoms or complications. The patient to be treated is preferably a mammal, in particular a human being, but treatment of other animals, such as dogs, cats, cows, horses, sheep, goats or pigs, is within the scope of the invention.

As used herein, the term “solvate” refers to a complex of defined stoichiometry formed between a solute (in casu, a compound according to the present invention) and a solvent. Solvents may include, by way of example, water, ethanol, or acetic acid.

Other embodiments of the present relates to pharmaceutical compositions:
105. A pharmaceutical composition comprising a derivative of glucagon peptide analogue according to any one of embodiments 1-104.
106. The pharmaceutical composition according to embodiment 105, further comprising one or more additional therapeutically active compounds or substances.
107. The pharmaceutical composition according to any one of embodiments 105-106, in unit dosage form comprising from about 0.05 mg to about 1000 mg, such as from about 0.1 mg to about 500 mg, from about 0.011 mg to about 5 mg, from about 0.5 mg to about 2 mg, from about 0.5 mg to about 5 mg, e.g. from about 0.5 mg to about 200 mg, of a compound according to any of embodiments 1-104.
108. The pharmaceutical composition according to any one of embodiment 107, in unit dosage form comprising from about 0.01 mg to about 4 mg, of a compound according to any of embodiments 1-104.
109. The pharmaceutical composition according to any one of embodiment 107, in unit dosage form comprising from about 0.05 mg to about 3 mg, of a compound according to any of embodiments 1-104.
110. The pharmaceutical composition according to any one of embodiment 107, in unit dosage form comprising from about 0.05 mg to about 2 mg, of a compound according to any of embodiments 1-104.
111. The pharmaceutical composition according to any one of embodiment 107, in unit dosage form comprising from about 0.1 mg to about 1 mg, of a compound according to any of embodiments 1-104.
112. The pharmaceutical composition according to any one of embodiment 107, in unit dosage form comprising from about 0.1 mg to about 2 mg, of a compound according to any of embodiments 1-104.
113. The pharmaceutical composition according to any one of embodiments 105-112, which is suited for parenteral administration.
114. A glucagon peptide according to any of any one of embodiments 1-104, for use in therapy.
   Further embodiments of the present invention relate to the following:
115. The derivative of glucagon peptide analogue according to any of embodiments 1-104, optionally in combination with one or more additional therapeutically active compounds, for use in treatment or prevention of hyperglycemia, type 2 diabetes, impaired glucose tolerance, type 1 diabetes and obesity.
116. The derivative of glucagon peptide analogue according to any of embodiments 1-104, optionally in combination with one or more additional therapeutically active compounds, for use in delaying or preventing disease progression in type 2 diabetes.
117. The derivative of glucagon peptide analogue according to any of embodiments 1-104, optionally in combination with one or more additional therapeutically active compounds, for use treating obesity or preventing overweight.
118. The derivative of glucagon peptide analogue according to any of embodiments 1-104, optionally in combination with one or more additional therapeutically active compounds, for use in for decreasing food intake.
119. The derivative of glucagon peptide analogue according to any of embodiments 1-104, optionally in combination with one or more additional therapeutically active compounds, for use in increasing energy expenditure.
120. The derivative of glucagon peptide analogue according to any of embodiments 1-104, optionally in combination with one or more additional therapeutically active compounds, for use in reducing body weight.
121. The derivative of glucagon peptide analogue according to any of embodiments 1-104, optionally in combination with one or more additional therapeutically active compounds, for use in delaying the progression from impaired glucose tolerance (IGT) to type 2 diabetes.
122. The derivative of glucagon peptide analogue according to any of embodiments 1-104, optionally in combination with one or more additional therapeutically active compounds, for use in delaying the progression from type 2 diabetes to insulin-requiring diabetes.
123. The derivative of glucagon peptide analogue according to any of embodiments 1-104, optionally in combination with one or more additional therapeutically active compounds, for use regulating appetite.
124. The derivative of glucagon peptide analogue according to any of embodiments 1-104, optionally in combination with one or more additional therapeutically active compounds, for use inducing satiety.
125. The derivative of glucagon peptide analogue according to any of embodiments 1-104, optionally in combination with one or more additional therapeutically active compounds, for use in preventing weight regain after successful weight loss.
126. The derivative of glucagon peptide analogue according to any of embodiments 1-104, optionally in combination with one or more additional therapeutically active compounds, for use in treating a disease or state related to overweight or obesity.
127. The derivative of glucagon peptide analogue according to any of embodiments 1-104, optionally in combination with one or more additional therapeutically active compounds, for use in treating bulimia.
128. The derivative of glucagon peptide analogue according to any of embodiments 1-104, optionally in combination with one or more additional therapeutically active compounds, for use in treating binge-eating.
129. The derivative of glucagon peptide analogue according to any of embodiments 1-104, optionally in combination with one or more additional therapeutically active compounds, for use in treating atherosclerosis.
130. The derivative of glucagon peptide analogue according to any of embodiments 1-104, optionally in combination with one or more additional therapeutically active compounds, for use in treating hypertension.
131. The derivative of glucagon peptide analogue according to any of embodiments 1-104, optionally in combination with one or more additional therapeutically active compounds, for use in treating type 2 diabetes.
132. The derivative of glucagon peptide analogue according to any of embodiments 1-104, optionally in combination with one or more additional therapeutically active compounds, for use in treating impaired glucose tolerance.
133. The derivative of glucagon peptide analogue according to any of embodiments 1-104, optionally in combination with one or more additional therapeutically active compounds, for use in treating dyslipidemia.
134. The derivative of glucagon peptide analogue according to any of embodiments 1-104, optionally in combination with one or more additional therapeutically active compounds, for use in treating coronary heart disease.
135. The derivative of glucagon peptide analogue according to any of embodiments 1-104, optionally in combination with one or more additional therapeutically active compounds, for use in treating hepatic steatosis.
136. The derivative of glucagon peptide analogue according to any of embodiments 1-104, optionally in combination with one or more additional therapeutically active compounds, for use in treating hepatic steatosis.
137. The derivative of glucagon peptide analogue according to any of embodiments 1-104, optionally in combination with one or more additional therapeutically active compounds, for use in treating beta-blocker poisoning.
138. The derivative of glucagon peptide analogue according to any of embodiments 1-104, optionally in combination with one or more additional therapeutically active compounds, for use in inhibition of the motility of the gastrointestinal tract, useful in connection with investigations of the gastrointestinal tract using techniques such as x-ray, CT- and NMR-scanning.
139. The derivative of glucagon peptide analogue according to any of embodiments 1-104, optionally in combination with one or more additional therapeutically active compounds, for use in treatment or prevention of hypoglycaemia.
140. The derivative of glucagon peptide analogue according to any of embodiments 1-104, optionally in combination with one or more additional therapeutically active compounds, for use in treatment or prevention of insulin induced hypoglycaemia.
141. The derivative of glucagon peptide analogue according to any of embodiments 1-104, optionally in combination with one or more additional therapeutically active compounds, for use in treatment or prevention of reactive hypoglycaemia.
142. The derivative of glucagon peptide analogue according to any of embodiments 1-104, optionally in combination with one or more additional therapeutically active compounds, for use in treatment or prevention of diabetic hypoglycaemia.
143. The derivative of glucagon peptide analogue according to any of embodiments 1-104, optionally in combination with one or more additional therapeutically active compounds, for use in treatment or prevention of non-diabetic hypoglycaemia.
144. The derivative of glucagon peptide analogue according to any of embodiments 1-104, optionally in combination with one or more additional therapeutically active compounds, for use in treatment or prevention of fasting hypoglycaemia.
145. The derivative of glucagon peptide analogue according to any of embodiments 1-104, optionally in combination with one or more additional therapeutically active compounds, for use in treatment or prevention of drug-induced hypoglycaemia.
146. The derivative of glucagon peptide analogue according to any of embodiments 1-104, optionally in combination with one or more additional therapeutically active compounds, for use in treatment or prevention of gastric by-pass induced hypoglycaemia.
147. The derivative of glucagon peptide analogue according to any of embodiments 1-104, optionally in combination with one or more additional therapeutically active compounds, for use in treatment or prevention of hypoglycemia in pregnancy.
148. The derivative of glucagon peptide analogue according to any of embodiments 1-104, optionally in combination with one or more additional therapeutically active compounds, for use in treatment or prevention of alcohol-induced hypoglycaemia.
149. The derivative of glucagon peptide analogue according to any of embodiments 1-104, optionally in combination with one or more additional therapeutically active compounds, for use in treatment or prevention of insulinoma.
150. The derivative of glucagon peptide analogue according to any of embodiments 1-104, optionally in combination with one or more additional therapeutically active compounds, for use in treatment or prevention of Von Girkes disease.
   Further embodiments of the present invention relate to the following methods:
151. A method for treating or preventing hyperglycemia, type 2 diabetes, impaired glucose tolerance, type 1 diabetes and obesity, comprising administering to a patient in need thereof, an effective amount of a derivative of glucagon peptide analogue according to any of embodiments 1-104, optionally in combination with one or more additional therapeutically active compounds.
152. A method for delaying or preventing disease progression in type 2 diabetes, comprising administering to a patient in need thereof, an effective amount of a derivative of glucagon peptide analogue according to any of embodiments 1-104, optionally in combination with one or more additional therapeutically active compounds.
153. A method for treating obesity or preventing overweight, comprising administering to a patient in need thereof, an effective amount of a derivative of glucagon peptide analogue according to any of embodiments 1-104, optionally in combination with one or more additional therapeutically active compounds.
154. A method for decreasing food intake, comprising administering to a patient in need thereof, an effective amount of a derivative of glucagon peptide analogue according to any of embodiments 1-104, optionally in combination with one or more additional therapeutically active compounds.
155. A method for use in increasing energy expenditure, comprising administering to a patient in need thereof, an effective amount of a derivative of glucagon peptide analogue according to any of embodiments 1-104, optionally in combination with one or more additional therapeutically active compounds.
156. A method for use in reducing body weight, comprising administering to a patient in need thereof, an effective amount of a derivative of glucagon peptide analogue according to any of embodiments 1-104, optionally in combination with one or more additional therapeutically active compounds.
157. A method for use in delaying the progression from impaired glucose tolerance (IGT) to type 2 diabetes, comprising administering to a patient in need thereof, an effective amount of a derivative of glucagon peptide analogue according to any of embodiments 1-104, optionally in combination with one or more additional therapeutically active compounds.
158. A method for use in delaying the progression from type 2 diabetes to insulin-requiring diabetes, comprising administering to a patient in need thereof, an effective amount of a derivative of glucagon peptide analogue according to any of embodiments 1-104, optionally in combination with one or more additional therapeutically active compounds.
159. A method for use in regulating appetite, comprising administering to a patient in need thereof, an effective amount of a derivative of glucagon peptide analogue according to any of embodiments 1-104, optionally in combination with one or more additional therapeutically active compounds.
160. A method for use in inducing satiety, comprising administering to a patient in need thereof, an effective amount of a derivative of glucagon peptide analogue according to any of embodiments 1-104, optionally in combination with one or more additional therapeutically active compounds.
161. A method for use in preventing weight regain after successful weight loss, comprising administering to a patient in need thereof, an effective amount of a derivative of glucagon peptide analogue according to any of embodiments 1-104, optionally in combination with one or more additional therapeutically active compounds.
162. A method for use in treating a disease or state related to overweight or obesity, comprising administering to a patient in need thereof, an effective amount of a derivative of glucagon peptide analogue according to any of embodiments 1-104, optionally in combination with one or more additional therapeutically active compounds.
163. A method for use in treating bulimia, comprising administering to a patient in need thereof, an effective amount of a derivative of glucagon peptide analogue according to any of embodiments 1-104, optionally in combination with one or more additional therapeutically active compounds.
164. A method for use in treating binge-eating, comprising administering to a patient in need thereof, an effective amount of a derivative of glucagon peptide analogue according to any of embodiments 1-104, optionally in combination with one or more additional therapeutically active compounds.
165. A method for use in treating atherosclerosis, comprising administering to a patient in need thereof, an effective amount of a derivative of glucagon peptide analogue according to any of embodiments 1-104, optionally in combination with one or more additional therapeutically active compounds.
166. A method for use in treating hypertension, comprising administering to a patient in need thereof, an effective amount of a derivative of glucagon peptide analogue according to any of embodiments 1-104, optionally in combination with one or more additional therapeutically active compounds.
167. A method for use in treating type 2 diabetes, comprising administering to a patient in need thereof, an effective amount of a derivative of glucagon peptide analogue according to any of embodiments 1-104, optionally in combination with one or more additional therapeutically active compounds.
168. A method for use in treating impaired glucose tolerance, comprising administering to a patient in need thereof, an effective amount of a derivative of glucagon peptide analogue according to any of embodiments 1-104, optionally in combination with one or more additional therapeutically active compounds.
169. A method for use in treating dyslipidemia, comprising administering to a patient in need thereof, an effective amount of a derivative of glucagon peptide analogue according to any of embodiments 1-104, optionally in combination with one or more additional therapeutically active compounds.
170. A method for use in treating coronary heart disease, comprising administering to a patient in need thereof, an effective amount of a derivative of glucagon peptide analogue according to any of embodiments 1-104, optionally in combination with one or more additional therapeutically active compounds.
171. A method for use in treating hepatic steatosis, comprising administering to a patient in need thereof, an effective amount of a derivative of glucagon peptide analogue according to any of embodiments 1-104, optionally in combination with one or more additional therapeutically active compounds.
172. A method for use in treating beta-blocker poisoning, comprising administering to a patient in need thereof, an effective amount of a derivative of glucagon peptide analogue according to any of embodiments 1-104, optionally in combination with one or more additional therapeutically active compounds.
173. A method for use in inhibition of the motility of the gastrointestinal tract, useful in connection with investigations of the gastrointestinal tract using techniques such as x-ray, CT- and NMR-scanning, comprising administering to a patient in need thereof, an effective amount of a derivative of glucagon peptide analogue according to any of embodiments 1-104, optionally in combination with one or more additional therapeutically active compounds.
174. A method for use in treatment or prevention of hypoglycaemia, comprising administering to a patient in need thereof, an effective amount of a derivative of glucagon peptide analogue according to any of embodiments 1-104, optionally in combination with one or more additional therapeutically active compounds.
175. A method for use in treatment or prevention of insulin induced hypoglycaemia, comprising administering to a patient in need thereof, an effective amount of a derivative of glucagon peptide analogue according to any of embodiments 1-104, optionally in combination with one or more additional therapeutically active compounds.
176. A method for use in treatment or prevention of reactive hypoglycaemia, comprising administering to a patient in need thereof, an effective amount of a derivative of glucagon peptide analogue according to any of embodiments 1-104, optionally in combination with one or more additional therapeutically active compounds.
177. A method for use in treatment or prevention of diabetic hypoglycaemia, comprising administering to a patient in need thereof, an effective amount of a derivative of glucagon peptide analogue according to any of embodiments 1-104, optionally in combination with one or more additional therapeutically active compounds.
178. A method for use in treatment or prevention of non-diabetic hypoglycaemia, comprising administering to a patient in need thereof, an effective amount of a derivative of glucagon peptide analogue according to any of embodiments 1-104, optionally in combination with one or more additional therapeutically active compounds.
179. A method for use in treatment or prevention of fasting hypoglycaemia, comprising administering to a patient in need thereof, an effective amount of a derivative of glucagon peptide analogue according to any of embodiments 1-104, optionally in combination with one or more additional therapeutically active compounds.
180. A method for use in treatment or prevention of drug-induced hypoglycaemia, comprising administering to a patient in need thereof, an effective amount of a derivative of glucagon peptide analogue according to any of embodiments 1-104, optionally in combination with one or more additional therapeutically active compounds.
181. A method for use in treatment or prevention of gastric by-pass induced hypoglycaemia, comprising administering to a patient in need thereof, an effective amount of a derivative of glucagon peptide analogue according to any of embodiments 1-104, optionally in combination with one or more additional therapeutically active compounds.
182. A method for use in treatment or prevention of hypoglycemia in pregnancy, comprising administering to a patient in need thereof, an effective amount of a derivative of glucagon peptide analogue according to any of embodiments 1-104, optionally in combination with one or more additional therapeutically active compounds.
183. A method for use in treatment or prevention of alcohol-induced hypoglycaemia, comprising administering to a patient in need thereof, an effective amount of a derivative of glucagon peptide analogue according to any of embodiments 1-104, optionally in combination with one or more additional therapeutically active compounds.
184. A method for use in treatment or prevention of insulinoma, comprising administering to a patient in need thereof, an effective amount of a derivative of glucagon peptide analogue according to any of embodiments 1-104, optionally in combination with one or more additional therapeutically active compounds.
185. A method for use in treatment or prevention of Von Girkes disease, comprising administering to a patient in need thereof, an effective amount of a derivative of glucagon peptide analogue according to any of embodiments 1-104, optionally in combination with one or more additional therapeutically active compounds.
   Further embodiments of the present invention relate to the following uses:
186. Use of a derivative of glucagon peptide analogue according to any one of the embodiments 1-104, for the preparation of a medicament.
187. Use of a derivative of glucagon peptide analogue according to any one of embodiments 1-104, for the preparation of a medicament for the treatment or prevention of hyperglycemia, type 2 diabetes, impaired glucose tolerance, type 1 diabetes and obesity.
188. Use of a derivative of glucagon peptide analogue according to any one of the embodiments 1-104, for the preparation of a medicament for delaying or preventing disease progression in type 2 diabetes, treating obesity or preventing overweight, for decreasing food intake, increase energy expenditure, reducing body weight, delaying the progression from impaired glucose tolerance (IGT) to type 2 diabetes; delaying the progression from type 2 diabetes to insulin-requiring diabetes; regulating appetite; inducing satiety; preventing weight regain after successful weight loss; treating a disease or state related to overweight or obesity; treating bulimia; treating binge-eating; treating atherosclerosis, hypertension, type 2 diabetes, IGT, dyslipidemia, coronary heart disease, hepatic steatosis, treatment of beta-blocker poisoning, use for inhibition of the motility of the gastrointestinal tract, useful in connection with investigations of the gastrointestinal tract using techniques such as x-ray, CT- and NMR-scanning.
189. Use of a derivative of glucagon peptide analogue according to any one of the embodiments 1-104, for the preparation of a medicament for use in inhibition of the motility of the gastrointestinal tract, useful in connection with investigations of the gastrointestinal tract using techniques such as x-ray, CT- and NMR-scanning.
190. Use of a derivative of glucagon peptide analogue according to any one of the embodiments 1-104, for the preparation of a medicament for treatment or prevention of hypoglycemia, insulin induced hypoglycemia, reactive hypoglycemia, diabetic hypoglycemia, non-diabetic hypoglycemia, fasting hypoglycemia, drug-induced hypoglycemia, gastric by-pass induced hypoglycemia, hypoglycemia in pregnancy, alcohol induced hypoglycemia, insulinoma and Von Girkes disease.

In one embodiment the glucagon preparations of the present invention can be used in ready to use pen devices for glucagon administration.

In one embodiment the glucagon preparations of the present invention can be used in pumps for glucagon administration.

The glucagon preparations of the present invention can be used in the treatment of diabetes or hypoglycaemia, by parenteral administration.

It is recommended that the dosage of the glucagon preparations of this invention which is to be administered to the patient be selected by a physician.

Parenteral administration may be performed by subcutaneous, intramuscular, intraperitoneal or intravenous injection by means of a syringe, optionally a pen-like syringe. Alternatively, parenteral administration can be performed by means of an infusion pump. As a further option, the glucagon preparations containing the glucagon compound of the invention can also be adapted to transdermal administration, e.g. by needle-free injection or from a patch, optionally an iontophoretic patch, or transmucosal, e.g. buccal, administration.

Glucagon preparations according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

In certain embodiments of the uses and methods of the present invention, the glucagon peptide of the present invention may be administered or applied in combination with more than one of the above-mentioned, suitable additional therapeutically active compounds or substances, e.g. in combination with: metformin and a sulfonylurea such as glyburide; a sulfonylurea and acarbose; nateglinide and metformin; acarbose and metformin; a sulfonylurea, metformin and troglitazone; insulin and a sulfonylurea; insulin and metformin; insulin, metformin and a sulfonylurea; insulin and troglitazone; insulin and lovastatin; etc.

In the case, in particular, of administration of a glucagon peptide of the invention, optionally in combination with one or more additional therapeutically active compounds or substances as disclosed above, for a purpose related to treatment or prevention of obesity or overweight, i.e. related to reduction or prevention of excess adiposity, it may be of relevance to employ such administration in combination with surgical intervention for the purpose of achieving weight loss or preventing weight gain, e.g. in combination with bariatric surgical intervention. Examples of frequently used bariatric surgical techniques include, but are not limited to, the following: vertical banded gastroplasty (also known as “stomach stapling”), wherein a part of the stomach is stapled to create a smaller pre-stomach pouch which serves as a new stomach; gastric banding, e.g. using an adjustable gastric band system (such as the Swedish Adjustable Gastric Band (SAGB), the LAP-BAND™ or the MIDband™), wherein a small pre-stomach pouch which is to serve as a new stomach is created using an elastomeric (e.g. silicone) band which can be adjusted in size by the patient; and gastric bypass surgery, e.g. “Roux-en-Y” bypass wherein a small stomach pouch is created using a stapler device and is connected to the distal small intestine, the upper part of the small intestine being reattached in a Y-shaped configuration.

The administration of a glucagon peptide of the invention (optionally in combination with one or more additional therapeutically active compounds or substances as disclosed above) may take place for a period prior to carrying out the bariatric surgical intervention in question and/or for a period of time subsequent thereto. In many cases it may be preferable to begin administration of a compound of the invention after bariatric surgical intervention has taken place.

The term “obesity” implies an excess of adipose tissue. When energy intake exceeds energy expenditure, the excess calories are stored in adipose tissue, and if this net positive balance is prolonged, obesity results, i.e. there are two components to weight balance, and an abnormality on either side (intake or expenditure) can lead to obesity. In this context, obesity is best viewed as any degree of excess adipose tissue that imparts a health risk. The distinction between normal and obese individuals can only be approximated, but the health risk imparted by obesity is probably a continuum with increasing adipose tissue. However, in the context of the present invention, individuals with a body mass index (BMI=body weight in kilograms divided by the square of the height in meters) above 25 are to be regarded as obese.

In one embodiment, the present invention relates to a glucagon peptide derivative of formula [I]:

His-X₂-X₃-Gly-Thr-Phe-Thr-Ser-Asp-X₁₀-Ser-X₁₂-Tyr-Leu-X₁₅-X₁₆-Arg-X₁₈-Ala-X₂₀-X₂₁-Phe-Val-X₂₄-Trp-Leu-X₂₇-X₂₈-X₂₉-X₃₀ [I]

comprising a substituent comprising between three and ten negatively charged moieties, attached to the side chain of an amino acid of said glucagon peptide or a pharmaceutically acceptable salt, amide or carboxylic acid thereof, with the proviso that said substituent does not comprise a lipophilic moiety.

In another embodiment, the present invention relates to a glucagon peptide derivative of formula [I]:

His-X₂-X₃-Gly-Thr-Phe-Thr-Ser-Asp-X₁₀-Ser-X₁₂-Tyr-Leu-X₁₅-X₁₆-Arg-X₁₈-Ala-X₂₀-X₂₁-Phe-Val-X₂₄-Trp-Leu-X₂₇-X₂₈-X₂₉-X₃₀ [I]

comprising a substituent comprising between three and ten negatively charged moieties, attached to the side chain of an amino acid of said glucagon peptide or a pharmaceutically acceptable salt, amide or carboxylic acid thereof, with the proviso that said substituent does not comprise a —(CH2)ₙ— moiety, wherein n≧6) moiety.

In another embodiment, the present invention relates to a glucagon peptide derivative of formula [I]:

His-X₂-X₃-Gly-Thr-Phe-Thr-Ser-Asp-X₁₀-Ser-X₁₂-Tyr-Leu-X₁₅-X₁₆-Arg-X₁₈-Ala-X₂₀-X₂₁-Phe-Val-X₂₄-Trp-Leu-X₂₇-X₂₈-X₂₉-X₃₀[I]

comprising a substituent comprising between three and ten negatively charged moieties, attached to the side chain of an amino acid of said glucagon peptide or a pharmaceutically acceptable salt, amide or carboxylic acid thereof, with the proviso that said substituent does not comprise a —(CH₂)₆— moiety.

In another embodiment, the present invention relates to a glucagon peptide derivative of formula [I]:

His-X₂-X₃-Gly-Thr-Phe-Thr-Ser-Asp-X₁₀-Ser-X₁₂-Tyr-Leu-X₁₅-X₁₆-Arg-X₁₈-Ala-X₂₀-X₂₁-Phe-Val-X₂₄-Trp-Leu-X₂₇-X₂₈-X₂₉-X₃₀ [I]

comprising a substituent comprising between three and ten negatively charged moieties, attached to the side chain of an amino acid of said glucagon peptide or a pharmaceutically acceptable salt, amide or carboxylic acid thereof, with the proviso that said substituent does not comprise a moiety selected from the group consisting of:

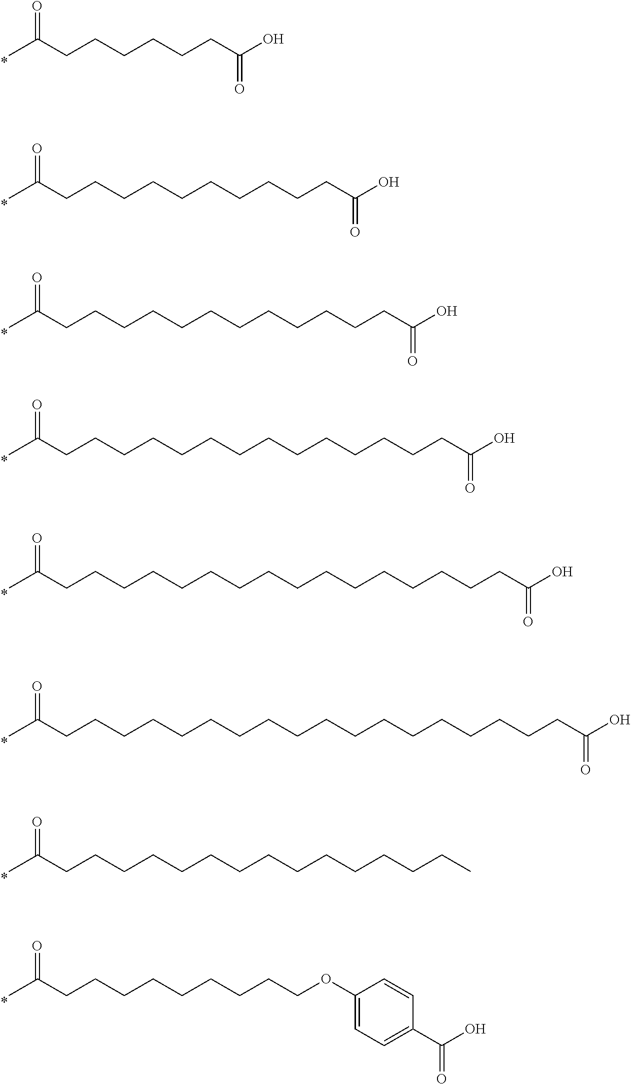

Where * represents the point of attachment to the neighbouring position.

In another embodiment, the present invention relates to a glucagon peptide derivative of formula [I]:

His-X₂-X₃-Gly-Thr-Phe-Thr-Ser-Asp-X₁₀-Ser-X₁₂-Tyr-Leu-X₁₅-X₁₆-Arg-X₁₈-Ala-X₂₀-X₂₁-Phe-Val-X₂₄-Trp-Leu-X₂₇-X₂₈-X₂₉-X₃₀ [I]

comprising a substituent comprising between three and ten negatively charged moieties, attached to the side chain of an amino acid of said glucagon peptide or a pharmaceutically acceptable salt, amide or carboxylic acid thereof, with the proviso that said substituent does not comprise a moiety of formula II:

Z₁—Z₂—Z₃—Z₄ [II]

wherein,
Z₁ represents a structure according to one of the formulas IIa, IIb or IIc;

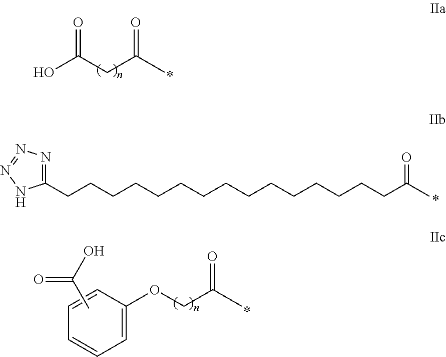

wherein n in formula IIa is 6-20,
m in formula IIc is 5-11
the COOH group in formula IIc can be attached to position 2, 3 or 4 on the phenyl ring,
the symbol * in formula IIa, IIb and IIc represents the attachment point to the nitrogen in Z₂;
if Z₂ is absent, Z₁ is attached to the nitrogen on Z₃ at symbol * and if Z₂ and Z₃ are absent Z₁ is attached to the nitrogen on Z₄ at symbol *
Z₂ is absent or represents a structure according to one of the formulas IId, IIe, IIf, IIg, IIh, IIj or IIk;

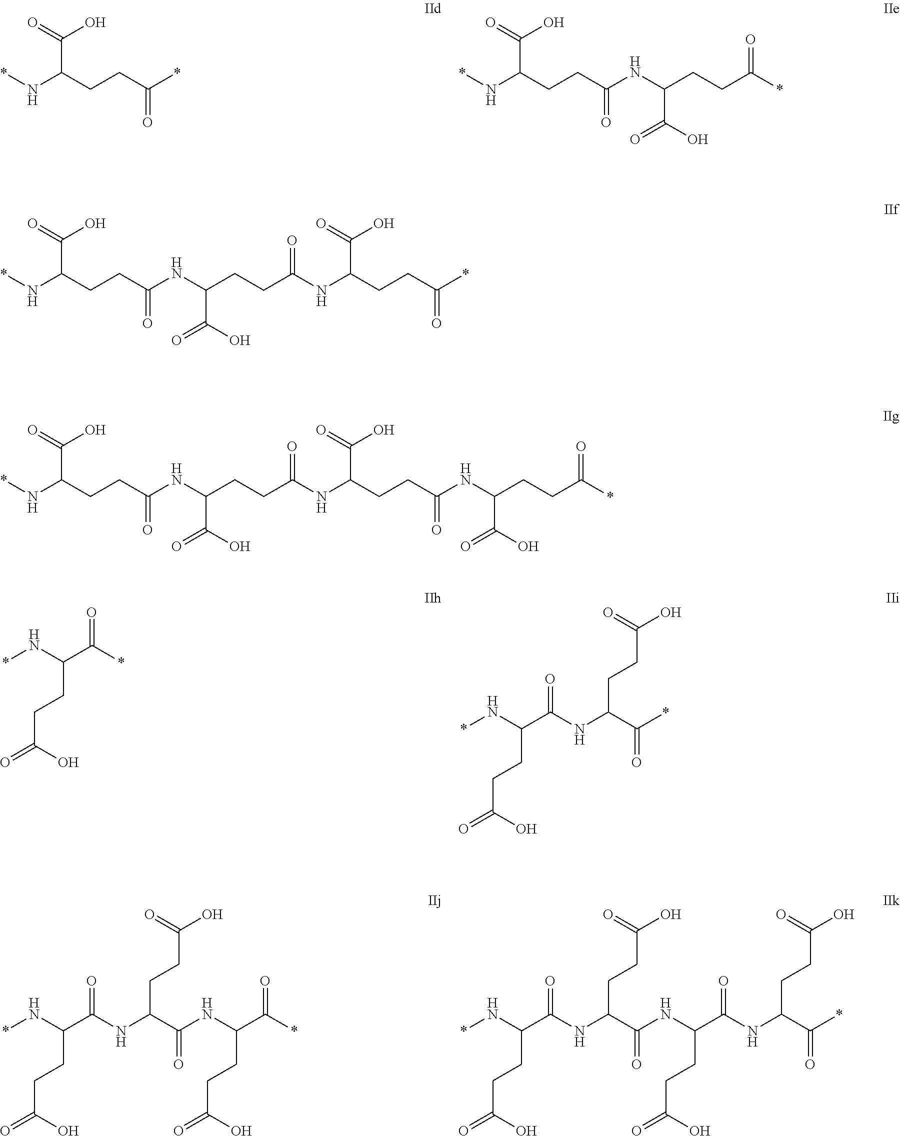

wherein each amino acid moiety independently has the stereochemistry L or D;
wherein Z₂ is connected via the carbon atom denoted * to the nitrogen of Z₃ denoted *;
if Z₃ is absent, Z₂ is connected via the carbon atom denoted * to the nitrogen of Z₄ denoted * and if Z₃ and Z₄ are absent Z₂, is connected via the carbon denoted * to the epsilon nitrogen of a lysine or the delta nitrogen of an ornithine of the glucagon peptide.
Z₃ is absent or represents a structure according to one of the formulas IIm, IIn, IIo or IIp;

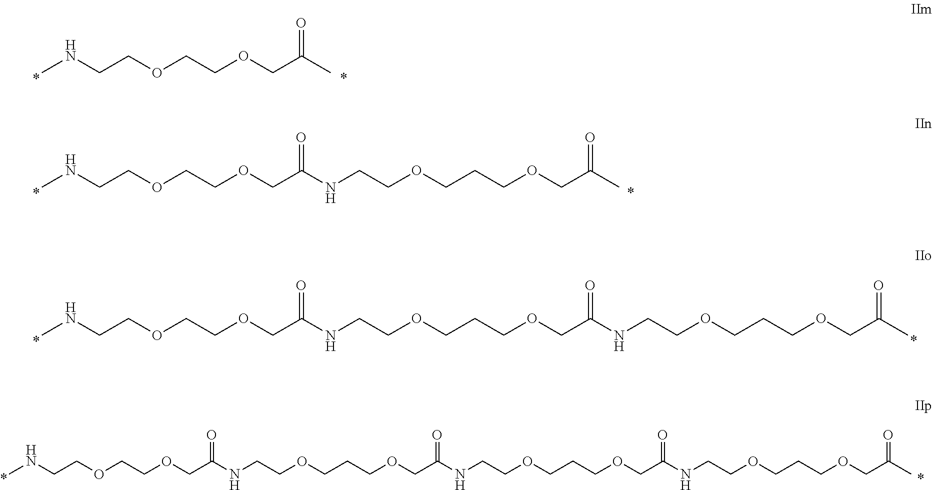

Z₃ is connected vi the carbon of Z₃ with symbol* to the nitrogen of Z₄ with symbol*, if Z₄ is absent Z₃ is connected via the carbon with symbol* to the epsilon nitrogen of a lysine or the delta nitrogen of an ornithine of the glucagon peptide
Z₄ is absent or represents a structure according to one of the formulas IId, IIe, IIf, IIg, IIh, IIj or IIk; wherein each amino acid moiety is independently either L or D, wherein Z₄ is connected via the carbon with symbol* to the epsilon nitrogen of a lysine or the delta nitrogen of an ornithine of the glucagon peptide.

In another embodiment, the present invention relates to a glucagon peptide derivative of formula [I]:

His-X₂-X₃-Gly-Thr-Phe-Thr-Ser-Asp-X₁₀-Ser-X₁₂-Tyr-Leu-X₁₅-X₁₆-Arg-X₁₈-Ala-X₂₀-X₂₁-Phe-Val-X₂₄-Trp-Leu-X₂₇-X₂₈-X₂₉-X₃₀ [I]

comprising a substituent comprising between three and ten negatively charged moieties, attached to the side chain of an amino acid of said glucagon peptide or a pharmaceutically acceptable salt, amide or carboxylic acid thereof, with the proviso that said substituent does not comprise a compound selected from the list consisting of:
Glucagon (1-29)Lys(N-epsilon-((S)-4-carboxy-4-((S)-4-Carboxy-4-((S)-4-carboxy-4-((S)-4-carboxy-4-(19-carboxynonadecanoylamino)-butyrylamino)-butyrylamino)-butyrylamino)-butyryl))-amide;
N-epsilon24-([(4S)-5-hydroxy-4-[[(4S)-5-hydroxy-4-[[2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl]amino]-5-oxopentanoyl]amino]-5-oxopentanoyl])[Lys24,Leu27];
N-epsilon28-([(4S)-5-hydroxy-4-[[(4S)-5-hydroxy-4-[[2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl]amino]-5-oxopentanoyl]amino]-5-oxopentanoyl])[Leu27,Lys28] Glucagon;
N-epsilon29-([(4S)-5-hydroxy-4-[[(4S)-5-hydroxy-4-[[2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl]amino]-5-oxopentanoyl]amino]-5-oxopentanoyl])[Leu27,Lys29];
N-epsilon30-([(4S)-5-hydroxy-4-[[(4S)-5-hydroxy-4-[[2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl]amino]-5-oxopentanoyl]amino]-5-oxopentanoyl])[Leu27,Lys30];
N{Epsilon-28}-[(4S)-4-carboxy-4-[[2-[2-[2-[[2-[2-[2-[[(4S)-4-carboxy-4-[[(4S)-4-carboxy-4-(17-carboxyheptadecanoylamino)butanoyl]amino]butanoyl]amino]ethoxy]ethoxy]acetyl]amino]ethoxy]ethoxy]acetyl]amino]butanoyl]-[Leu27,Lys28]-Glucagon;
N{Epsilon-28}-[2-[2-[2-[[2-[2-[2-[[(4S)-4-carboxy-4-[[(4S)-4-carboxy-4-[[(4S)-4-carboxy-4-(17-carboxyheptadecanoylamino)butanoyl]amino]butanoyl]amino]butanoyl]amino]ethoxy]ethoxy]acetyl]amino]ethoxy]ethoxy]acetyl]-[Leu27,Lys28]-Glucagon;
N{Epsilon-24}-[(4S)-4-carboxy-4-[[2-[2-[2-[[2-[2-[2-[[(4S)-4-carboxy-4-[[(4S)-4-carboxy-4-(17-carboxyheptadecanoylamino)butanoyl]amino]butanoyl]amino]ethoxy]ethoxy]acetyl]amino]ethoxy]ethoxy]acetyl]amino]butanoyl]-[Lys24,Leu27,Ser28]-Glucagon;
N{Epsilon-24}-[2-[2-[2-[[2-[2-[2-[[(4S)-4-carboxy-4-[[(4S)-4-carboxy-4-[[(4S)-4-carboxy-4-(17-carboxyheptadecanoylamino)butanoyl]amino]butanoyl]amino]butanoyl]amino]ethoxy]ethoxy]acetyl]amino]ethoxy]ethoxy]acetyl]-[Lys24,Leu27,Ser28]-Glucagon;
N{Epsilon-16}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys16,Leu27]-Glucagon;
N{Epsilon-24}-[(4S)-4-carboxy-4-[[(4S)-4-carboxy-4-[[(4S)-4-carboxy-4-(17-carboxyheptadecanoylamino)butanoyl]amino]butanoyl]amino]butanoyl]-[Lys24,Leu27,Ser28]-Glucagon;
N{Epsilon-24}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Arg12,Lys24,Leu27]-Glucagon;
N{Epsilon-24}-[(4S)-4-carboxy-4-[[2-[2-[2-[[2-[2-[2-[[(4S)-4-carboxy-4-[[(4S)-4-carboxy-4-(17-carboxyheptadecanoylamino)butanoyl]amino]butanoyl]amino]ethoxy]ethoxy]acetyl]amino]ethoxy]ethoxy]acetyl]amino]butanoyl]-[Lys24,Leu27]-Glucagon;
N{Epsilon-24}-[2-[2-[2-[[2-[2-[2-[[(4S)-4-carboxy-4-[[(4S)-4-carboxy-4-[[(4S)-4-carboxy-4-(17-carboxyheptadecanoylamino)butanoyl]amino]butanoyl]amino]butanoyl]amino]ethoxy]ethoxy]acetyl]amino]ethoxy]ethoxy]acetyl]-[Lys24,Leu27]-Glucagon;
N{Epsilon-24}-[(4S)-4-carboxy-4-[[(4S)-4-carboxy-4-[[(4S)-4-carboxy-4-(17-carboxyheptadecanoylamino)butanoyl]amino]butanoyl]amino]butanoyl]-[Lys24,Leu27]-Glucagon;
N{Epsilon-25}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys25,Leu27]-Glucagon;
N{Epsilon-16}-[2-[2-[2-[[2-[2-[2-[[(4S)-4-carboxy-4-[[(4S)-4-carboxy-4-[[(4S)-4-carboxy-4-(17-carboxyheptadecanoylamino)butanoyl]amino]butanoyl]amino]butanoyl]amino]ethoxy]ethoxy]acetyl]amino]ethoxy]ethoxy]acetyl]-[Lys16,Leu27]-Glucagon;
N{Epsilon-16}-[(4S)-4-carboxy-4-[[2-[2-[2-[[2-[2-[2-[[(4S)-4-carboxy-4-[[(4S)-4-carboxy-4-(17-carboxyheptadecanoylamino)butanoyl]amino]butanoyl]amino]ethoxy]ethoxy]acetyl]amino]ethoxy]ethoxy]acetyl]amino]butanoyl]-[Lys16,Leu27]-Glucagon;
N{Epsilon-28}-[(4S)-4-carboxy-4-[[(4S)-4-carboxy-4-[[(4S)-4-carboxy-4-(17-carboxyheptadecanoylamino)butanoyl]amino]butanoyl]amino]butanoyl]-[Leu27,Lys28]-Glucagon;
N{Epsilon-12}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Leu27,Pro29]-Glucagon;
N{Epsilon-24}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys24,Leu27,Pro29]-Glucagon;
N{Epsilon-28}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Leu27,Lys28]-Glucagonyl-Pro;
N{Epsilon-12}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Leu27]-Glucagon;
N{Epsilon-24}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys24,Leu27]-Glucagonyl-Pro;
N{Epsilon-27}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys27,Pro29]-Glucagon;
{Epsilon-28}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Leu27,Lys28,Pro29]-Glucagon;
N{Epsilon-27}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Arg12,Lys27,Pro29]-Glucagon;
N{Epsilon-24}-[(2S)-4-carboxy-2-[[(2S)-4-carboxy-2-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys24,Leu27]-Glucagon;
N{Epsilon-24}-[(2S)-4-carboxy-2-[[(2S)-4-carboxy-2-[[2-[2-[2-[[2-[2-[2-[[(2S)-4-carboxy-2-(17-carboxyheptadecanoylamino)butanoyl]amino]ethoxy]ethoxy]acetyl]amino]ethoxy]ethoxy]acetyl]amino]butanoyl]amino]butanoyl]-[Lys24,Leu27]-Glucagon;
N{Epsilon-24}-[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]-[Lys24,Leu27]-Glucagon;
N{Epsilon-24}-[(4S)-4-carboxy-4-[[(4S)-4-carboxy-4-[[2-[2-[2-[[(4S)-4-carboxy-4-(17-carboxyheptadecanoylamino)butanoyl]amino]ethoxy]ethoxy]acetyl]amino]butanoyl]amino]butanoyl]-[Glu21,Lys24,Leu27,Ser28]-Glucagon;
N{Epsilon-24}-[(4S)-4-carboxy-4-[[(4S)-4-carboxy-4-[[2-[2-[2-[[(4S)-4-carboxy-4-(17-carboxyheptadecanoylamino)butanoyl]amino]ethoxy]ethoxy]acetyl]amino]butanoyl]amino]butanoyl]-[Glu9,Lys24,Leu27,Ser28]-Glucagon;
N{Epsilon-24}-[(4S)-4-carboxy-4-[[(4S)-4-carboxy-4-[[2-[2-[2-[[(4S)-4-carboxy-4-(17-carboxyheptadecanoylamino)butanoyl]amino]ethoxy]ethoxy]acetyl]amino]butanoyl]amino]butanoyl]-[Glu20,Glu21,Lys24,Leu27,Ser28]-Glucagon;
N{Epsilon-24}-[(4S)-4-carboxy-4-[[(4S)-4-carboxy-4-[[2-[2-[2-[[2-[2-[2-[[(4S)-4-carboxy-4-(15-carboxypentadecanoylamino)butanoyl]amino]ethoxy]ethoxy]acetyl]amino]ethoxy]ethoxy]acetyl]amino]butanoyl]amino]butanoyl]-[Lys24,Leu27]-Glucagon;
N{Epsilon-24}-[(4S)-4-carboxy-4-[[(4S)-4-carboxy-4-[[2-[2-[2-[[2-[2-[2-[[(4S)-4-carboxy-4-(11-carboxyundecanoylamino)butanoyl]amino]ethoxy]ethoxy]acetyl]amino]ethoxy]ethoxy]acetyl]amino]butanoyl]amino]butanoyl]-[Lys24,Leu27]-Glucagon;
N{Epsilon-24}-[(4S)-4-carboxy-4-[[(4S)-4-carboxy-4-[[2-[2-[2-[[2-[2-[2-[[(4S)-4-carboxy-4-(13-carboxytridecanoylamino)butanoyl]amino]ethoxy]ethoxy]acetyl]amino]ethoxy]ethoxy]acetyl]amino]butanoyl]amino]butanoyl]-[Lys24,Leu27]-Glucagon;
N{Epsilon-24}-[(4S)-4-carboxy-4-[[(4S)-4-carboxy-4-[[2-[2-[2-[[2-[2-[2-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]ethoxy]ethoxy]acetyl]amino]ethoxy]ethoxy]acetyl]amino]butanoyl]amino]butanoyl]-[Lys24,Leu27]-Glucagon;
N{Epsilon-20}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys20,Leu27]-Glucagon;
N{Epsilon-24}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[D-Phe4,Lys24,Leu27,Ser28]-Glucagon;
N{Epsilon-16}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys16,Glu21,Arg25,Leu27]-Glucagon;
N{Epsilon-24}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Glu20,Lys24,Leu27,Ser28]-Glucagon;
N{Epsilon-24}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys24,Gln27]-Glucagon;
N{Epsilon-24}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys24,Glu27]-Glucagon;
N{Epsilon-24}-[(4S)-4-carboxy-4-[[(4S)-4-carboxy-4-[[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]amino]butanoyl]amino]butanoyl]-[Lys24,Leu27]-Glucagon;
N{Epsilon-24}-[(4S)-4-carboxy-4-[[(4S)-4-carboxy-4-[[2-[2-[2-[[2-[2-[2-[[(4S)-4-carboxy-4-(7-carboxyheptanoylamino)butanoyl]amino]ethoxy]ethoxy]acetyl]amino]ethoxy]ethoxy]acetyl]amino]butanoyl]amino]butanoyl]-[Lys24,Leu27]-Glucagon;
N{Alpha}([His24, Leu27]-Glucagonyl)-N{Epsilon}[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]Lys;
N{Epsilon-24}-[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]-[Lys24,Glu27]-Glucagon;
N{Alpha}([Acb2]-Glucagonyl)-N{Epsilon}[(4S)-4-[[(4S)-4-[[(2R)-6-amino-2-[[(4S)-4-carboxy-4-(hexadecanoylamino)butanoyl]amino]hexanoyl]amino]-4-carboxybutanoyl]amino]-4-carboxybutanoyl]Lys amide;
N{Alpha}([Aib2]-Glucagonyl)-N{Epsilon}[(4S)-4-[[(4S)-4-[[(2R)-6-amino-2-[[(4S)-4-carboxy-4-(hexadecanoylamino)butanoyl]amino]hexanoyl]amino]-4-carboxybutanoyl]amino]-4-carboxybutanoyl]Lys amide;
   The amino acid abbreviations used in the present context have the following meanings:

| 3-letter | 1-letter | |
|---|---|---|
| AA | AA | |
| code | code | Name or structure |
| Ado | | 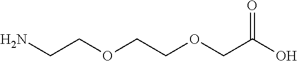 [SMILES: NCCOCCOCC(=O)O] |
| Aib | | 2-Aminoisobutyric acid |
| Ala | A | Alanine |
| Asn | N | Asparagine |
| Asp | D | Aspartic acid |
| β-Asp | | 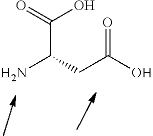 |
| | | α-nitrogen and β-carboxy group |
| | | form the amide bonds |
| | | to the two neighboring residues |
| Arg | R | Arginine |
| Cit | | Citrulline |
| Cys | C | Cysteine |
| Gln | Q | Glutamine |
| Glu | E | Glutamic acid |
| γ-Glu | | 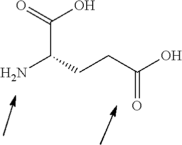 |
| | | α-nitrogen and γ-carboxy group |
| | | form the amide bonds |
| | | to the two neighboring residues |
| Gly | G | Glycine |
| His | H | Histidine |
| Hyp | | 4-hydroxyproline |
| Ile | I | Isoleucine |
| Leu | L | Leucine |
| Lys | K | Lysine |
| ε-Lys | | 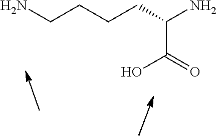 |
| | | ε-nitrogen and α-carboxy group |
| | | form the amide bonds |
| | | to the two neighboring residues |
| Met | M | Methionine |
| Met(O) | | 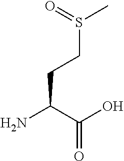 |
| Orn | | Ornithine |
| Phe | F | Phenylalanine |
| Pro | P | Proline |
| Ser | S | Serine |
| Thr | T | Threonine |
| Tyr | Y | Tyrosine |
| p(Tyr) | | 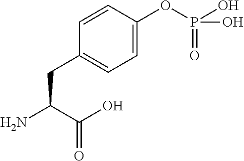 [SMILES: N[C@@H](Cc1ccc(OP(=O)(O)O)cc1)C(=O)O] |
| Trp | W | Tryptophan |
| Val | V | Valine |

Amino acid abbreviations beginning with D-followed by a three letter code, such as D-Ser, D-His and so on, refer to the D-enantiomer of the corresponding amino acid, for example D-serine, D-histidine and so on.

### Further Embodiments of the Present Invention are

1C. A glucagon peptide comprising a substituent comprising between three and ten negatively charged moieties, attached to the side chain of an amino acid of said glucagon peptide or a pharmaceutically acceptable salt, amide, carboxylic acid or prodrug thereof, with the proviso that said substituent does not comprise a lipophilic moiety.

2C. The glucagon peptide according to embodiment 1C, wherein said substituent is attached to the side chain of an amino acid in positions X₁₀, X₁₂, X₁₆, X₁₇, X₁₈, X₂₀, X₂₁, X₂₄, X₂₅, X₂₇, X₂₈, X₂₉, and/or X₃₀ of said glucagon peptide.

3C. The glucagon peptide according to embodiments 1C-2C, wherein said substituent is attached to the side chain of an amino acid in position X₂₄ of said glucagon peptide.

4C. The glucagon peptide according to any one of embodiments 1C-3C, wherein X₂₄ represents Lys.

5C. The glucagon peptide according to any one of embodiments 1C-4C, wherein said glucagon peptide comprises up to 15 amino acid residue substitutions in said glucagon peptide and wherein said substitutions may be in the following amino acid positions: X₂, X₃, X₄, X₉, X₁₀, X₁₂, X₁₅, X₁₆, X₁₇, X₁₈, X₂₀, X₂₁, X₂₄, X₂₅, X₂₇, X₂₈, X₂₉ and/or X₃₀.

6C. A glucagon peptide according to any one of embodiments 1C-5C, wherein said substituent has the formula II:

Y₁-Y₂-Y₃-Y₄-Y₅-Y₆-Y₇-Y₈-Y₉-Y₁₀ [II]

wherein
Y₁ represents a proteinogenic amino acid or a structure of the formula iv, or a structure of the formula v or is absent
Y₂, Y₃, Y₄, Y₅, Y₆, Y₇, Y₈ and Y₉ is individually represented by the structures i, ii, iii or is absent and Y₁₀ is represented by the structure vi connected via an amide bond or is absent

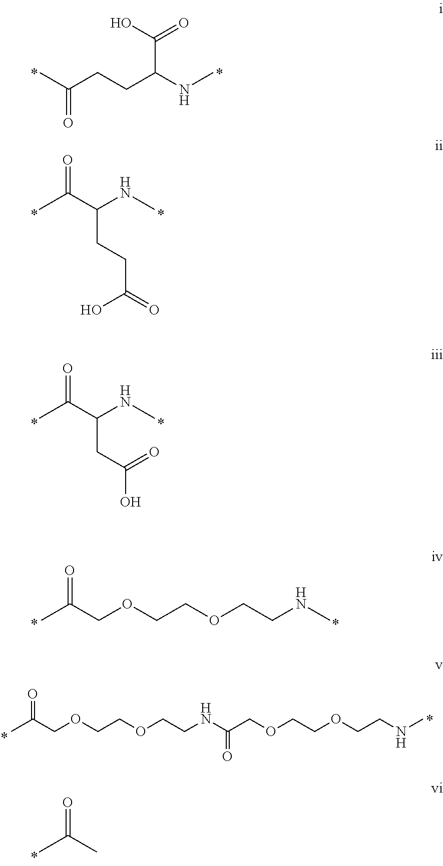

wherein * represents the attachment point
provided that Y₁-Y₂-Y₃-Y₄-Y₅-Y₆-Y₇-Y₈-Y₉-Y₁₀ contains at least three negative charged moieties and wherein each amino acid i, ii and iii independently has the stereochemistry L or D.

7C. The glucagon peptide according to any one of embodiments 1C-6C, selected from the group consisting of: Chem.1, Chem.2, Chem.3, Chem.4, Chem.5, Chem.6, Chem.7, Chem.8, Chem.9, Chem.10, Chem.11, Chem.12, Chem.13, Chem.14, Chem.15, Chem.16, Chem.17, Chem.18, Chem.19, Chem.20, Chem.21, Chem.22, Chem.23, Chem.24, Chem.25, Chem.26, Chem.27, Chem.28, Chem.29, Chem.30, Chem.31, Chem.32, Chem.33, Chem.34, Chem.35, Chem.36, Chem.37, Chem.38, Chem.39, Chem.40, Chem.41, Chem.42, Chem.43, Chem.44, Chem.45, Chem.46, Chem.47 and Chem.48, Chem.49, Chem.50, Chem.51, Chem.52, Chem.53, Chem.54, Chem.55, Chem.56, Chem.57, Chem.58, Chem.59 Chem.60, Chem.61, Chem.62, Chem.63, Chem.64, Chem.65, Chem.66, Chem.67, Chem.68 and Chem.69.

8C. A pharmaceutical composition comprising a glucagon peptide according to any one of embodiments 1C-7C.

9C. The pharmaceutical composition according to embodiment 8C, further comprising one or more additional therapeutically active compounds or substances.

10C. The pharmaceutical composition according to any one of embodiments 8C-9C, which is suited for parenteral administration.

11C. A glucagon peptide according to any of any one of embodiments 1C-7C, for use in therapy.

12C. Use of a glucagon peptide according to any one of the embodiments 1C-7C, for the preparation of a medicament.

13C. Use of a glucagon peptide according to any one of embodiments 1C-7C, for the preparation of a medicament for the treatment or prevention of hyperglycemia, type 2 diabetes, impaired glucose tolerance, type 1 diabetes and obesity.

14C. Use of a glucagon peptide according to any one of the embodiments 1C-7C, for the preparation of a medicament for delaying or preventing disease progression in type 2 diabetes, treating obesity or preventing overweight, for decreasing food intake, increase energy expenditure, reducing body weight, delaying the progression from impaired glucose tolerance (IGT) to type 2 diabetes; delaying the progression from type 2 diabetes to insulin-requiring diabetes; regulating appetite; inducing satiety; preventing weight regain after successful weight loss; treating a disease or state related to overweight or obesity; treating bulimia; treating binge-eating; treating atherosclerosis, hypertension, type 2 diabetes, IGT, dyslipidemia, coronary heart disease, hepatic steatosis, treatment of beta-blocker poisoning, use for inhibition of the motility of the gastrointestinal tract, useful in connection with investigations of the gastrointestinal tract using techniques such as x-ray, CT- and NMR-scanning.

15C. Use of a glucagon peptide according to any one of the embodiments 1C-7C, for the preparation of a medicament for treatment or prevention of hypoglycemia, insulin induced hypoglycemia, reactive hypoglycemia, diabetic hypoglycemia, non-diabetic hypoglycemia, fasting hypoglycemia, drug-induced hypoglycemia, gastric by-pass induced hypoglycemia, hypoglycemia in pregnancy, alcohol induced hypoglycemia, insulinoma and Von Girkes disease.

### Pharmaceutical Compositions

Pharmaceutical compositions containing a compound according to the present invention may be prepared by conventional techniques, e.g. as described in Remington's Pharmaceutical Sciences, 1985 or in Remington: The Science and Practice of Pharmacy, 19^{th} edition, 1995.

As already mentioned, one aspect of the present invention is to provide a pharmaceutical formulation comprising a compound according to the present invention which is present in a concentration from about 0.01 mg/mL to about 25 mg/mL, such as from about 0.1 mg/mL to about 5 mg/mL and from about 2 mg/mL to about 5 mg/mL, and wherein said formulation has a pH from 2.0 to 10.0. The pharmaceutical formulation may comprise a compound according to the present invention which is present in a concentration from about 0.1 mg/ml to about 50 mg/ml, and wherein said formulation has a pH from 2.0 to 10.0. The formulation may further comprise a buffer system, preservative(s), isotonicity agent(s), chelating agent(s), stabilizers and surfactants. In one embodiment of the invention the pharmaceutical formulation is an aqueous formulation, i.e. formulation comprising water. Such formulation is typically a solution or a suspension. In a further embodiment of the invention the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another embodiment the pharmaceutical formulation is a freeze-dried formulation, whereto the physician or the patient adds solvents and/or diluents prior to use.

In another embodiment the pharmaceutical formulation is a dried formulation (e.g. freeze-dried or spray-dried) ready for use without any prior dissolution.

In a further aspect the invention relates to a pharmaceutical formulation comprising an aqueous solution of a compound according to the present invention, and a buffer, wherein said compound is present in a concentration from 0.1 mg/ml or above, and wherein said formulation has a pH from about 2.0 to about 10.0.

In a further aspect the invention relates to a pharmaceutical formulation comprising an aqueous solution of a compound according to the present invention, and a buffer, wherein said compound is present in a concentration from 0.1 mg/ml or above, and wherein said formulation has a pH from about 7.0 to about 8.5. In a further aspects of the invention said formulation has a pH from about 6.0 to about 7.5 or from about 5.0 to about 7.5

In a another embodiment of the invention the pH of the formulation is selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0. Preferably, the pH of the formulation is at least 1 pH unit from the isoelectric point of the compound according to the present invention, even more preferable the pH of the formulation is at least 2 pH unit from the isoelectric point of the compound according to the present invention.

In a further embodiment of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethane, hepes, bicine, tricine, malic acid, succinate, maleic acid, fumaric acid, tartaric acid, aspartic acid or mixtures thereof. Each one of these specific buffers constitutes an alternative embodiment of the invention.

In a further embodiment of the invention the formulation further comprises a pharmaceutically acceptable preservative. In a further embodiment of the invention the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, ethanol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In a further embodiment of the invention the preservative is present in a concentration from 0.1 mg/ml to 30 mg/ml. In a further embodiment of the invention the preservative is present in a concentration from 0.1 mg/ml to 20 mg/ml. In a further embodiment of the invention the preservative is present in a concentration from 0.1 mg/ml to 5 mg/ml. In a further embodiment of the invention the preservative is present in a concentration from 5 mg/ml to 10 mg/ml. In a further embodiment of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 19^{th} edition, 1995.

In a further embodiment of the invention the formulation further comprises an isotonic agent. In a further embodiment of the invention the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol)polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C4-C8 hydrocarbon having at least one —OH group and includes, for example, mannitol, sorbitol, inositol, galacititol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods of the invention. In one embodiment, the sugar or sugar alcohol concentration is between about 1 mg/ml and about 150 mg/ml. In a further embodiment of the invention the isotonic agent is present in a concentration from 1 mg/ml to 50 mg/ml. In a further embodiment of the invention the isotonic agent is present in a concentration from 1 mg/ml to 7 mg/ml. In a further embodiment of the invention the isotonic agent is present in a concentration from 8 mg/ml to 24 mg/ml. In a further embodiment of the invention the isotonic agent is present in a concentration from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 19^{th} edition, 1995.

In a further embodiment of the invention the formulation further comprises a chelating agent. In a further embodiment of the invention the chelating agent is selected from salts of ethylenediaminetetraacetic acid (EDTA), citric acid, and aspartic acid, and mixtures thereof. In a further embodiment of the invention the chelating agent is present in a concentration from 0.1 mg/ml to 5 mg/ml. In a further embodiment of the invention the chelating agent is present in a concentration from 0.1 mg/ml to 2 mg/ml. In a further embodiment of the invention the chelating agent is present in a concentration from 2 mg/ml to 5 mg/ml. Each one of these specific chelating agents constitutes an alternative embodiment of the invention. The use of a chelating agent in pharmaceutical compositions is well-known to the skilled person. For convenience reference is made to Remington: The Science and Practice of Pharmacy, 19^{th} edition, 1995.

In a further embodiment of the invention the formulation further comprises a stabiliser. The use of a stabilizer in pharmaceutical compositions is well-known to the skilled person. For convenience reference is made to Remington: The Science and Practice of Pharmacy, 19^{th} edition, 1995.

More particularly, compositions of the invention are stabilized liquid pharmaceutical compositions whose therapeutically active components include a polypeptide that possibly exhibits aggregate formation during storage in liquid pharmaceutical formulations. By “aggregate formation” is intended a physical interaction between the polypeptide molecules that results in formation of oligomers, which may remain soluble, or large visible aggregates that precipitate from the solution. By “during storage” is intended a liquid pharmaceutical composition or formulation once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or formulation is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

The pharmaceutical compositions of the invention may further comprise an amount of an amino acid base sufficient to decrease aggregate formation by the polypeptide during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids used for preparing the compositions of the invention are those carrying a charged side chain, such as arginine, lysine, aspartic acid, and glutamic acid. In one embodiment, the amino acid used for preparing the compositions of the invention is glycine. Any stereoisomer (i.e. L or D) of a particular amino acid (e.g. methionine, histidine, imidazole, arginine, lysine, isoleucine, aspartic acid, tryptophan, threonine and mixtures thereof) or combinations of these stereoisomers, may be present in the pharmaceutical compositions of the invention so long as the particular amino acid is present either in its free base form or its salt form. In one embodiment the L-stereoisomer is used. Compositions of the invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical compositions of the invention. Suitable arginine analogues include, for example, aminoguanidine, ornithine and N-monoethyl L-arginine, suitable methionine analogues include ethionine and buthionine and suitable cystein analogues include S-methyl-L cystein. As with the other amino acids, the amino acid analogues are incorporated into the compositions in either their free base form or their salt form. In a further embodiment of the invention the amino acids or amino acid analogues are used in a concentration, which is sufficient to prevent or delay aggregation of the protein.

In a further embodiment of the invention methionine (or other sulphuric amino acids or amino acid analogous) may be added to inhibit oxidation of methionine residues to methionine sulfoxide when the polypeptide acting as the therapeutic agent is a polypeptide comprising at least one methionine residue susceptible to such oxidation. By “inhibit” is intended minimal accumulation of methionine oxidized species over time. Inhibiting methionine oxidation results in greater retention of the polypeptide in its proper molecular form. Any stereoisomer of methionine (L, D or a mixture thereof) can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be achieved by adding methionine such that the ratio of methionine added to methionine residues ranges from about 1:1 to about 1000:1, such as 10:1 to about 100:1.

In a further embodiment of the invention the formulation further comprises a stabiliser selected from the group of high molecular weight polymers or low molecular compounds. In a further embodiment of the invention the stabilizer is selected from polyethylene glycol (e.g. PEG 3350), polyvinylalcohol (PVA), polyvinylpyrrolidone, carboxy-/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment of the invention.

The pharmaceutical compositions may also comprise additional stabilizing agents, which further enhance stability of a therapeutically active polypeptide therein. Stabilizing agents of particular interest to the present invention include, but are not limited to, methionine and EDTA, which protect the polypeptide against methionine oxidation, and a nonionic surfactant, which protects the polypeptide against aggregation associated with freeze-thawing or mechanical shearing.

In a further embodiment of the invention the formulation further comprises a surfactant. In a further embodiment of the invention the surfactant is selected from a detergent, ethoxylated castor oil, polyglycolyzed glycerides, acetylated monoglycerides, sorbitan fatty acid esters, polyoxypropylene-polyoxyethylene block polymers (eg. poloxamers such as Pluronic® F68, poloxamer 188 and 407, Triton X-100), polyoxyethylene sorbitan fatty acid esters, starshaped PEO, polyoxyethylene and polyethylene derivatives such as alkylated and alkoxylated derivatives (tweens, e.g. Tween-20, Tween-40, Tween-80 and Brij-35), polyoxyethylene hydroxystearate, monoglycerides or ethoxylated derivatives thereof, diglycerides or polyoxyethylene derivatives thereof, alcohols, glycerol, lecitins and phospholipids (eg. phosphatidyl serine, phosphatidyl choline, phosphatidyl ethanolamine, phosphatidyl inositol, diphosphatidyl glycerol and sphingomyelin), derivates of phospholipids (eg. dipalmitoyl phosphatidic acid) and lysophospholipids (eg. palmitoyl lysophosphatidyl-L-serine and 1-acyl-sn-glycero-3-phosphate esters of ethanolamine, choline, serine or threonine) and alkyl, alkoxyl (alkyl ester), alkoxy (alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (eg. galactopyransoide), sphingoglycolipids (eg. ceramides, gangliosides), dodecylphosphocholine, hen egg lysolecithin, fusidic acid derivatives—(e.g. sodium tauro-dihydrofusidate etc.), long-chain fatty acids and salts thereof C6-C12 (eg. oleic acid and caprylic acid), acylcarnitines and derivatives, N^{α}-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N^{α}-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulfate or sodium lauryl sulfate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quarternary ammonium bases) (e.g. cetyl-trimethylammonium bromide, cetylpyridinium chloride), non-ionic surfactants (eg. Dodecyl 0-D-glucopyranoside), poloxamines (eg. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative embodiment of the invention.

The use of a surfactant in pharmaceutical compositions is well-known to the skilled person. For convenience reference is made to Remington: The Science and Practice of Pharmacy, 19^{th} edition, 1995.

Additional ingredients may also be present in the pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatin or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Pharmaceutical compositions containing a compound according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the compound, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

Compositions of the current invention are useful in the formulation of solids, semisolids, powder and solutions for pulmonary administration of the compound, using, for example a metered dose inhaler, dry powder inhaler and a nebulizer, all being devices well known to those skilled in the art.

Compositions of the current invention are specifically useful in the formulation of controlled, sustained, protracting, retarded, and slow release drug delivery systems. More specifically, but not limited to, compositions are useful in formulation of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles,

Methods to produce controlled release systems useful for compositions of the current invention include, but are not limited to, crystallization, condensation, co-cystallization, precipitation, co-precipitation, emulsification, dispersion, high pressure homogenization, encapsulation, spray drying, microencapsulation, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Formulation and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the compound according to the present invention in the form of a nasal or pulmonal spray. As a still further option, the pharmaceutical compositions containing the compound of the invention can also be adapted to transdermal administration, e.g. by needle-free injection or from a patch, optionally an iontophoretic patch, or transmucosal, e.g. buccal, administration.

The term “stabilized formulation” refers to a formulation with increased physical stability, increased chemical stability or increased physical and chemical stability.

The term “physical stability” of the protein formulation as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein formulations is evaluated by means of visual inspection and/or turbidity measurements after exposing the formulation filled in suitable containers (e.g. cartridges or vials) to mechanical/physical stress (e.g. agitation) at different temperatures for various time periods. Visual inspection of the formulations is performed in a sharp focused light with a dark background. The turbidity of the formulation is characterized by a visual score ranking the degree of turbidity for instance on a scale from 0 to 3 (a formulation showing no turbidity corresponds to a visual score 0, and a formulation showing visual turbidity in daylight corresponds to visual score 3). A formulation is classified physical unstable with respect to protein aggregation, when it shows visual turbidity in daylight. Alternatively, the turbidity of the formulation can be evaluated by simple turbidity measurements well-known to the skilled person. Physical stability of the aqueous protein formulations can also be evaluated by using a spectroscopic agent or probe of the conformational status of the protein. The probe is preferably a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as antrhacene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the protein formulation as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein formulation as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation (of for instance methionine residues) can be mentioned as another variant of chemical degradation. The chemical stability of the protein formulation can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

Hence, as outlined above, a “stabilized formulation” refers to a formulation with increased physical stability, increased chemical stability or increased physical and chemical stability. In general, a formulation must be stable during use and storage (in compliance with recommended use and storage conditions) until the expiration date is reached.

In one embodiment of the invention the pharmaceutical formulation comprising the compound according to the present invention is stable for more than 6 weeks of usage and for more than 3 years of storage.

In another embodiment of the invention the pharmaceutical formulation comprising the compound according to the present invention is stable for more than 4 weeks of usage and for more than 3 years of storage.

In a further embodiment of the invention the pharmaceutical formulation comprising the compound according to the present invention is stable for more than 4 weeks of usage and for more than two years of storage.

In an even further embodiment of the invention the pharmaceutical formulation comprising the compound is stable for more than 2 weeks of usage and for more than two years of storage.

In an even further embodiment of the invention the pharmaceutical formulation comprising the compound is stable for more than 24 weeks of usage and for more than 18 months of storage.

Pharmaceutical compositions containing a glucagon peptide according to the present invention may be administered parenterally to patients in need of such a treatment. Parenteral administration may be performed by subcutaneous, intramuscular or intravenous injection by means of a syringe, optionally a pen-like syringe. Alternatively, parenteral administration can be performed by means of an infusion pump. A further option is a composition which may be a powder or a liquid for the administration of the glucagon peptide in the form of a nasal or pulmonal spray. As a still further option, the glucagon peptides of the invention can also be administered transdermally, e.g. from a patch, optionally a iontophoretic patch, or transmucosally, e.g. bucally.

Thus, the injectable compositions of the glucagon peptide of the present invention can be prepared using the conventional techniques of the pharmaceutical industry which involves dissolving and mixing the ingredients as appropriate to give the desired end product.

According to one embodiment of the present invention, the glucagon peptide is provided in the form of a composition suitable for administration by injection. Such a composition can either be an injectable solution ready for use or it can be an amount of a solid composition, e.g. a lyophilised product, which has to be dissolved in a solvent before it can be injected.

The glucagon peptides of this invention can be used in the treatment of various diseases. The particular glucagon peptide to be used and the optimal dose level for any patient will depend on the disease to be treated and on a variety of factors including the efficacy of the specific peptide derivative employed, the age, body weight, physical activity, and diet of the patient, on a possible combination with other drugs, and on the severity of the case. It is recommended that the dosage of the glucagon peptide of this invention be determined for each individual patient by those skilled in the art.

In particular, it is envisaged that the glucagon peptide will be useful for the preparation of a medicament with a protracted profile of action for the treatment of non-insulin dependent diabetes mellitus and/or for the treatment of obesity.

In another aspect the present invention relates to the use of a compound according to the invention for the preparation of a medicament.

In one embodiment the present invention relates to the use of a compound according to the invention for the preparation of a medicament for the treatment of hyperglycemia, type 2 diabetes, impaired glucose tolerance, type 1 diabetes, obesity, hypertension, syndrome X, dyslipidemia, β-cell apoptosis, β-cell deficiency, myocardial infarction, inflammatory bowel syndrome, dyspepsia, cognitive disorders, e.g. cognitive enhancing, neuroprotection, atheroschlerosis, coronary heart disease and other cardiovascular disorders.

In another embodiment the present invention relates to the use of a compound according to the invention for the preparation of a medicament for the treatment of small bowel syndrome, inflammatory bowel syndrome or Crohns disease.

In another embodiment the present invention relates to the use of a compound according to the invention for the preparation of a medicament for the treatment of hyperglycemia, type 1 diabetes, type 2 diabetes or β-cell deficiency.

The treatment with a compound according to the present invention may also be combined with combined with a second or more pharmacologically active substances, e.g. selected from antidiabetic agents, antiobesity agents, appetite regulating agents, antihypertensive agents, agents for the treatment and/or prevention of complications resulting from or associated with diabetes and agents for the treatment and/or prevention of complications and disorders resulting from or associated with obesity. In the present context the expression “antidiabetic agent” includes compounds for the treatment and/or prophylaxis of insulin resistance and diseases wherein insulin resistance is the pathophysiological mechanism.

Examples of these pharmacologically active substances are: Insulin, GLP-1 agonists, sulphonylureas (e.g. tolbutamide, glibenclamide, glipizide and gliclazide), biguanides e.g. metformin, meglitinides, glucosidase inhibitors (e.g. acorbose), glucagon antagonists, DPP-IV (dipeptidyl peptidase-IV) inhibitors, inhibitors of hepatic enzymes involved in stimulation of gluconeogenesis and/or glycogenolysis, glucose uptake modulators, thiazolidinediones such as troglitazone and ciglitazone, compounds modifying the lipid metabolism such as antihyperlipidemic agents as HMG CoA inhibitors (statins), compounds lowering food intake, RXR agonists and agents acting on the ATP-dependent potassium channel of the β-cells, e.g. glibenclamide, glipizide, gliclazide and repaglinide; Cholestyramine, colestipol, clofibrate, gemfibrozil, lovastatin, pravastatin, simvastatin, probucol, dextrothyroxine, neteglinide, repaglinide; β-blockers such as alprenolol, atenolol, timolol, pindolol, propranolol and metoprolol, ACE (angiotensin converting enzyme) inhibitors such as benazepril, captopril, enalapril, fosinopril, lisinopril, alatriopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin; CART (cocaine amphetamine regulated transcript) agonists, NPY (neuropeptide Y) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, β3 agonists, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, RXR (retinoid X receptor) modulators, TR β agonists; histamine H3 antagonists.

It should be understood that any suitable combination of the compounds according to the invention with one or more of the above-mentioned compounds and optionally one or more further pharmacologically active substances are considered to be within the scope of the present invention.

The present invention is further illustrated by the following examples which, however, are not to be construed as limiting the scope of protection. The features disclosed in the foregoing description and in the following examples may, both separately and in any combination thereof, be material for realizing the invention in diverse forms thereof.

### EXAMPLES

### List of Abbreviations

BOC: tert-Butyl oxycarbonyl
DCM: Dichloromethane
DIC: Diisopropylcarbodiimide
Fmoc: 9-fluorenylmethyloxycarbonyl
HOAt: 1-hydroxy-7-azabenzotriazole
HPLC: High Performance Liquid Chromatography
LCMS: Liquid Chromatography Mass Spectroscopy
MeCN: Acetonitrile
Mtt: 4-Methyltrityl
NMP: N-methyl pyrrolidone
Oxyma Pure: Cyano-[hydroxyimino]-acetic acid ethyl ester
RP: Reverse Phase
RP-HPLC: Reverse Phase High Performance Liquid Chromatography
RT: Room Temperature
Rt: Retention time
SPPS: Solid Phase Peptide Synthesis
TFA: Trifluoroacetic acid
TIPS: Triisopropylsilane
UPLC: Ultra Performance Liquid Chromatography

### General Methods

This section relates to methods for synthesising resin bound peptide (SPPS methods, including methods for de-protection of amino acids, methods for cleaving the peptide from the resin, and for its purification), as well as methods for detecting and characterising the resulting peptide (LCMS and UPLC methods).

### SPPS General Methods

The Fmoc-protected amino acid derivatives used were the standard recommended: Fmoc-Ala-OH, Fmoc-Arg(Pbf)-OH, Fmoc-Asn(Trt)-OH, Fmoc-Asp(OtBu)-OH, Fmoc-Cys(Trt)-OH, Fmoc-Gln(Trt)-OH, Fmoc-Glu(OtBu)-OH, Fmoc-Gly-OH, Fmoc-His(Trt)-OH, Fmoc-Ile-OH, Fmoc-Leu-OH, Fmoc-Lys(BOC)-OH, Fmoc-Met-OH, Fmoc-Phe-OH, Fmoc-Pro-OH, Fmoc-Ser(tBu)-OH, Fmoc-Thr(tBu)-OH, Fmoc-Trp(BOC)-OH, Fmoc-Tyr(tBu)-OH and Fmoc-Val-OH, Fmoc-Lys(Mtt)-OH supplied from e.g. Anaspec, Bachem, Iris Biotech, or NovabioChem.

SPPS were performed using Fmoc based chemistry on a Prelude Solid Phase Peptide Synthesizer from Protein Technologies (Tucson, Ariz. 85714 U.S.A.). A suitable resin for the preparation of C-terminal carboxylic acids is a pre-loaded, low-load Wang resin available from NovabioChem (e.g. low load fmoc-Thr(tBu)-Wang resin, LL, 0.27 mmol/g). A suitable resin for the synthesis of glucagon analogues with a C-terminal amide is PAL-ChemMatrix resin available from Matrix-Innovation. The N-terminal alpha amino group was protected with Boc. When histidine was used as the N-terminal amino acid Boc-His(Trt)-OH was used.

Fmoc-deprotection was achieved with 20% piperidine in NMP for 2×3 min. The coupling chemistry was DIC/HOAt/collidine or DIC/Oxyma Pure/collidine in NMP. Amino acid/HOAt or amino acid/OXYMA solutions (0.3 M/0.3 M in NMP at a molar excess of 3-10 fold) were added to the resin followed by the same molar equivalent of DIC (3 M in NMP) followed by collidine (3 M in NMP). For example, the following amounts of 0.3 M amino acid/HOAt solution were used per coupling for the following scale reactions: Scale/ml, 0.05 mmol/1.5 mL, 0.10 mmol/3.0 mL, 0.25 mmol/7.5 mL. Coupling time was either 2×30 min or 1×240 min.

The introduction of a substituent on the ε-nitrogen of a lysine was achived using a Lysine protected with Mtt (Fmoc-Lys(Mtt)-OH). The Mtt group was removed by washing the resin with HFIP/DCM (75:25) (2×2 min), washed with DCM and suspending the resin in HFIP/DCM (75:25)(2×20 min) and subsequently washed in sequence with Piperidine/NMP (20:80), DCM(1×), NMP(1×), DCM(1×), NMP(1×).

Likewise when the side-chain is present on an ornithine sidechain the delta aminogroup of the ornithine to be acylated is protected with Mtt (e.g. Fmoc-Orn(Mtt)-OH. Alternatively the ε-nitrogen of a lysine could be protected with an ivDde group (Fmoc-Lys(ivDde)-OH). The delta aminogroup of an ornitine can likewise be protected with an ivDde group (Fmoc-Orn(ivDde)-OH). The incorporation of gamma-Glu moieties in the substituent were achieved by coupling with the amino acid Fmoc-Glu-OtBu. Introduction of ε-Lys in the substituent was achieved using Boc-Lys(fmoc)-OH.

Introduction of each moiety in the side-chain was achieved using prolonged coupling time (1×6 hours) followed by capping with acetic anhydride or alternatively acetic acid/DIC/HOAt/collidine. Acetylation of the terminal nitrogen on the substituent was achieved using acetic anhydride (10 eq.) and collidine (20 eq.) in NMP. The introduction of a succinoyl moiety was achieved with succinic anhydride. The introduction of other C₂₋₆ acyl moieties was achieved using the corresponding carboxylic acid/DIC/Oxyma Pure/collidine (10 eq. each).

Cleavage from the Resin

After synthesis the resin was washed with DCM, and the peptide was cleaved from the resin by a 2-3 hour treatment with TFA/TIS/water (95/2.5/2.5) followed by precipitation with diethylether. The precipitate was washed with diethylether.

### Purification and Quantification

The crude peptide is dissolved in a suitable mixture of water and MeCN such as water/MeCN (4:1) and purified by reversed-phase preparative HPLC (Waters Deltaprep 4000 or Gilson) on a column containing C18-silica gel. Elution is performed with an increasing gradient of MeCN in water containing 0.1% TFA. Relevant fractions are checked by analytical HPLC or UPLC. Fractions containing the pure target peptide are mixed and concentrated under reduced pressure. The resulting solution is analyzed (HPLC, LCMS) and the product is quantified using a chemiluminescent nitrogen specific HPLC detector (Antek 8060 HPLC-CLND) or by measuring UV-absorption at 280 nm. The product is dispensed into glass vials. The vials are capped with Millipore glassfibre prefilters. Freeze-drying affords the peptide trifluoroacetate as a white solid.

### Methods for Detection and Characterization

### LCMS Methods

### Method: LCMS_{—}2

A Perkin Elmer Sciex API 3000 mass spectrometer was used to identify the mass of the sample after elution from a Perkin Elmer Series 200 HPLC system. Eluents: A: 0.05% Trifluoro acetic acid in water; B: 0.05% Trifluoro acetic acid in acetonitrile. Column: Waters Xterra MS C-18×3 mm id 5 μm. Gradient: 5%-90% B over 7.5 min at 1.5 ml/mln.

### Method: LCMS_{—}4

LCMS_{—}4 was performed on a setup consisting of Waters Acquity UPLC system and LCT Premier XE mass spectrometer from Micromass. Eluents: A: 0.1% Formic acid in water B: 0.1% Formic acid in acetonitrile The analysis was performed at RT by injecting an appropriate volume of the sample (preferably 2-10 μl) onto the column which was eluted with a gradient of A and B. The UPLC conditions, detector settings and mass spectrometer settings were: Column: Waters Acquity UPLC BEH, C-18, 1.7 μm, 2.1 mm×50 mm. Gradient: Linear 5%-95% acetonitrile during 4.0 min (alternatively 8.0 min) at 0.4 ml/mln. Detection: 214 nm (analogue output from TUV (Tunable UV detector)) MS ionisation mode: API-ES Scan: 100-2000 amu (alternatively 500-2000 amu), step 0.1 amu.

### Method: LCMS_AP

A Micromass Quatro micro API mass spectrometer was used to identify the mass of the sample after elution from a HPLC system composed of Waters2525 binary gradient modul, Waters2767 sample manager, Waters 2996 Photodiode Array Detector and Waters 2420 ELS Detector. Eluents: A: 0.1% Trifluoro acetic acid in water; B: 0.1% Trifluoro acetic acid in acetonitrile. Column: Phenomenex Synergi MAXRP, 4 um, 75×4.6 mm. Gradient: 5%-95% B over 7 min at 1.0 ml/mln.

### UPLC Methods

### Method 04_A3_{—}1

UPLC (method 04_A3_{—}1): The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C.

The UPLC system was connected to two eluent reservoirs containing:

A: 90% H2O, 10% CH3CN, 0.25 M ammonium bicarbonate

B: 70% CH3CN, 30% H₂O

The following linear gradient was used: 75% A, 25% B to 45% A, 55% B over 16 minutes at a flow-rate of 0.35 ml/mln.

Method 04_A4_{—}1

UPLC (method 04_A4_{—}1): The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C.

The UPLC system was connected to two eluent reservoirs containing:

A: 90% H2O, 10% CH3CN, 0.25 M ammonium bicarbonate

B: 70% CH3CN, 30% H₂O

The following linear gradient was used: 65% A, 35% B to 25% A, 65% B over 16 minutes at a flow-rate of 0.35 ml/mln.

Method: 04_A2_{—}1

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C. The UPLC system was connected to two eluent reservoirs containing: A: 90% H2O, 10% CH3CN, 0.25 M ammonium bicarbonate; B: 70% CH3CN, 30% H2O. The following linear gradient was used: 90% A, 10% B to 60% A, 40% B over 16 minutes at a flow-rate of 0.40 ml/mln.

Method: 04_A6_{—}1

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C. The UPLC system was connected to two eluent reservoirs containing: A: 10 mM TRIS, 15 mM ammonium sulphate, 80% H2O, 20%, pH 7.3; B: 80% CH3CN, 20% H2O. The following linear gradient was used: 95% A, 5% B to 10% A, 90% B over 16 minutes at a flow-rate of 0.35 ml/mln.

Method: 04_A7_{—}1

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C. The UPLC system was connected to two eluent reservoirs containing: A: 10 mM TRIS, 15 mM ammonium sulphate, 80% H2O, 20%, pH 7.3; B: 80% CH3CN, 20% H2O. The following linear gradient was used: 95% A, 5% B to 40% A, 60% B over 16 minutes at a flow-rate of 0.40 ml/mln.

Method: 04_A9_{—}1

The RP-analysis was performed using a Waters UPLC system fitted with a dual band detector. UV detections at 214 nm and 254 nm were collected using an ACQUITY UPLC BEH Shield RP18, C18, 1.7 um, 2.1 mm×150 mm column, 60° C. The UPLC system was connected to two eluent reservoirs containing: A: 200 mM Na2SO4+20 mM Na2HPO4+20 mM NaH2PO4 in 90% H₂O/10% CH3CN, pH 7.2; B: 70% CH₃CN, 30% H₂O. The following step gradient was used: 90% A, 10% B to 80% A, 20% B over 3 minutes, 80% A, 20% B to 50% A, 50% B over 17 minutes at a flow-rate of 0.40 ml/mln.

Method 05_B5_{—}1

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C.

The UPLC system was connected to two eluent reservoirs containing:

A: 0.2 M Na2SO4, 0.04 M H3PO4, 10% CH3CN (pH 3.5)

B: 70% CH3CN, 30% H₂O

The following linear gradient was used: 60% A, 40% B to 30% A, 70% B over 8 minutes at a flow-rate of 0.35 ml/mln.

Method: 05_B7_{—}1

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C. The UPLC system was connected to two eluent reservoirs containing: A: 0.2 M Na2SO4, 0.04 M H3PO4, 10% CH3CN (pH 3.5); B: 70% CH3CN, 30% H2O. The following linear gradient was used: 80% A, 20% B to 40% A, 60% B over 8 minutes at a flow-rate of 0.40 ml/mln.

Method: 05_B8_{—}1

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C. The UPLC system was connected to two eluent reservoirs containing: A: 0.2 M Na2SO4, 0.04 M H3PO4, 10% CH3CN (pH 3.5); B: 70% CH3CN, 30% H2O. The following linear gradient was used: 50% A, 50% B to 20% A, 80% B over 8 minutes at a flow-rate of 0.40 ml/mln.

Method: 05_B9_{—}1

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C. The UPLC system was connected to two eluent reservoirs containing: A: 0.2 M Na2SO4, 0.04 M H3PO4, 10% CH3CN (pH 3.5); B: 70% CH3CN, 30% H2O. The following linear gradient was used: 70% A, 30% B to 20% A, 80% B over 8 minutes at a flow-rate of 0.40 ml/mln.

Method: 05_B10_{—}1

The RP-analyses was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C. The UPLC system was connected to two eluent reservoirs containing: A: 0.2 M Na2SO4, 0.04 M H3PO4, 10% CH3CN (pH 3.5); B: 70% CH3CN, 30% H2O. The following linear gradient was used: 40% A, 60% B to 20% A, 80% B over 8 minutes at a flow-rate of 0.40 ml/mln.

Method: 07_B4_{—}1

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C.

The UPLC system was connected to two eluent reservoirs containing: A: 99.95% H2O, 0.05% TFA; B: 99.95% CH3CN, 0.05% TFA. The following linear gradient was used: 95% A, 5% B to 5% A, 95% B over 16 minutes at a flow-rate of 0.40 ml/mln.

Method: 09_B2_{—}1

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C. The UPLC system was connected to two eluent reservoirs containing: A: 99.95% H2O, 0.05% TFA; B: 99.95% CH3CN, 0.05% TFA. The following linear gradient was used: 95% A, 5% B to 40% A, 60% B over 16 minutes at a flow-rate of 0.40 ml/mln.

Method: 09_B4_{—}1

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C. The UPLC system was connected to two eluent reservoirs containing: A: 99.95% H2O, 0.05% TFA; B: 99.95% CH3CN, 0.05% TFA. The following linear gradient was used: 95% A, 5% B to 5% A, 95% B over 16 minutes at a flow-rate of 0.40 ml/mln.

Method 08_B2_{—}1

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C.

The UPLC system was connected to two eluent reservoirs containing:

A: 99.95% H2O, 0.05% TFA

B: 99.95% CH3CN, 0.05% TFA

The following linear gradient was used: 95% A, 5% B to 40% A, 60% B over 16 minutes at a flow-rate of 0.40 ml/mln.

Method 08_B4_{—}1

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C.

The UPLC system was connected to two eluent reservoirs containing:

A: 99.95% H2O, 0.05% TFA

B: 99.95% CH3CN, 0.05% TFA

The following linear gradient was used: 95% A, 5% B to 5% A, 95% B over 16 minutes at a flow-rate of 0.40 ml/mln.

Method 10_B4_{—}2

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 50° C.

The UPLC system was connected to two eluent reservoirs containing:

A: 99.95% H2O, 0.05% TFA

B: 99.95% CH3CN, 0.05% TFA

The following linear gradient was used: 95% A, 5% B to 5% A, 95% B over 12 minutes at a flow-rate of 0.40 ml/mln.

Method 10_B5_{—}2

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 50° C.

The UPLC system was connected to two eluent reservoirs containing:

A: 70% MeCN, 30% Water

B: 0.2M Na2SO4, 0.04 M H3PO4, 10% MeCN, pH 2.25

The following linear gradient was used: 40% A in 1 min, 40->70% A in 7 min at a flow-rate of 0.40 ml/mln.

Method: 10_B14_{—}1

The RP-analyses was performed using a Waters UPLC system fitted with a dual band detector. UV detections at 214 nm and 254 nm were collected using an ACQUITY UPLC BEH ShieldRP18, 1.7 um, 2.1 mm×150 mm column, 50° C. The UPLC system was connected to two eluent reservoirs containing: A: 99.95% H2O, 0.05% TFA; B: 99.95% CH3CN, 0.05% TFA. The following linear gradient was used: 70% A, 30% B to 40% A, 60% B over 12 minutes at a flow-rate of 0.40 ml/mln.

Method: AP_B4_{—}1

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 30° C.

The UPLC system was connected to two eluent reservoirs containing: A: 99.95% H2O, 0.05% TFA; B: 99.95% CH3CN, 0.05% TFA. The following linear gradient was used: 95% A, 5% B to 5% A, 95% B over 16 minutes at a flow-rate of 0.30 ml/mln.

### Example 1

### N^{ε24}-[(2S)-2-[[(2S)-2-acetamido-4-carboxybutanoyl]amino]-4-carboxybutanoyl]-[Lys24,Leu27]-Glucagon

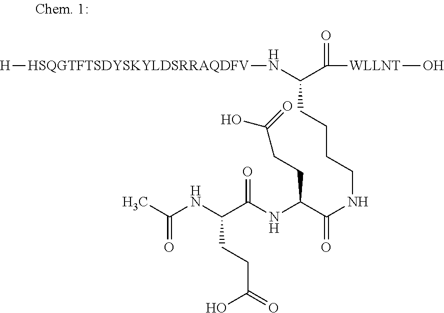

UPLC Method: 08_B2_{—}1: Rt=9.7 min

UPLC Method: 08_B4_{—}1: Rt=6.5 min

UPLC Method: 05_B7_{—}1: Rt=6.0 min

UPLC Method: 04_A9_{—}1: Rt=10.5 min

LCMS Method: LCMS_{—}4: Rt=1.8 min, m/3 1255, m/4=942, m/5=754

### Example 2

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Lys24,Leu27]-Glucagon

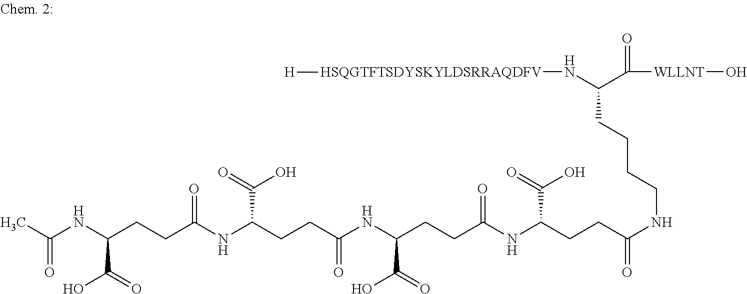

UPLC Method: 09_B2_{—}1: Rt=9.5 min

UPLC Method: 09_B4_{—}1: Rt=6.4 min

UPLC Method: 05_B7_{—}1: Rt=6.4 min

UPLC Method: 04_A9_{—}1: Rt=10.2 min

LCMS Method: LCMS_{—}4: Rt=1.7 min, m/3=1342, m/4=1007

### Example 3

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[His3,Lys24,Leu27,Ser28]-Glucagon

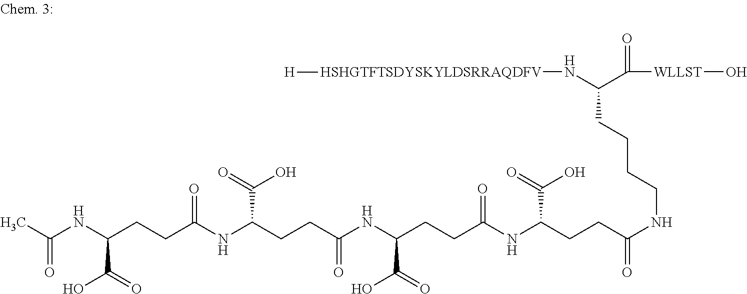

UPLC Method: 09_B2_{—}1: Rt=9.6 min

UPLC Method: 09_B4_{—}1: Rt=6.4 min

UPLC Method: 05_B7_{—}1: Rt=6.6 min

UPLC Method: 04_A9_{—}1: Rt=10.7 min

LCMS Method: LCMS_{—}4: Rt=1.7 min, m/3=1336, m/4=1002

### Example 4

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[His3,Ala20,Lys24,Leu27,Ser28]-Glucagon

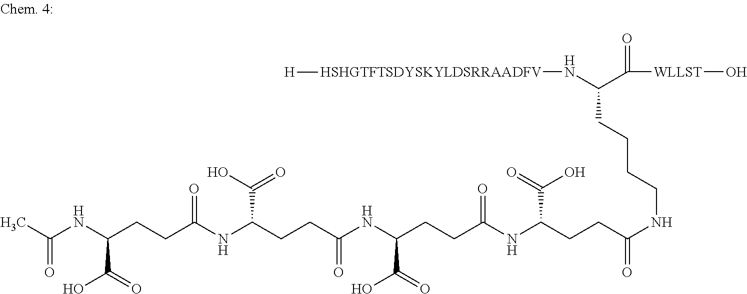

UPLC Method: 09_B2_{—}1: Rt=9.8 min

UPLC Method: 09_B4_{—}1: Rt=6.6 min

UPLC Method: 05_B7_{—}1: Rt=6.8 min

UPLC Method: 04_A9_{—}1: Rt=11.1 min

LCMS Method: LCMS_{—}4: Rt=1.8 min, m/3=1317, m/4=988

### Example 5

### N^{ε24}-[(4S)-4-[[(4S)-4-[[(4S)-4-[[(4S)-4-amino-4-carboxybutanoyl]amino]-4-carboxybutanoyl]amino]-4-carboxybutanoyl]amino]-4-carboxybutanoyl]-[His3,Glu15,Ala20,Lys24,Leu27,Ser28]-Glucagon

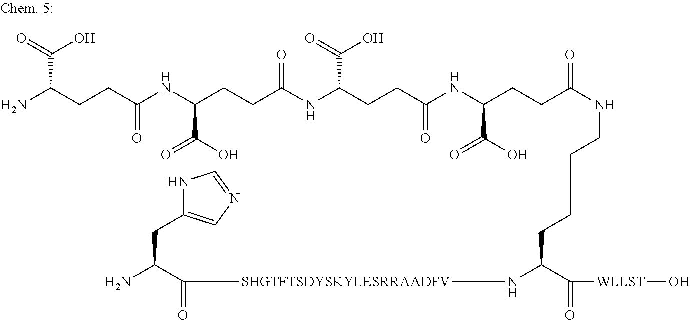

UPLC Method: 09_B4_{—}1: Rt=6.5 min

UPLC Method: 04_A9_{—}1: Rt=8.5 min

LC-MS Method: LCMS_{—}4: RT=2.8; m/3: 1307; m/4: 981; m/5: 785

### Example 6

### N^{ε24}-[(4S)-4-[[(4S)-4-[[(4S)-4-[[(4S)-4-amino-4-carboxybutanoyl]amino]-4-carboxybutanoyl]amino]-4-carboxybutanoyl]amino]-4-carboxybutanoyl]-[His3,Arg12,Glu15,Ala20,Lys24,Leu27,Ser28]-Glucagon

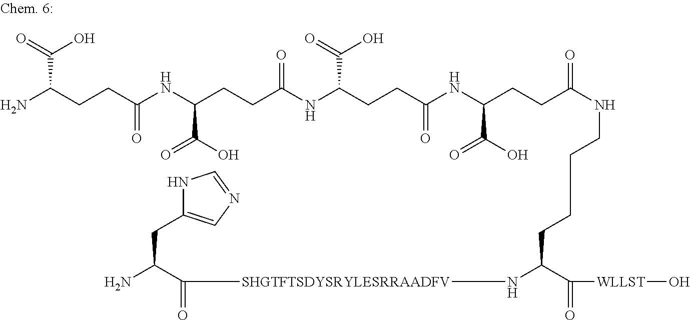

UPLC Method: 09_B4_{—}1: Rt=6.5 min

UPLC Method: 04_A9_{—}1: Rt=8.0 min

LC-MS Method: LCMS_{—}4: RT=2.8; m/3:1317; m/4: 988; m/5: 791

### Example 7

### N^{ε24}-[(4S)-4-[[(4S)-4-[[(4S)-4-[[(4S)-4-amino-4-carboxybutanoyl]amino]-4-carboxybutanoyl]amino]-4-carboxybutanoyl]amino]-4-carboxybutanoyl]-[His3,Thr16,Ala20,Lys24,Leu27,Ser28]-Glucagon

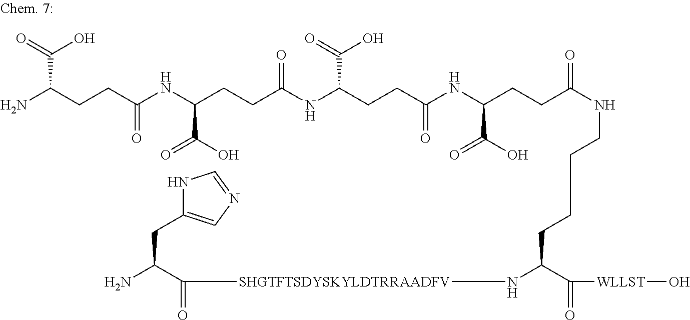

UPLC Method: 09_B4_{—}1: Rt=6.6 min

UPLC Method: 04_A9_{—}1: Rt=10.0 min

LC-MS Method: LCMS_{—}4: Rt=3.6; m/z=3921; m/3:1308; m/4: 981; m/5: 785

### Example 8

### N^{ε24}-[(4S)-4-[[(4S)-4-[[(4S)-4-[[(4S)-4-amino-4-carboxybutanoyl]amino]-4-carboxybutanoyl]amino]-4-carboxybutanoyl]amino]-4-carboxybutanoyl]-[His3,Arg12,Ile16,Ala20,Lys24,Leu27,Ser28]-Glucagon

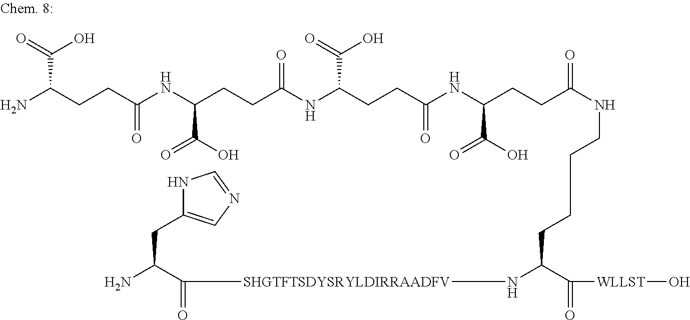

UPLC Method: 09_B4_{—}1: Rt=7.2 min

UPLC Method: 04_A9_{—}1. Rt=13.2 min

LC-MS Method: LCMS_{—}4; RT=3.8; m/3: 1321; m/4: 991; m/5: 793

### Example 9

### N^{ε24}-[(4S)-4-[[(4S)-4-[[(4S)-4-[[(4S)-4-amino-4-carboxybutanoyl]amino]-4-carboxybutanoyl]amino]-4-carboxybutanoyl]amino]-4-carboxybutanoyl]-[His3,Arg12,Thr16,Ala20,Lys24,Leu27,Ser28]-Glucagon

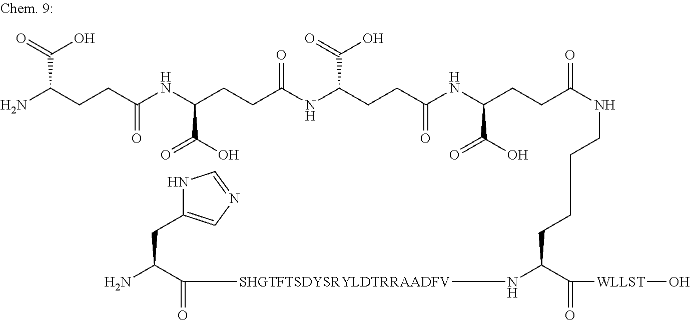

UPLC Method: 09_B4_{—}1: Rt=6.6 min

UPLC Method: 04_A9_{—}1: Rt=11.3 min

LC-MS Method: LCMS_{—}2: Rt=4.6 min, m/3: 1317; m/4: 988

### Example 10

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[His3,Val16,Ala20,Lys24,Leu27,Ser28]-Glucagon

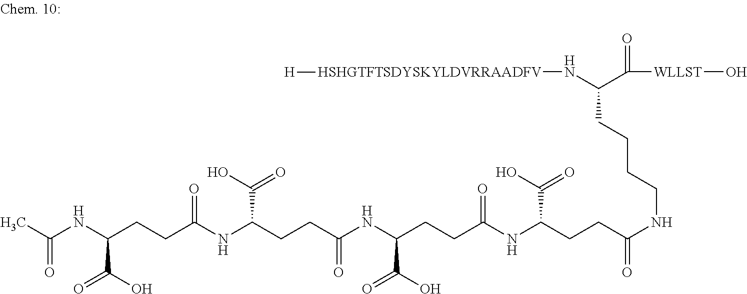

UPLC Method: 09_B2_{—}1: Rt=10.7 min

UPLC Method: 09_B4_{—}1: Rt=7.1 min

UPLC Method: 04_A9_{—}1: Rt=12.7 min

LCMS Method: LCMS_{—}4: Rt=3.1 min, m/3=1321, m/4=991, m/5=793

### Example 11

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[His3,Ile16,Ala20,Lys24,Leu27,Ser28]-Glucagon

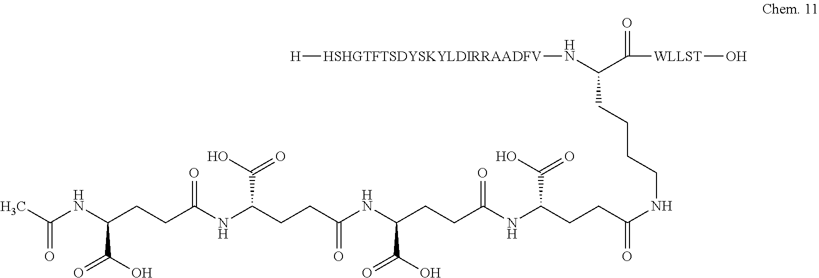

UPLC Method: 09_B2_{—}1: Rt=11.0 min

UPLC Method: 09_B4_{—}1: Rt=7.3 min

UPLC Method: 04_A9_{—}1: Rt=13.2 min

LCMS Method: LCMS_{—}4: Rt=2.0 min, m/3=1326, m/4=994, m/5=796

### Example 12

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[His3,Phe16,Ala20,Lys24,Leu27,Ser28]-Glucagon

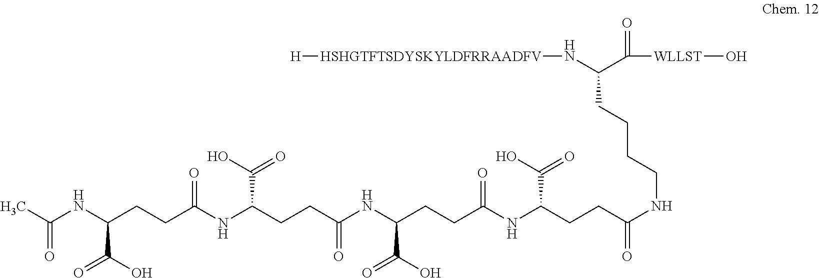

UPLC Method: 09_B2_{—}1: Rt=11.1 min

UPLC Method: 09_B4_{—}1: Rt=7.3 min

UPLC Method: 04_A9_{—}1: Rt=13.1 min

LCMS Method: LCMS_{—}4: Rt=2.0 min, m/3=1337, m/4=1003, m/5=803

### Example 13

### N^{ε24}-[(4S)-4-[[(4S)-4-[[(4S)-4-acetamido-4-carboxybutanoyl]amino]-4-carboxybutanoyl]amino]-4-carboxybutanoyl]-[Lys24,Leu27]-Glucagon

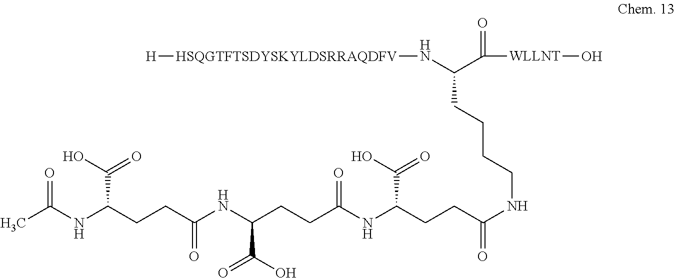

UPLC Method: 09_B2_{—}1: Rt=9.6 min

UPLC Method: 09_B4_{—}1: Rt=6.5 min

UPLC Method: 04_A9_{—}1: Rt=8.1 min

LCMS Method: LCMS_{—}4: Rt=1.8 min, m/3=1299, m/4=974, m/5=780

### Example 14

### N^{ε24}-[(4S)-4-[[(4S)-4-amino-4-carboxybutanoyl]amino]-4-carboxybutanoyl]-[Lys24,Leu27]-Glucagon

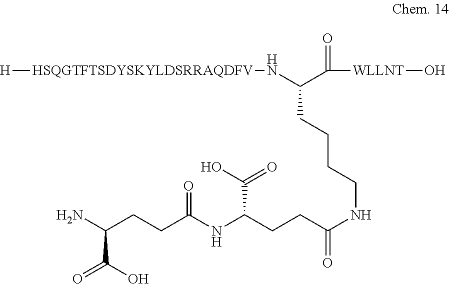

UPLC Method: 09_B2_{—}1: Rt=9.5 min

UPLC Method: 09_B4_{—}1: Rt=6.4 min

UPLC Method: 04_A9_{—}1: Rt=8.5 min

LCMS Method: LCMS_{—}4: Rt=1.7 min, m/3=1242, m/4=932, m/5=745

### Example 15

### N^{ε24}-[(4S)-4-acetamido-4-carboxybutanoyl]-[Lys24,Leu27]-Glucagon

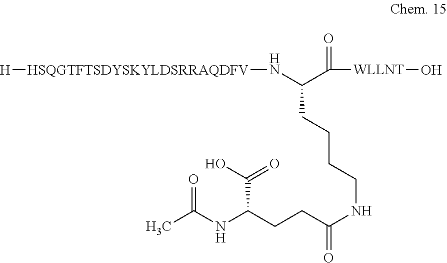

UPLC Method: 09_B2_{—}1: Rt=9.8 min

UPLC Method: 09_B4_{—}1: Rt=6.6 min

UPLC Method: 04_A9_{—}1: Rt=8.7 min

LCMS Method: LCMS_{—}4: Rt=1.7 min, m/3=1213, m/4=910, m/5=728

### Example 16

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Val16,Lys24, Leu27]-Glucagon

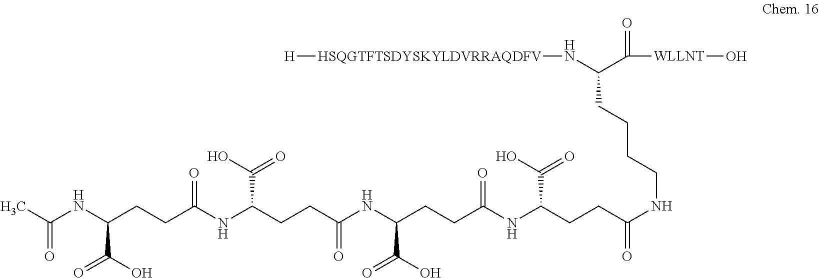

UPLC Method: 09_B2_{—}1: Rt=10.1 min

UPLC Method: 09_B4_{—}1: Rt=6.7 min

UPLC Method: 04_A9_{—}1: Rt=11.5 min

LCMS Method: LCMS_{—}4: Rt=1.9 min, m/3=1346; m/4=1010; m/5=808

### Example 17

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Glu15,Lys24,Leu27]-Glucagon

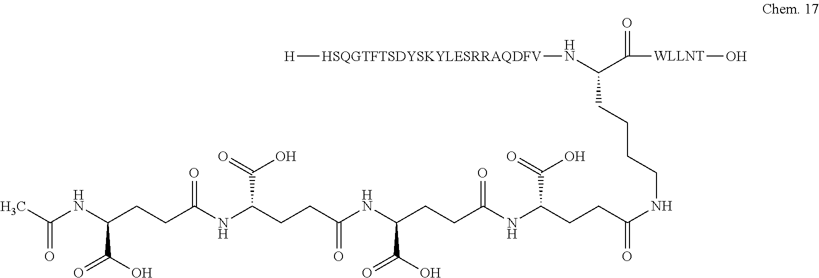

UPLC Method: 09_B2_{—}1: Rt=9.5 min

UPLC Method: 09_B4_{—}1: Rt=6.4 min

UPLC Method: 04_A9_{—}1: Rt=8.2 min

LCMS Method: LCMS_{—}4: Rt=1.7 min, m/3=1347; m/4=1010; m/5=809

### Example 18

### N^{ε24}-[(2S)-2-[[(2S)-2-[[(2S)-2-acetamido-4-carboxybutanoyl]amino]-4-carboxybutanoyl]amino]-4-carboxybutanoyl]-[Lys24,Leu27]-Glucagon

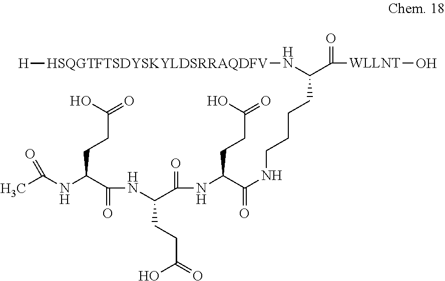

UPLC Method: 09_B2_{—}1: Rt=9.7 min

UPLC Method: 09_B4_{—}1: Rt=6.5 min

UPLC Method: 04_A9_{—}1: Rt=9.1 min

LCMS Method: LCMS_{—}4: Rt=1.7 min, m/3=1299; m/4=974; m/5=780

### Example 19

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[His3,Lys24,Leu27]-Glucagon

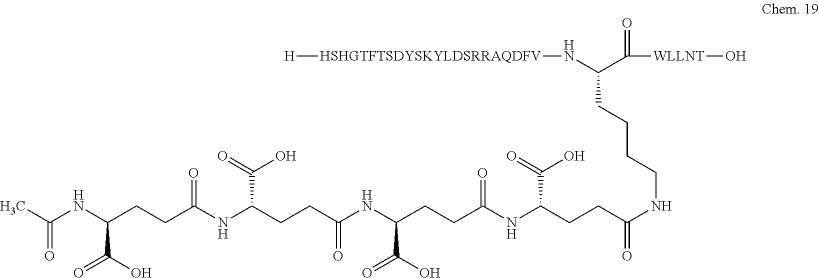

UPLC Method: 09_B2_{—}1: Rt=9.4 min

UPLC Method: 09_B4_{—}1: Rt=6.3 min

UPLC Method: 04_A9_{—}1: Rt=9.4 min

LCMS Method: LCMS_{—}4: Rt=1.7 min, m/3=1345; m/4=1009; m/5=807

### Example 20

### N^{ε24}-[(2S)-2-[[(2S)-2-[[(2S)-2-amino-4-carboxybutanoyl]amino]-4-carboxybutanoyl]amino]-4-carboxybutanoyl]-[Lys24,Leu27]-Glucagon

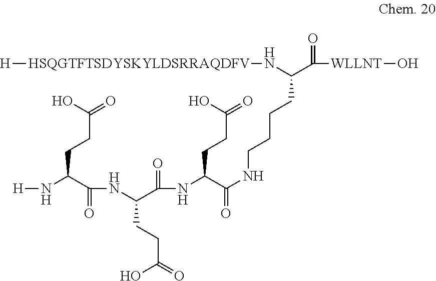

UPLC Method: 09_B2_{—}1: Rt=9.3 min

UPLC Method: 09_B4_{—}1: Rt=6.3 min

UPLC Method: 04_A9_{—}1: Rt=9.2 min

LCMS Method: LCMS_{—}4: Rt=1.7 min, m/3=1285; m/4=964; m/5=771

### Example 21

### N^{ε24}-[(2S)-2-[[(2S)-2-[[(2S)-2-[[(2S)-2-amino-4-carboxybutanoyl]amino]-4-carboxybutanoyl]amino]-4-carboxybutanoyl]amino]-4-carboxybutanoyl]-[Lys24, Leu27]-Glucagon

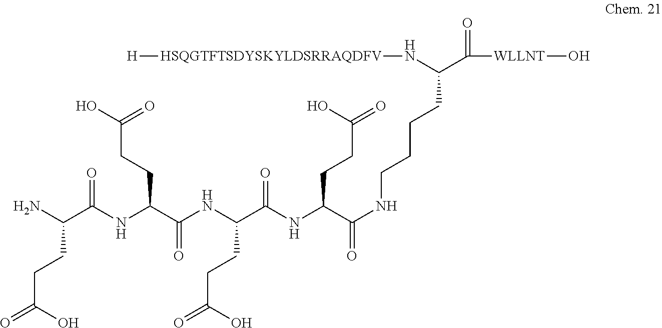

UPLC Method: 09_B2_{—}1: Rt=9.3 min

UPLC Method: 09_B4_{—}1: Rt=6.3 min

UPLC Method: 04_A9_{—}1: Rt=9.4 min

LCMS Method: LCMS_{—}4: Rt=1.7 min, m/3=1328; m/4=996; m/5=797

### Example 22

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Glu15,Glu21,Lys24,Leu27,Ser28]-Glucagon

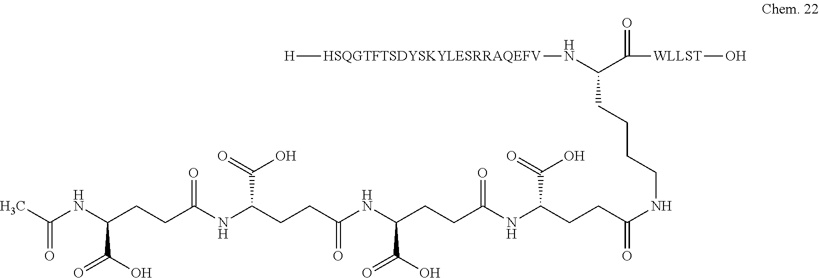

UPLC Method: 09_B2_{—}1: Rt=9.8 min

UPLC Method: 09_B4_{—}1: Rt=6.6 min

UPLC Method: 04_A9_{—}1: Rt=9.0 min

LCMS Method: LCMS_{—}4: Rt=1.8 min, m/3=1343; m/4=1007; m/5=806

### Example 23

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Glu15,Lys24,Leu27,Ser28]-Glucagon

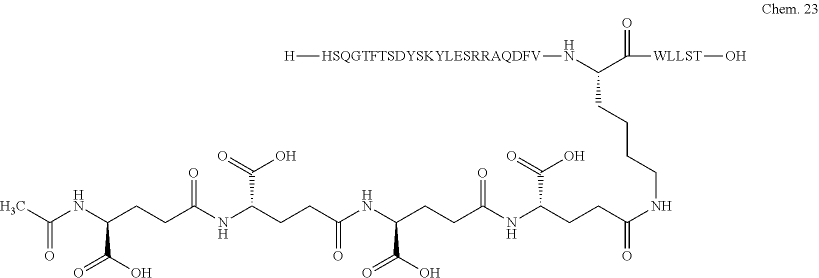

UPLC Method: 09_B2_{—}1: Rt=9.7 min

UPLC Method: 09_B4_{—}1: Rt=6.5 min

UPLC Method: 04_A9_{—}1: Rt=8.8 min

LCMS Method: LCMS_{—}4: Rt=1.8 min, m/3=1338; m/4=1003; m/5=803

### Example 24

### N^{ε24}-[(4S)-4-[[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]amino]-4-carboxybutanoyl]-[Lys24,Leu27]-Glucagon

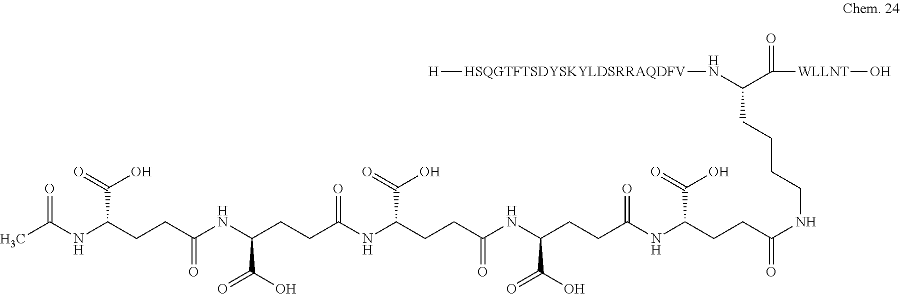

UPLC Method: 09_B2_{—}1: Rt=9.5 min

UPLC Method: 09_B4_{—}1: Rt=6.4 min

UPLC Method: 04_A9_{—}1: Rt=9.1 min

LCMS Method: LCMS_{—}4: Rt=1.8 min, m/3=1385; m/4=1039; m/5=832

### Example 25

### N^{ε24}-[(4S)-4-[[(4S)-4-[[(4S)-4-[[(4S)-4-[[-4-[[(4S)-4-[[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]amino]-4-carboxybutanoyl]amino]-4-carboxybutanoyl]amino]-4-carboxybutanoyl]amino]-4-carboxybutanoyl]amino]-4-carboxybutanoyl]amino]-4-carboxybutanoyl]-[Lys24,Leu27]-Glucagon

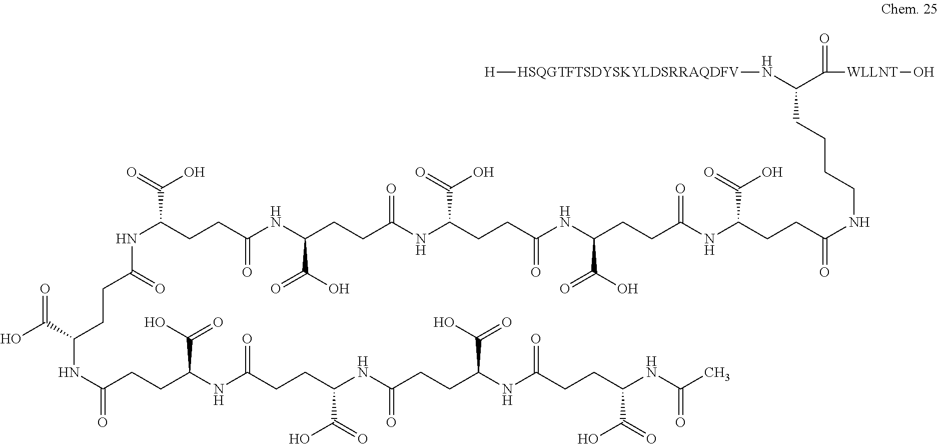

UPLC Method: 09_B2_{—}1: Rt=9.4 min

UPLC Method: 09_B4_{—}1: Rt=6.3 min

UPLC Method: 04_A9_{—}1: Rt=8.7 min

LCMS Method: LCMS_{—}4: Rt=1.8 min, m/3=1601; m/4=1201; m/5=961

### Example 26

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Arg12,Thr16,Lys24,Leu27,Ser28]-Glucagonyl-Pro

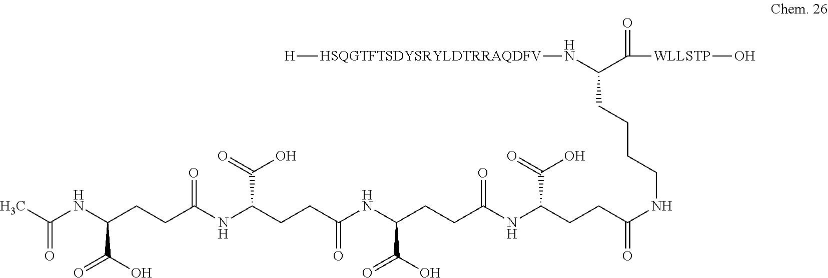

UPLC Method: 04_A9_{—}1: Rt=10.9 min

UPLC Method: 09_B2_{—}1: Rt=9.9 min

LCMS Method: LCMS_{—}4: Rt=1.8 min, m/3=1380; m/4=1035; m/5=828

### Example 27

### N^{ε12}-[(2S)-6-[[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]amino]-2-aminohexanoyl]-[Leu27]-Glucagon

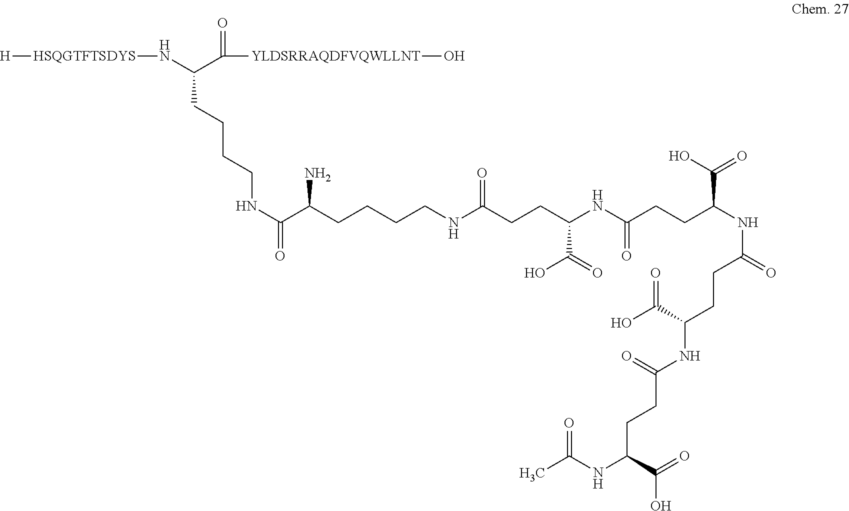

UPLC Method: 09_B2_{—}1: Rt=9.7 min

UPLC Method: 09_B4_{—}1: Rt=6.6 min

UPLC Method: 04_A9_{—}1: Rt=9.8 min

LCMS Method: LCMS_{—}4: Rt=1.8 min, m/3=1385; m/4=1039; m/5=831

### Example 28

### N^{ε12}-[2-[[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]amino]acetyl]-[Leu27]-Glucagon

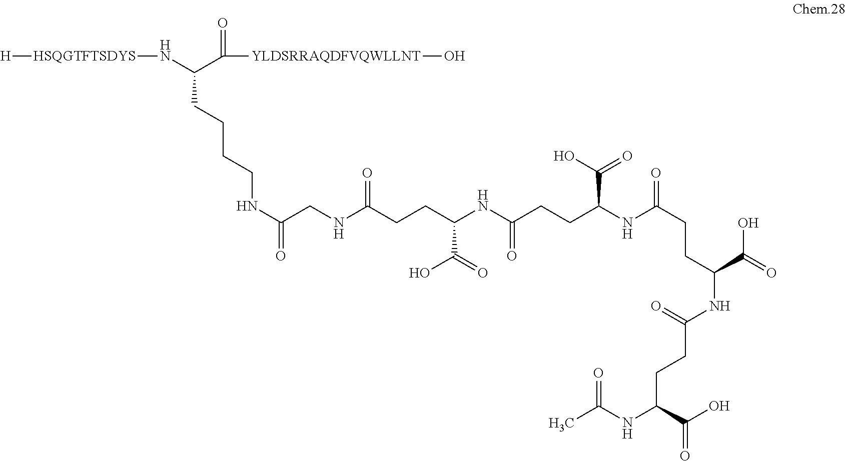

UPLC Method: 09_B2_{—}1: Rt=6.8 min

UPLC Method: 09_B4_{—}1: Rt=10.0 min

UPLC Method: 04_A9_{—}1: Rt=9.6 min

LCMS Method: LCMS_{—}4: Rt=1.9 min, m/3=1361; m/4=1021

### Example 29

### N^{ε12}-[(2S)-2-[[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]amino]-5-carbamimidamidopentanoyl]-[Leu27]-Glucagon

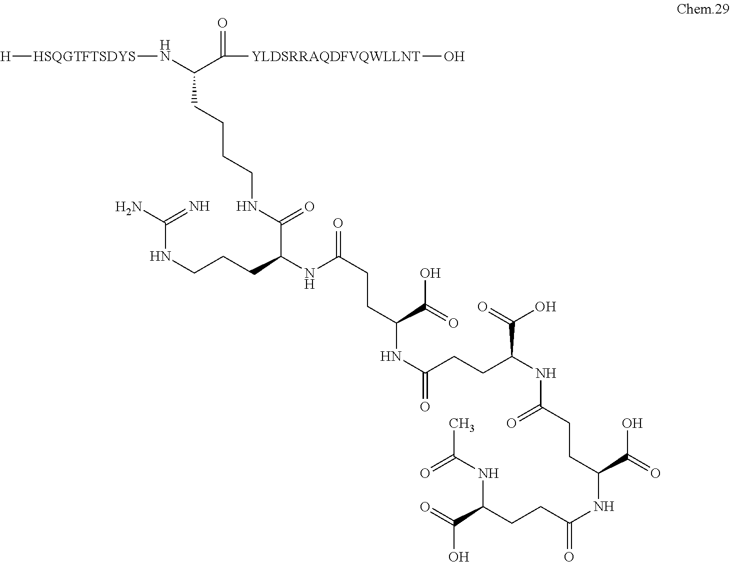

UPLC Method: 09_B2_{—}1: Rt=9.7 min

UPLC Method: 09_B4_{—}1: Rt=6.6 min

UPLC Method: 04_A9_{—}1: Rt=9.6 min

LCMS Method: LCMS_{—}4: Rt=1.8 min, m/3=1394; m/4=1046; m/5=837

### Example 30

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Thr16,Lys24,Leu27,Ser28]-Glucagon

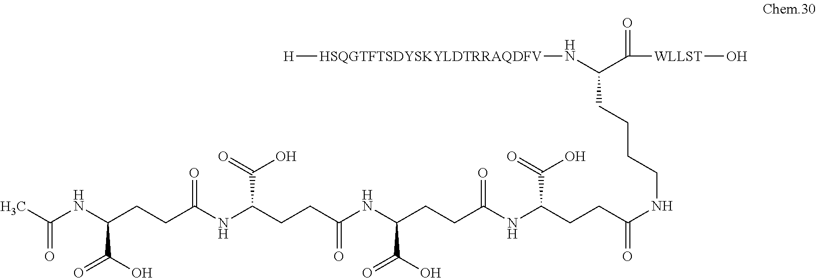

UPLC Method: 09_B2_{—}1: Rt=9.9 min

UPLC Method: 09_B4_{—}1: Rt=6.6 min

UPLC Method: 04_A9_{—}1: Rt=10.6 min

LCMS Method: LCMS_{—}4: Rt=1.9 min, m/3=1338; m/4=1003; m/5=803

### Example 31

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[His3,Glu15,Thr16,Lys24,Leu27,Ser28]-Glucagon

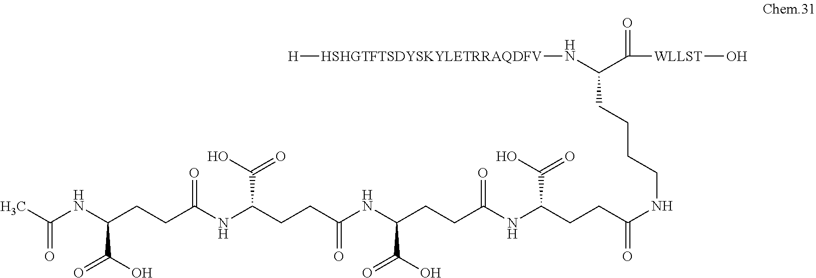

UPLC Method: 09_B2_{—}1: Rt=9.7 min

UPLC Method: 09_B4_{—}1: Rt=6.5 min

UPLC Method: 04_A9_{—}1: Rt=9.6 min

LCMS Method: LCMS_{—}4: Rt=1.9 min, m/3=1346; m/4=1010; m/5=808

### Example 32

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[His3,Thr16,Lys24,Leu27,Ser28]-Glucagon

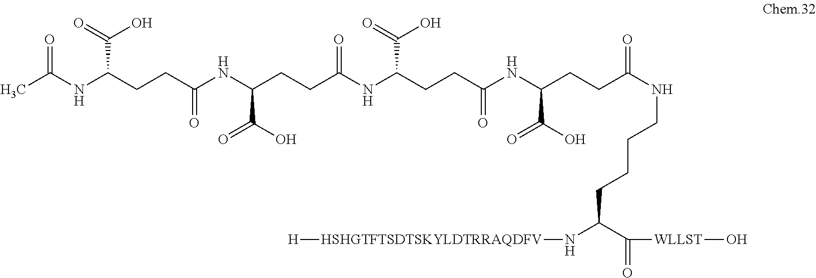

UPLC Method: 09_B2_{—}1: Rt=9.7 min

UPLC Method: 09_B4_{—}1: Rt=6.5 min

UPLC Method: 04_A9_{—}1: Rt=10.8 min

LCMS Method: LCMS_{—}4: Rt=1.9 min, m/3=1341; m/4=1006; m/5=805

### Example 33

### N^{ε24}-[(4S)-4-[[(4S)-4-[[(4S)-4-[[(4S)-4-amino-4-carboxybutanoyl]amino]-4-carboxybutanoyl]amino]-4-carboxybutanoyl]amino]-4-carboxybutanoyl]-[His3,Thr16,Lys24,Leu27,Ser28]-Glucagon

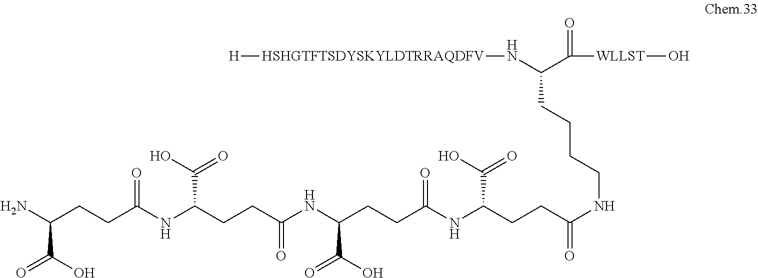

UPLC Method: 09_B2_{—}1: Rt=9.5 min

UPLC Method: 09_B4_{—}1: Rt=6.4 min

UPLC Method: 04_A9_{—}1: Rt=10.8 min

LCMS Method: LCMS_{—}4: Rt=1.9 min, m/3=1327; m/4=995; m/5=796

### Example 34

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Trp16,Lys24,Leu27,Ser28]-Glucagon

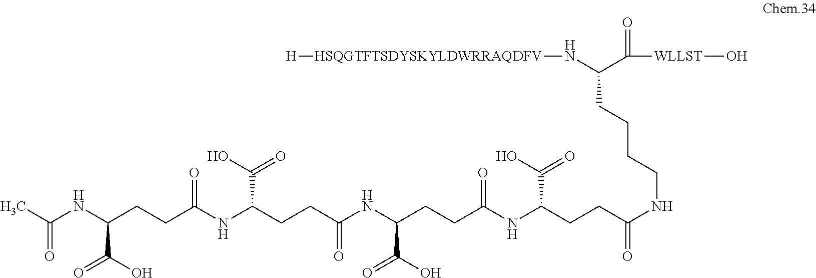

UPLC Method: 04_A9_{—}1: Rt=13.3 min

UPLC Method: 09_B2_{—}1: Rt=10.6 min

LCMS Method: LCMS_{—}4: Rt=2.1 min, m/3=1366; m/4=1025; m/5=820

### Example 35

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Phe16,Lys24,Leu27,Ser28]-Glucagon

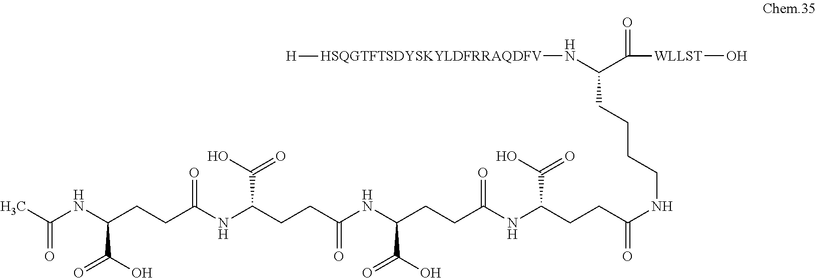

UPLC Method: 04_A9_{—}1: Rt=13.1 min

UPLC Method: 09_B2_{—}1: Rt=10.6 min

LCMS Method: LCMS_{—}4: Rt=2.1 min, m/3=1353; m/4=1015; m/5=812

### Example 36

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Ile16,Lys24,Leu27,Ser28]-Glucagon

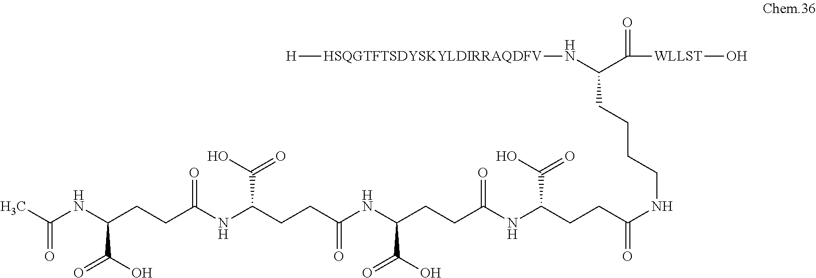

UPLC Method: 04_A9_{—}1: Rt=13.1 min

UPLC Method: 09_B2_{—}1: Rt=10.6 min

LCMS Method: LCMS_{—}4: Rt=2.1 min, m/3=1342; m/4=1006; m/5=806

### Example 37

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Tyr16,Lys24,Leu27,Ser28]-Glucagon

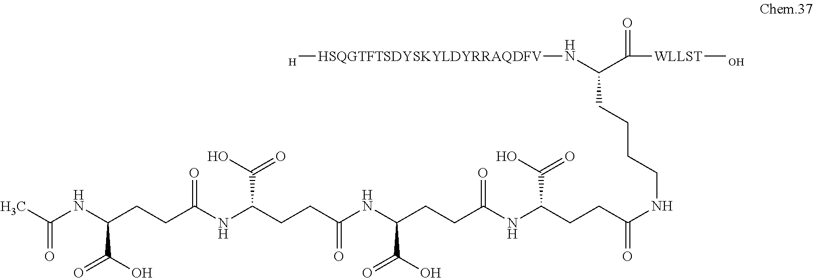

UPLC Method: 04_A9_{—}1: Rt=11.5 min

UPLC Method: 09_B2_{—}1: Rt=10.1 min

LCMS Method: LCMS_{—}4: Rt=2.0 min, m/3=1359; m/4=1019; m/5=816

### Example 38

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Leu16,Lys24,Leu27,Ser28]-Glucagon

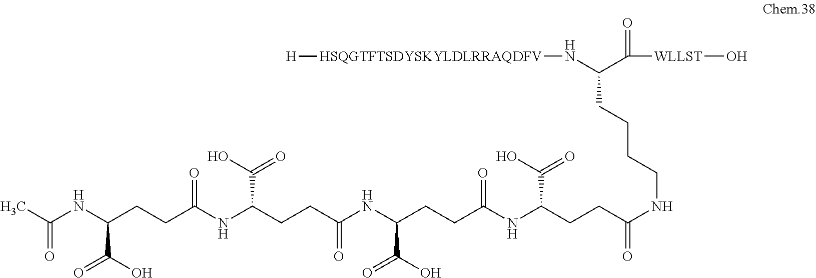

UPLC Method: 04_A9_{—}1: Rt=14.1 min

UPLC Method: 09_B4_{—}1: Rt=7.1 min

LCMS Method: LCMS_{—}4: Rt=2.1 min, m/3=1342; m/4=1007; m/5=806

### Example 39

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Arg12,Leu16,Lys24,Leu27,Ser28]-Glucagon

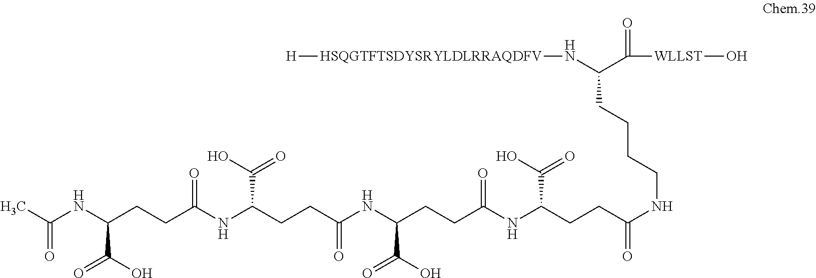

UPLC Method: 04_A9_{—}1: Rt=14.1 min

UPLC Method: 09_B2_{—}1: Rt=10.7 min

LCMS Method: LCMS_{—}4: Rt=2.1 min, m/3=1350; m/4=1013; m/5=811

### Example 40

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Arg12,Leu16,Lys24,Leu27,Ser28]-Glucagonyl-Pro

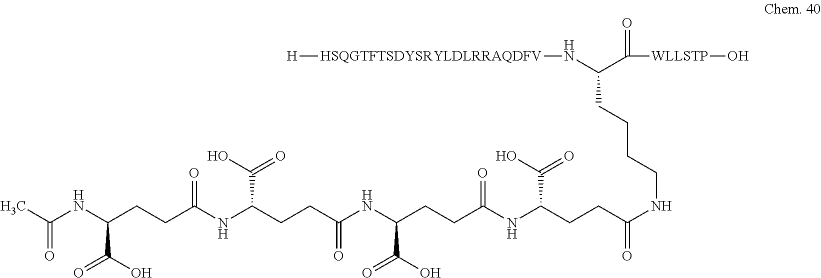

UPLC Method: 04_A9_{—}1: Rt=14.0 min

UPLC Method: 09_B2_{—}1: Rt=10.7 min

LCMS Method: LCMS_{—}4: Rt=2.1 min, m/3=1384; m/4=1038; m/5=831

### Example 41

### N^{e24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Thr²,Leu¹⁶,Lys²⁴,Leu²⁷,Ser²⁸]-Glucagon

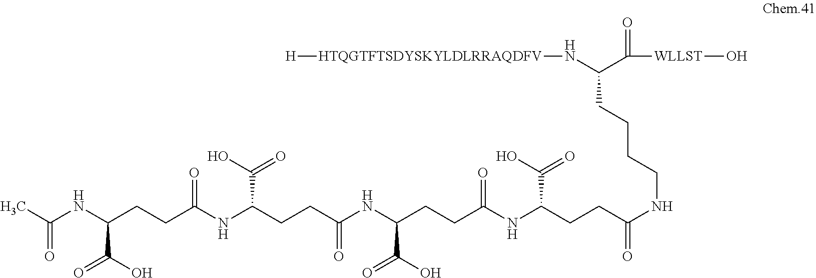

UPLC Method: 08_B2_{—}1: Rt=10.4 min

UPLC Method: 04_A9_{—}1: Rt=12.8 min

LCMS Method: LCMS_{—}4: Rt=2.1 min, m/3=1347; m/4=1010; m/5=808

### Example 42

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Ala²,Leu¹⁶,Lys²⁴,Leu²⁷,Ser²⁸]-Glucagon

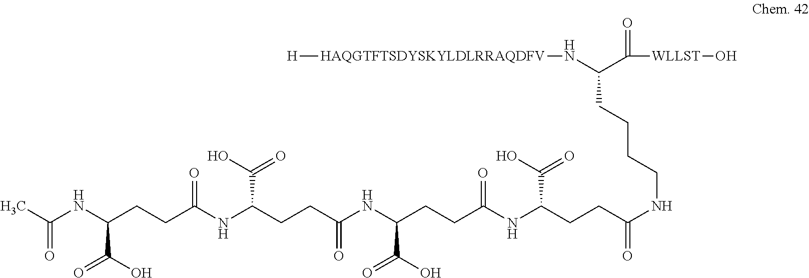

UPLC Method: 08_B2_{—}1: Rt=10.4 min

UPLC Method: 04_A9_{—}1: Rt=12.8 min

LCMS Method: LCMS_{—}4: Rt=2.0 min, m/3=1337; m/4=1002; m/5=802

### Example 43

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Val¹⁰,Leu¹⁶,Lys²⁴,Leu²⁷,Ser²⁸]-Glucagon

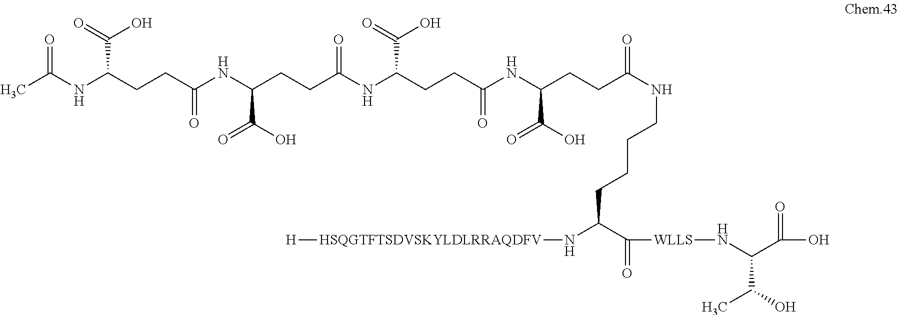

UPLC Method: UPLC_AP: Rt=7.16 min

LCMS Method: LCMS_AP: Rt=4.9 min, m/2=1980; m/3=1320

### Example 44

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Val¹⁰,Glu¹⁵,Lys²⁴,Leu²⁷,Ser²⁸]-Glucagon

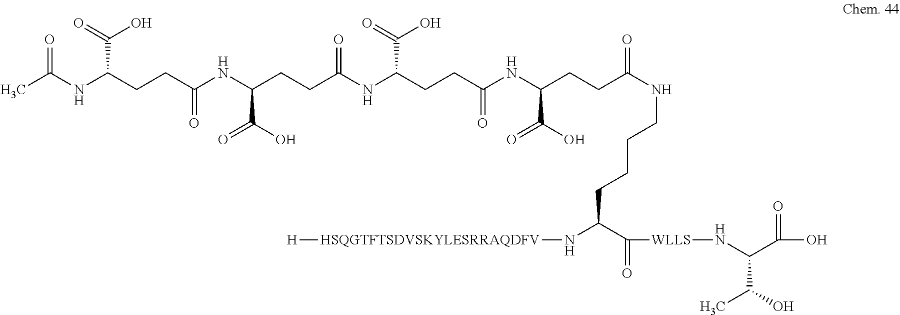

UPLC Method: UPLC_AP: Rt=6.53 min

LCMS Method: LCMS_AP: Rt=4.8 min, m/2=1973; m/3=1316

### Example 45

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Val¹⁰,Glu¹⁵,Glu²¹,Lys²⁴,Leu²⁷,Ser²⁸]-Glucagon

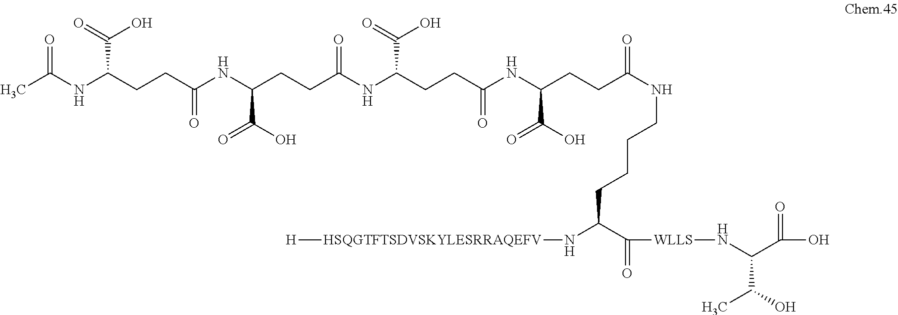

UPLC Method: UPLC_AP: Rt=6.57 min

LCMS Method: LCMS_AP: Rt=4.7 min, m/2=1980; m/3=1320

### Example 46

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Gly²,His³,Val¹⁶,Ala²⁰,Glu²¹,Lys²⁴,Leu²⁷,Ser²⁸]-Glucagon

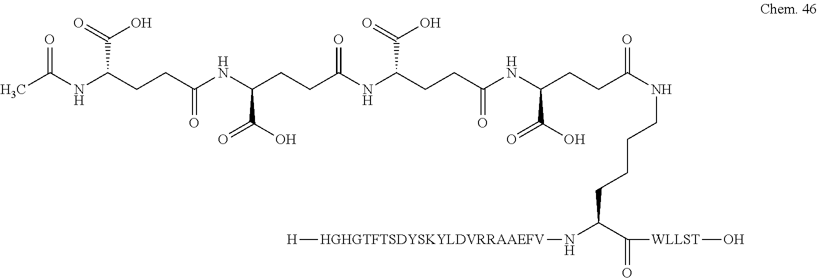

UPLC Method: 05_B4_{—}1: Rt=7.0 min

LCMS Method: LCMS_{—}4: Rt=2.0 min, m/3=1315; m/4=987; m/5=790

UPLC Method: 01_A9_{—}1: Rt=16.5 min

### Example 47

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Ala²,His³,Val¹⁶,Ala²⁰,Glu²¹,Lys²⁴,Leu²⁷,Ser²⁸]-Glucagon

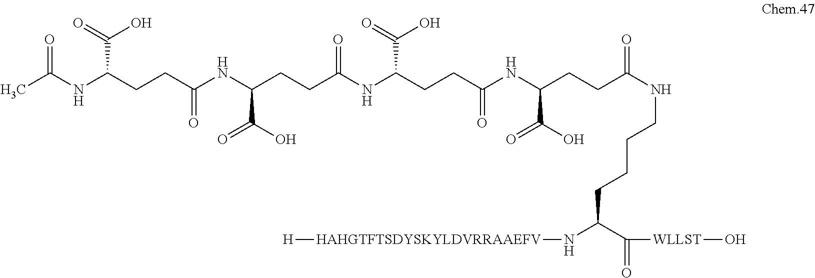

UPLC Method: 05_B4_{—}1: Rt=7.0 min

UPLC Method: 01_A9_{—}1: Rt=16.6 min

LCMS Method: LCMS_{—}4: Rt=2.0 min, m/3=1320; m/4=990; m/5=792

### Example 48

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Aib²,His³,Val¹⁶,Ala²⁰,Glu²¹,Lys²⁴,Leu²⁷,Ser²⁸]-Glucagon

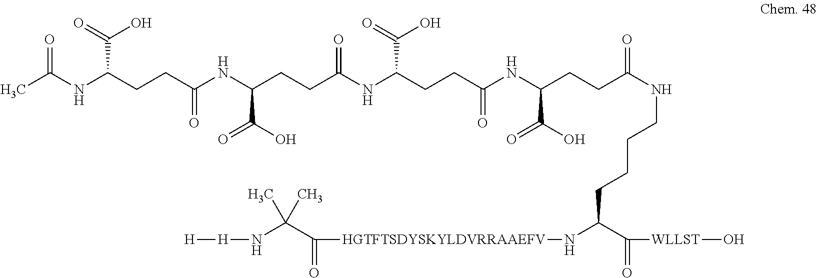

UPLC Method: 05_B4_{—}1: Rt=7.0 min

UPLC Method: 01_A9_{—}1: Rt=16.8 min

LCMS Method: LCMS_{—}4: Rt=2.0 min, m/3=1325; m/4=994; m/5=795

### Example 49

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Gly²,Val¹⁶,Ala²⁰,Glu²¹,Lys²⁴,Leu²⁷,Ser²⁸]-Glucagon

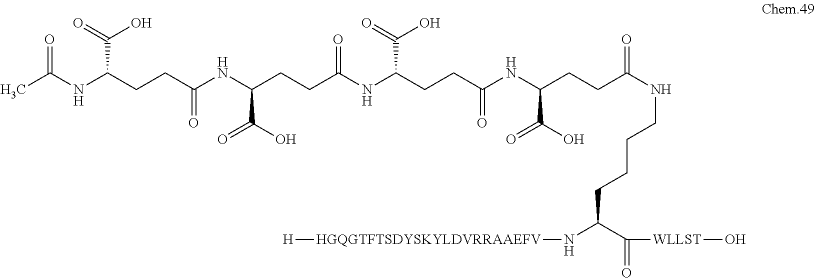

UPLC Method: 05_B4_{—}1: Rt=7.0 min

UPLC Method: 01_A9_{—}1: Rt=16.4 min

LCMS Method: LCMS_{—}4: Rt=2.0 min, m/3=1313; m/4=985; m/5=788

### Example 50

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Gly²,His³,Val¹⁰,Val¹⁶,Ala²⁰,Glu²¹,Lys²⁴,Leu²⁷,Ser²⁸]-Glucagon

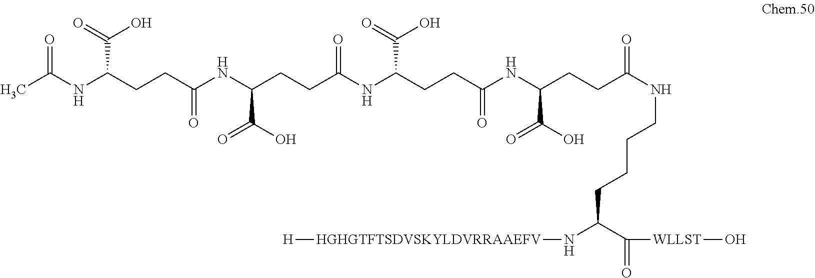

UPLC Method: 05_B4_{—}1: Rt=7.1 min

UPLC Method: 01_A9_{—}1: Rt=17.2 min

LCMS Method: LCMS_{—}4: Rt=2.1 min, m/3=1294; m/4=971; m/5=777

### Example 51

### N^{α}([Leu²⁷,Ser²⁸]-Glucagonyl)-N^{ε}[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]Lys

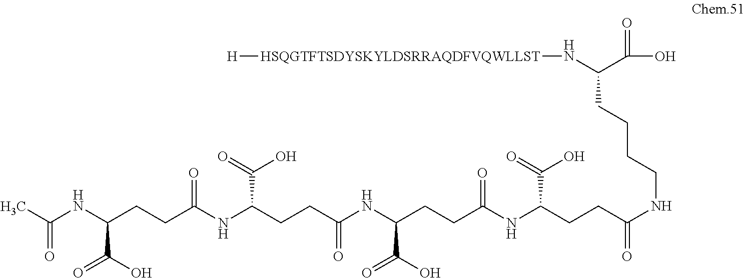

UPLC Method: LCMS_{—}4 UPLC02v01: Rt=6.22 min;

LCMS Method: LCMS_{—}4: Rt=1.80 min; calc. m/z; 4124.44; m/3 1375.81; m/4 1032.11; m/5 825.88. found m/z 4124.2; m/3 1375.6; m/4 1031.7; m/5 825.8

### Example 52

### N^{ε29}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Leu²⁷,Ser²⁸,Lys²⁹]-Glucagon

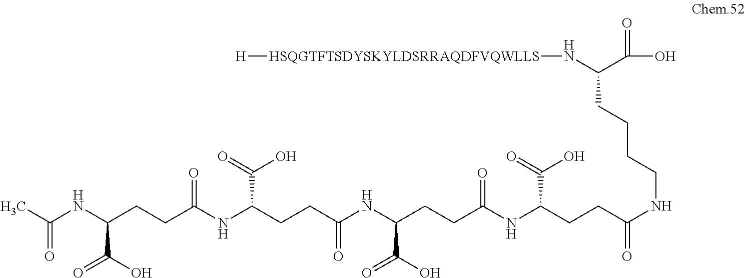

UPLC Method: 09_B2_{—}1: Rt=9.35 min

LCMS Method: LCMS_{—}4 Rt=1.75 min. calc. m/z 4023.33; m/3 1342.11; m/4 1006.83; m/5 805.66. found m/z 4022.6; m/3 1341.9; m/4 1006.4; m/5 805.6.

### Example 53

### N^{ε28}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Leu²⁷,Lys²⁸]-Glucagon

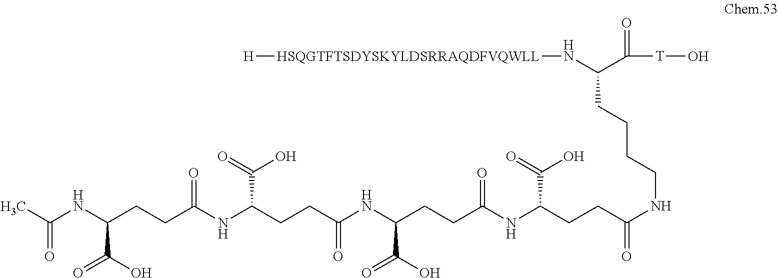

UPLC Method: 09_B2_{—}1: Rt=9.26 min

LCMS Method: LCMS_{—}4; Rt=1.77 min calc. m/z 4037.36; m/3 1346.78; m/4 1010.34; m/5 808.47. found m/z 4036.9; m/3 1346.6; m/4 1010.2; m/5 808.4.

### Example 54

### N^{ε20}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Lys²⁰,Leu²⁷,Ser²⁸]-Glucagon

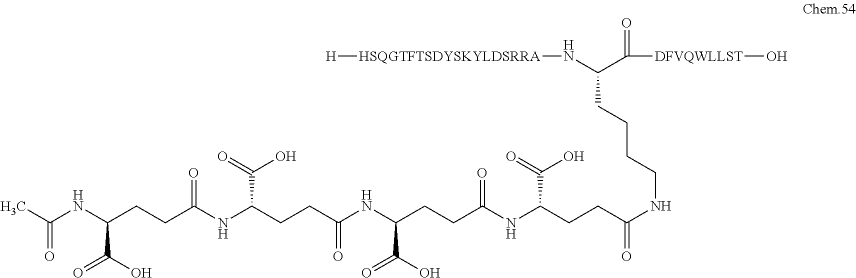

UPLC Method: 09_B2_{—}1: Rt=9.76 min

LCMS Method: LCMS_{—}4: Rt=1.85 min calc. m/z 3996.31; m/3 1333.10; m/4 1000.07; m/5 800.26. found m/z 3995.6; m/3 1332.9; m/4 999.7; m/5 800.2.

### Example 55

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Gly²,Leu¹⁶,Lys²⁴,Leu²⁷,Ser²⁸]-Glucagon

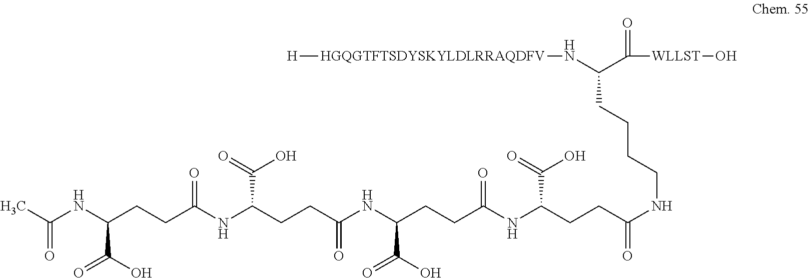

UPLC Method: 09_B2_{—}1: Rt=10.49 min

LCMS Method: LCMS_{—}4: Rt=2.01 min. calc. m/z 3992.36; m/3 1331.78; m/4 999.09; m/5 799.47. found m/z 3992.2; m/3 1331.6; m/4 998.7; m/5 799.4.

### Example 56

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Arg¹²,Leu¹⁶,Ala²⁰,Lys²⁴,Leu²⁷,Ser²⁸]-Glucagon

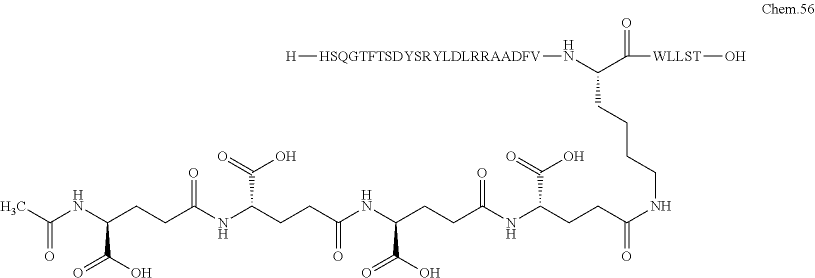

UPLC Method: 09_B2_{—}1: Rt=11.05 min

LCMS Method: LCMS_{—}4: Rt=2.10 min. calc. m/z 3993.35; m/3 1332.11; m/4 999.33; m/5 799.67. found m/z 3993.1; m/3 1332.0; m/4 998.9; m/5 799.6.

### Example 57

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Arg¹²,Leu¹⁶,Glu²⁰,Lys²⁴,Leu²⁷,Ser²⁸]-Glucagon

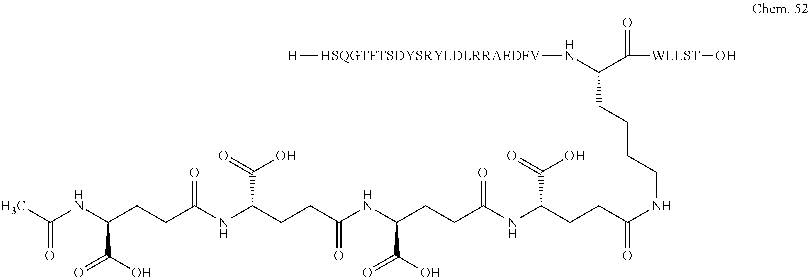

UPLC Method: 09_B2_{—}1: Rt=10.93 min;

LCMS Method: LCMS_{—}4: calc. m/z 4051.39; m/3 1351.46; m/4 1013.84; m/5 811.27. found m/z 4050.7; m/3 1351.3; m/4 1013.4; m/5 811.2.

### Example 58

### N^{ε24}-[(2S)-6-[[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]amino]-2-aminohexanoyl]-[Lys²⁴,Leu²⁷]-Glucagon

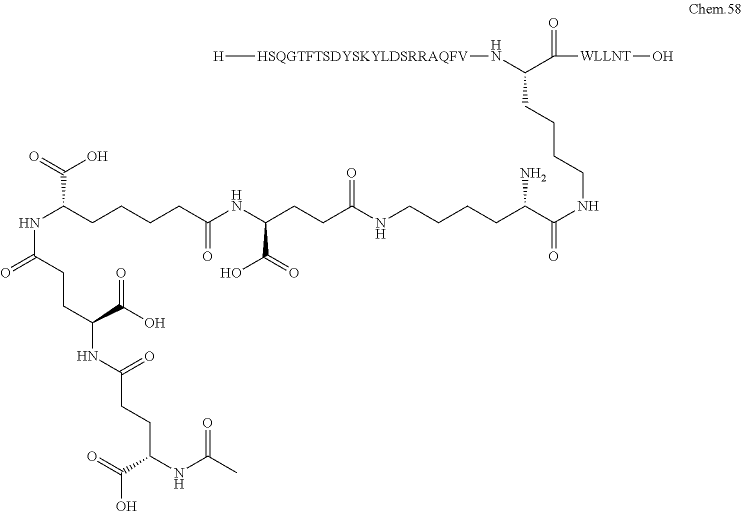

UPLC Method: 09_B2_{—}1: Rt=8.9 min

UPLC Method: 04_A9_{—}1: Rt=9.4 min

LCMS Method: LCMS_{—}4: Rt=1.9 min, m/4=1038.7; m/5=831.1; m/5=692.9

### Example 59

### N^{ε16}-[(2S)-6-[[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]amino]-2-aminohexanoyl]-[Lys¹⁶,Ala¹⁸,Leu²⁷,Ser²⁸]-Glucagon amide

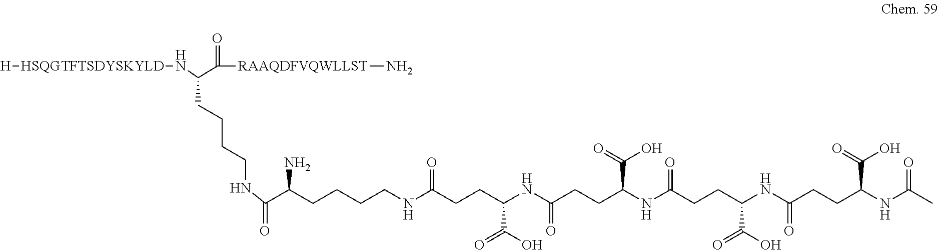

UPLC Method: 09_B2_{—}1: Rt=9.8 min

UPLC Method: 04_A9_{—}1: Rt=11.7 min

LCMS Method: LCMS_{—}4: Rt=1.9 min, m/3=1360; m/4=1020; m/5=816

### Example 60

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Ala²,His³,Leu¹⁶,Lys²⁴,Leu²⁷,Ser²⁸]-Glucagon

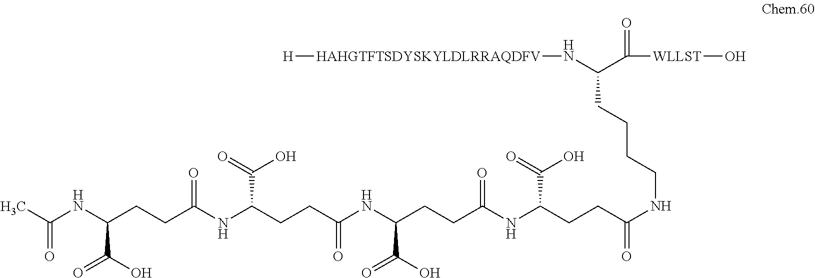

UPLC Method: UPLC_AP Rt=6.79 min

### Example 61

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Ala²,Val¹⁰,Leu¹⁶,Ala²⁰,Lys²⁴,Leu²⁷,Ser²⁸]-Glucagon

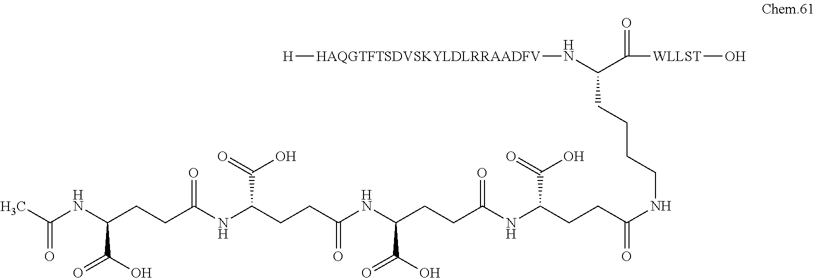

UPLC Method: UPLC_AP Rt=7.38 min;

### Example 62

### N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Ala²,His³,Val¹⁰,Leu¹⁶,Ala²⁰,Lys²⁴,Leu²⁷,Ser²⁸]-Glucagon

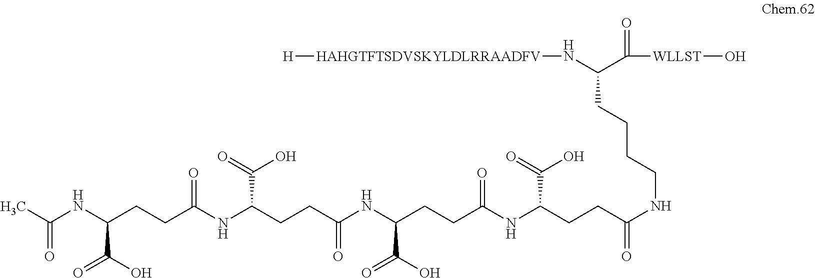

UPLC Method: UPLC_AP Rt=7.19 min

### Example 63

### N^{ε24}-[(4S)-4-carboxy-4-[[(4S)-4-carboxy-4-[[(4S)-4-carboxy-4-[[(4S)-4-carboxy-4-(propanoylamino)butanoyl]amino]butanoyl]amino]butanoyl]amino]butanoyl]-[Arg¹²,Leu¹⁶,Ala²⁰,Lys²⁴,Leu²⁷,Ser²⁸]-Glucagon

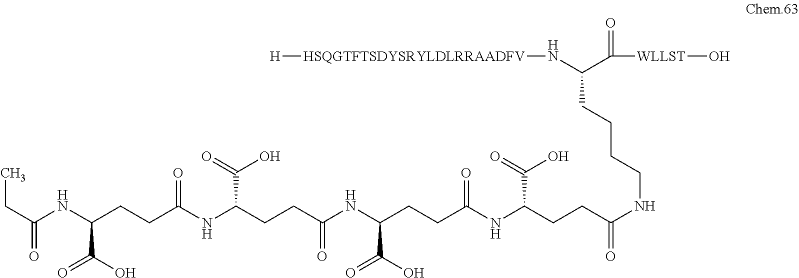

UPLC Method: UPLC_AP Rt=7.24 min

### Example 64

### N^{ε24}-[(4S)-4-carboxy-4-[[(4S)-4-carboxy-4-[[(4S)-4-carboxy-4-[[(4S)-4-carboxy-4-(2-methylpropanoylamino)butanoyl]amino]butanoyl]amino]butanoyl]amino]butanoyl]-[Arg¹²,Leu¹⁶,Ala²⁰,Lys²⁴,Leu²⁷,Ser²⁸]-Glucagon

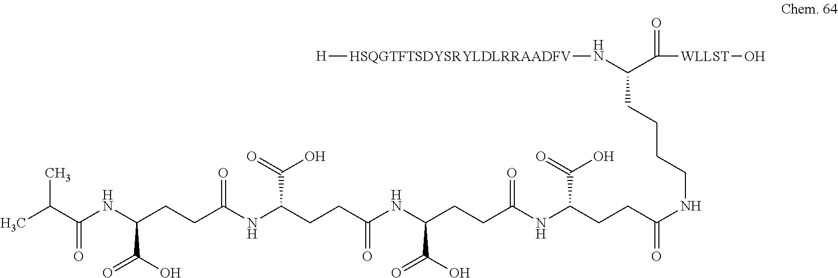

UPLC Method: UPLC_AP Rt=7.31 min

### Example 65

### N^{ε24}-[(4S)-4-[[(4S)-4-[[(4S)-4-[[(4S)-4-(butanoylamino)-4-carboxybutanoyl]amino]-4-carboxybutanoyl]amino]-4-carboxybutanoyl]amino]-4-carboxybutanoyl]-[Arg¹²,Leu¹⁶,Ala²⁰,Lys²⁴,Leu²⁷,Ser²⁸]-Glucagon

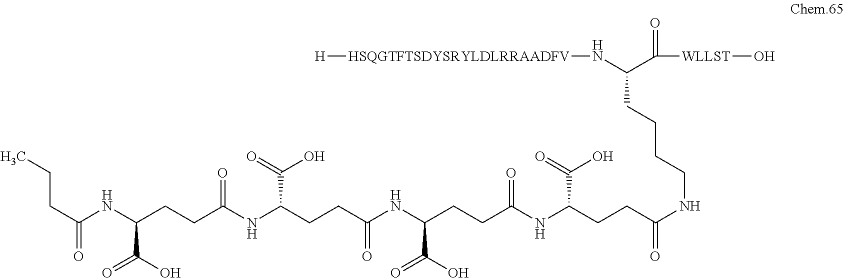

UPLC Method: UPLC_AP: Rt=7.31 min

### Example 66

### N^{ε24}-[(4S)-4-carboxy-4-[[(4S)-4-carboxy-4-[[(4S)-4-carboxy-4-[[(4S)-4-carboxy-4-(3-carboxypropanoylamino)butanoyl]amino]butanoyl]amino]butanoyl]amino]butanoyl]-[Arg¹²,Leu¹⁶,Ala²⁰,Lys²⁴,Leu²⁷,Ser²⁸]-Glucagon

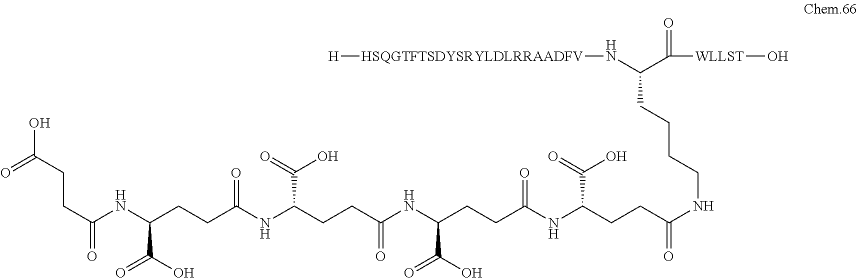

UPLC Method: UPLC_AP: Rt=7.22 min

### Example 67

### N^{ε24}-[(4S)-4-carboxy-4-[[(4S)-4-carboxy-4-[[(4S)-4-carboxy-4-[[(4S)-4-carboxy-4-(pentanoylamino)butanoyl]amino]butanoyl]amino]butanoyl]amino]butanoyl]-[Arg¹²,Leu¹⁶,Ala²⁰,Lys²⁴,Leu²⁷,Ser²⁸]-Glucagon

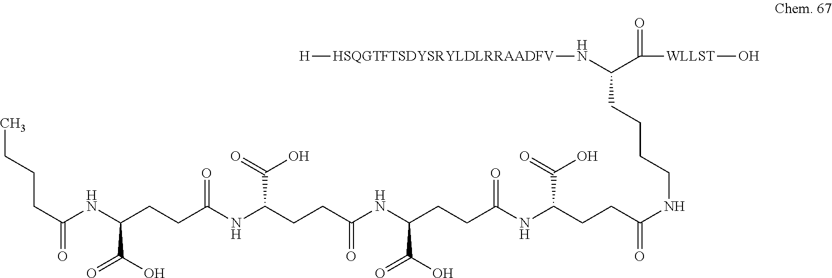

UPLC Method: UPLC_AP: Rt=7.38 min

### Example 68

### N^{ε16}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Lys16,Leu27,Ser28]-Glucagon

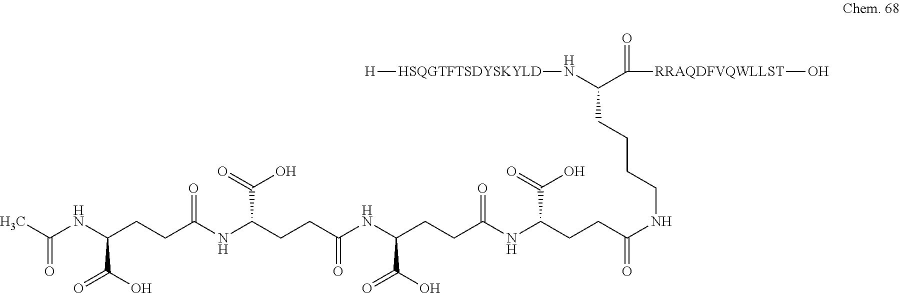

UPLC Method: UPLC_AP: Rt=6.50 min

### Example 69

### N^{ε21}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Lys21,Leu27,Ser28]-Glucagon

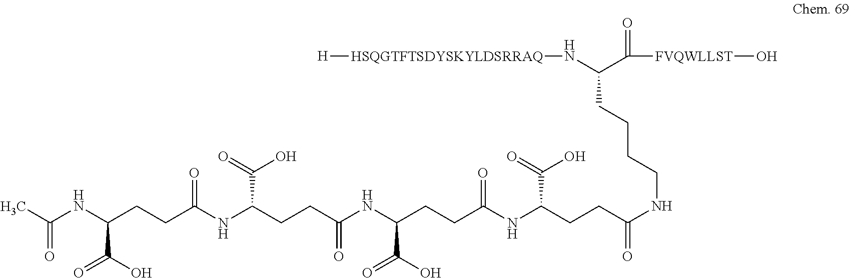

UPLC Method: UPLC_AP: Rt=6.41 min

### Pharmacological Methods

### Assay (I)

### Glucagon Activity

The glucagon receptor was cloned into HEK-293 cells having a membrane bound cAMP biosensor (ACTOne™). The cells (14000 per well) were incubated (37° C., 5% CO2) overnight in 384-well plates. Next day the cells were loaded with a calcium responsive dye that only distributed into the cytoplasm. Probenecid, an inhibitor of the organic anion transporter, was added to prevent the dye from leaving the cell. A PDE inhibitor was added to prevent formatted cAMP from being degraded. The plates were placed into a FLIPRTETRA and the glucagon analogues were added. End point data were collected after 6 minutes. An increase in intracellular cAMP was proportional to an increased in calcium concentrations in the cytoplasm. When calcium was bound the dry a fluorescence signal was generated. EC50-values were calculated in Prism5.

### Assay (II)

### ThT Fibrillation Assays for the Assessment of Physical Stability of Peptide Formulations

Low physical stability of a peptide may lead to amyloid fibril formation, which is observed as well-ordered, thread-like macromolecular structures in the sample eventually resulting in gel formation. This has traditionally been measured by visual inspection of the sample. However, that kind of measurement is very subjective and depending on the observer. Therefore, the application of a small molecule indicator probe is much more advantageous. Thioflavin T (ThT) is such a probe and has a distinct fluorescence signature when binding to fibrils [Naiki et al. (1989) Anal. BioChem. 177, 244-249; LeVine (1999) Methods. Enzymol. 309, 274-284].

The time course for fibril formation can be described by a sigmoidal curve with the following expression [Nielsen et al. (2001) BioChemistry 40, 6036-6046]:

$$F = {f}_{i} + {m}_{i} t + \frac{{f}_{f} + {m}_{f} t}{1 + {e}^{- [ ( t - {t}_{0} ) / τ ]}} \qquad Eq . ( 1 )$$

Here, F is the ThT fluorescence at the time t. The constant t0 is the time needed to reach 50% of maximum fluorescence. The two important parameters describing fibril formation are the lag-time calculated by t0−2τ and the apparent rate constant kapp 1/τ.

Formation of a partially folded intermediate of the peptide is suggested as a general initiating mechanism for fibrillation. Few of those intermediates nucleate to form a template onto which further intermediates may assembly and the fibrillation proceeds. The lag-time corresponds to the interval in which the critical mass of nucleus is built up and the apparent rate constant is the rate with which the fibril itself is formed.

Samples were prepared freshly before each assay. Each sample composition is described in the legends. The pH of the sample was adjusted to the desired value using appropriate amounts of concentrated NaOH and HCl. Thioflavin T was added to the samples from a stock solution in H₂O to a final concentration of 1 μM.

Sample aliquots of 200 μl were placed in a 96 well microtiter plate (Packard OptiPlate™-96, white polystyrene). Usually, four or eight replica of each sample (corresponding to one test condition) were placed in one column of wells. The plate was sealed with Scotch Pad (Qiagen).

Incubation at given temperature, shaking and measurement of the ThT fluorescence emission were done in a Fluoroskan Ascent FL fluorescence platereader (Thermo Labsystems). The temperature was adjusted to the desired value, typically 30° C. or 37° C. The plate was either incubated without shaking (no external physical stress) or with orbital shaking adjusted to 960 rpm with an amplitude of 1 mm. Fluorescence measurement was done using excitation through a 444 nm filter and measurement of emission through a 485 nm filter.

Each run was initiated by incubating the plate at the assay temperature for 10 min. The plate was measured every 20 minutes for a desired period of time. Between each measurement, the plate was shaken and heated as described.

After completion of the ThT assay the four or eight replica of each sample was pooled and centrifuged at 20000 rpm for 30 minutes at 18° C. The supernatant was filtered through a 0.22 μm filter and an aliquot was transferred to a HPLC vial.

The concentration of peptide in the initial sample and in the filtered supernatant was determined by reverse phase HPLC using an appropriate standard as reference. The percentage fraction the concentration of the filtered sample constituted of the initial sample concentration was reported as the recovery.

The measurement points were saved in Microsoft Excel format for further processing and curve drawing and fitting was performed using GraphPad Prism. The background emission from ThT in the absence of fibrils was negligible. The data points are typically a mean of four or eight samples and shown with standard deviation error bars. Only data obtained in the same experiment (i.e. samples on the same plate) are presented in the same graph ensuring a relative measure of fibrillation between experiments.

The data set may be fitted to Eq. (1). However, the lag time before fibrillation may be assessed by visual inspection of the curve identifying the time point at which ThT fluorescence increases significantly above the background level.

### Assay (III)

### ThT Fibrillation and Solubility at Different pH

Glucagon analogue was dissolve to 250 μM and aliquots were adjusted to different pH. Samples equilibrated at room temperature for two-four days and were subsequently centrifuged. Concentration of peptide in solution centrifugation is shown versus the pH measured after equilibration. Native human glucagon shown in black and with closed squares, glucagon analogue in light grey with open squares.

Furthermore, 200 μl from each pH adjusted and equilibrated sample were removed after the centrifugation, and transferred to a white 96 well microtiter plate (Optiplate, Packard). Amyloid fibril indicator Thioflavin T (ThT) was added to 1 μM. This plate was sealed and incubated in a Fluoroskan Ascent FL fluorescence platereader (Thermo Labsystems) at 37° C. and with orbital shaking adjusted to 960 rpm with an amplitude of 1 mm. Fluorescence measurement was done using excitation through a 444 nm filter and measurement of emission through a 485 nm filter. Each run was initiated by incubating the plate at the assay temperature for 10 min. The plate was measured every 20 minutes in 45 hours. Between each measurement, the plate was shaken and heated as described. The lag time before any increase in ThT fluorescence (i.e. amyloid fibril formation) is depicted on the graph using the right y-axis and the broken light grey line and triangles. Absence of increased ThT fluorescence was noted as a lag time of 45 hours. In few points within the precipitation zone no increase in ThT fluorescence was observed, but this was ascribed to the fact that all peptide was precipitated and thus no value for lag time is depicted for these point.

TABLE 1
In vitro data on receptor binding, ThT assay lag time and recovery.

| | Assay (I) | | |
|---|---|---|---|
| | Glucagon | ThT assay | ThT assay |
| | [EC50] | [Lag time] | [Recovery] |
| Compound | (nM) | (h) | (%) |
| Native glucagon* | 0.010 | 1.5 | 2.5 |
| [28D, 30E]Glucagon* | 0.010 | 3 | 13 |
| [30E]Glucagon* | 0.012 | 0.3 | 7 |
| Example 1* | 0.0073 | 6 | 23 |
| Example 2 | 0.0040 | 45 | 100 |
| Example 3 | 0.0105 | 45 | 100 |
| Example 4 | 0.0145 | 45 | 100 |
| Example 5 | 0.0563 | 45 | 100 |
| Example 6 | 0.0795 | 45 | 100 |
| Example 7 | 0.0243 | 45 | 100 |
| Example 8 | 0.0269 | 2 | 78 |
| Example 9 | 0.0370 | 45 | 100 |
| Example 10 | 0.0167 | 45 | 100 |
| Example 11 | 0.0124 | 45 | 100 |
| Example 12 | 0.0103 | 45 | 100 |
| Example 13 | 0.0066 | 38 | 100 |
| Example 14* | 0.0082 | 6 | 0 |
| Example 15* | 0.0083 | 2.7 | 0 |
| Example 16 | 0.0065 | 45 | 100 |
| Example 17 | 0.0165 | 45 | 100 |
| Example 18 | 0.0045 | 3 | 14 |
| Example 19 | 0.0145 | 45 | 100 |
| Example 20 | 0.0050 | 3 | 8 |
| Example 21 | 0.0055 | 26 | 100 |
| Example 22 | 0.0055 | 45 | 100 |
| Example 23 | 0.0105 | 45 | 100 |
| Example 24 | 0.0055 | 4 | 70 |
| Example 25 | 0.0170 | 34 | 96 |
| Example 26 | 0.0120 | 45 | 100 |
| Example 27 | 0.0960 | 45 | 100 |
| Example 28 | 0.4690 | 45 | 100 |
| Example 29 | 0.2070 | 45 | 100 |
| Example 30 | 0.0065 | 45 | 96 |
| Example 31 | 0.0130 | 45 | 100 |
| Example 32 | 0.0090 | 45 | 96 |
| Example 33 | 0.0080 | 45 | 100 |
| Example 34 | 0.0023 | 45 | 100 |
| Example 35 | 0.0028 | 45 | 100 |
| Example 36 | 0.0025 | 45 | 100 |
| Example 37 | 0.0030 | 45 | 87 |
| Example 38 | 0.0029 | 45 | 100 |
| Example 39 | 0.0030 | 45 | 99 |
| Example 40 | 0.0030 | 45 | 110 |
| Example 41 | 0.0030 | 45 | 100 |
| Example 42 | 0.0080 | 45 | 100 |
| Example 43 | 0.0160 | 45 | 100 |
| Example 44 | 0.0750 | 45 | 100 |
| Example 45 | 0.0270 | 45 | 100 |
| Example 46 | 0.0240 | 45 | 100 |
| Example 47 | 0.0090 | 25 | 100 |
| Example 48 | 0.0050 | 14 | 100 |
| Example 49 | 0.0540 | 45 | 100 |
| Example 50 | 0.1530 | n.d. | n.d. |
| Example 51 | 0.0070 | 35 | 98 |
| Example 52 | 0.0100 | 45 | 100 |
| Example 53 | 0.0070 | 10 | 87 |
| Example 54 | 0.0180 | 45 | 100 |
| Example 55 | 0.0510 | 45 | 100 |
| Example 56 | 0.0200 | 45 | 100 |
| Example 57 | 0.0170 | 45 | 100 |
| Example 58 | 0.003 | 45 | 105 |
| Example 59 | 0.013 | 45 | 96 |
| Example 62 | 0.052 | 45 | 100 |
| Example 65 | 0.021 | 45 | 104 |
| Example 66 | 0.020 | 45 | 104 |
| Example 67 | 0.022 | 45 | 104 |

*compound used as control

### Assay (IV)

PK/PD in GöTtingen Minipigs after SC Dosing

Male Göttingen minipigs (Ellegaard Göttingen Minipigs NS, Dalmose, Denmark), approximately 7-10 months of age and weighing from approximately 15-20 kg, were used in the studies. The minipigs were housed individually and fed restrictedly once daily with SDS minipig diet (Special Diets Services, Essex, UK). After at least 2 weeks of acclimatisation two permanent central venous catheters were implanted in vena cava caudalis or cranialis in each animal to be able to obtain stress free blood samples. During the anaesthesia for placement of permanent intravenous catheters, the pigs had been scanned on the side of the neck, and an area with no underlying muscle suitable for subcutaneous injection was marked by a tatoo to ensure that the compounds were delivered subcutaneously in the same place each time. The animals were allowed 1 week recovery after the surgery, and were then used for repeated pharmacokinetic studies with a suitable wash-out period between dosings.

The animals were fasted for approximately 18 h before dosing and during the whole study, but had ad libitum access to water at all times. GlucaGen® HypoKit (NNC0025-8000) was dosed subcutaneously (SC) to 8 Göttingen minipigs (3.5 nmol/kg) and NNC0025-8000 dissolved in lactose solution 107 mg/ml was dosed IV to 8 Göttingen minipigs (2 nmol/kg).

The glucagon derivative of Example 2 was dissolved in 50 mM sodium phosphate, 145 mM sodium chloride, 0.05% tween 80, pH 7.4 and was dosed IV to 2 pigs (2 nmol/kg) and SC to 4 pigs (3.5 nmol/kg). The concentration of compound in the dosing solutions for IV and SC dosing was 60 and 287 nmol/mL, respectively.

Blood was sampled at predefined time points for up to 4 hours post dosing. Blood samples were collected in EDTA buffer (8 mM) with aprotinin (14 μM) and then centrifuged at 4° C. and 1942 G for 10 minutes. Plasma was transferred to Micronic tubes on dry ice, and kept at −20° C. until analyzed for plasma concentration of the respective glucagon derivative using ELISA or a similar antibody based assay. 10 μL of plasma was transferred into 500 μL EBIO solution and measured on a Biosen auto analyzer (BIOSEN S_Line, EKF Diagnostics, Cardiff, UK) according to the manufacturer's instructions.

Three pigs in the SC GlucaGen® HypoKit group and 1 pig in the SC group dosed with Example 2 were not dosed correctly and were therefore omitted from the graphical presentation of data and from pharmacokinetic analysis

Individual plasma concentration-time profiles were analyzed by a non-compartmental model in WinNonlin v. 5.0 (Pharsight Inc., Mountain View, Calif., USA), and selected pharmacokinetic parameters are given in Table 2, below. Glucose data were compared using two-way ANOVA and selected PK parameters were compared with t-test or nonparametric test (GraphPad Prism version 5.00 for Windows, GraphPad Software, San Diego Calif. USA).

TABLE 2
Pharmacokinetic parameters obtained after SC dosing of GlucaGen ® HypoKit
and Example 2 to Göttingen minipigs.

| | Bioavailability | Tₘₐₓ | Cₘₐₓ/Dose | T_{1/2} | Tmax Glucose |
|---|---|---|---|---|---|
| | (mean ± SD) | (median) | (mean ± SD) | (harmonic mean) | (median) |
| Compound | (%) | [range] (min) | (kg*pmol/L/pmol) | [range] (min) | [range] (min) |
| GlucaGen ® | 26 ± 9 | 9 [6-12] | 0.28 ± 0.10 | 23.4 [17.5-29.8] | 15 [9-20] |
| Hypokit | | | | | |
| (n = 5) | | | | | |
| Example 2 | 91 ± 13 *** | 40 [20-75] * | 1.26 ± 0.11 *** | 64.0 [60.4-70.7] *** | 12 [9-20] |
| (n = 3) | | | | | |

* p < 0.05,
*** p < 0.001

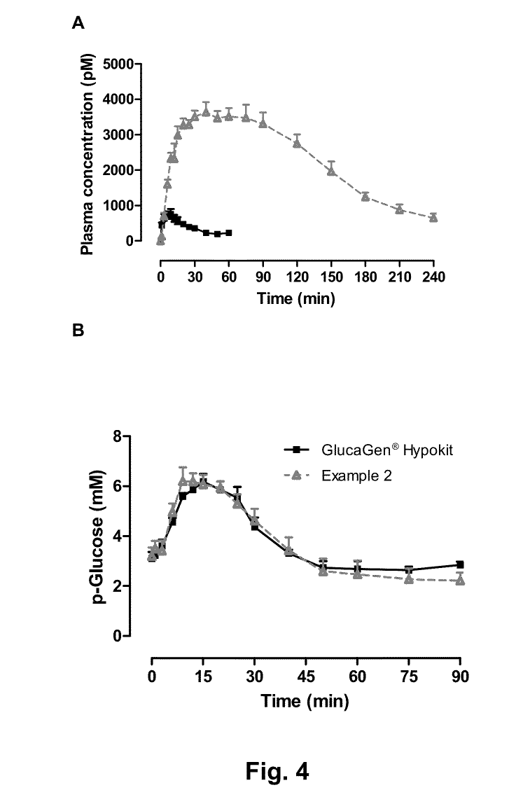

The pig studies with Example 2 show that an equivalent pharmacodynamic profile compared to that of the GlucaGen® HypoKit can be obtained in this model in spite of significant differences in pharmacokinetic parameters (FIG. 4, Table 2).

### Assay (V)

### Long-Term Storage Stability

The examples 2, 4, 5, 7, 12, were all formulated as 250 μM peptide, 184 mM propyleneglycol, 58 mM phenol, 8 mM disodium phosphate pH 7.4. The Aib analogue was formulated as 125 μM peptide, 184 mM propyleneglycol, 58 mM phenol, 8 mM disodium phosphate pH 7.4.

Of these formulations 1 ml was filled in a 1.5 ml Penfill®.

Penfill® were stored quiescently at 5° C. (for 24 weeks), 25° C. (for 4 weeks, 8 weeks, 16 weeks, 24 weeks) and at 37° C. (4 weeks).

At each time point a Penfill® was withdrawn, visually inspected using a 10.000 lux light source. The turbidity was measured by placing the Penfill® in a Hach 2100AN turbidimeter. These observations are summarised in Table 2.

All examples, except the Aib analogue I, in this formulation remained visually transparent at all measurement times, i.e. for 24 weeks at both 5° C. and 25° C. and for 4 weeks at 37° C. The Aib analogue, however, precipitated after 16 weeks at 25° C. and, moreover, after 24 weeks at 5° C. This was observed visually even at ordinary day light and by high turbidity NTU readings.

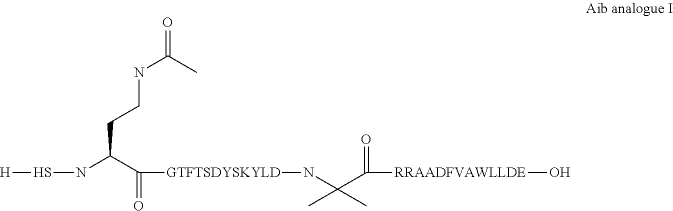

TABLE 3
NTU (Nephelometric Turbidity Units): A single Penfill ® was measured at
each time point. Values at “Start” are minimum and maximum values of all Penfill ® of
the formulated peptide. ¹⁾and ²⁾denote two individual Penfill ®, ¹⁾observed after 16 w
and withdrawn, ²⁾observed at 16 w and after 24 w before withdrawal. ³⁾Too many air
bubbles were present disabling a meaningful turbidity reading.

| | | | 24 w @ | 4 w @ | 4 w @ | 8 w @ | 16 w @ | 24 w @ |
|---|---|---|---|---|---|---|---|---|
| Analogue | | Start | 5° C. | 37° C. | 25° C. | 25° C. | 25° C. | 25° C. |
| Example 2 | Visual insp. | Clear | Clear | Clear | Clear | Clear | Clear | Clear |
| | NTU | 0.304-0.536 | 0.529 | 0.378 | 0.338 | 0.404 | 0.431 | 0.387 |
| Example 4 | Visual insp. | Clear | Clear | Clear | Clear | Clear | Clear | Clear |
| | NTU | 0.339-0.557 | 0.535 | 0.374 | 0.394 | 0.493 | 0.494 | 0.477 |
| Example 5 | Visual insp. | Clear | Clear | Clear | Clear | Clear | Clear | Clear |
| | NTU | 0.362-0.622 | 0.393 | 0.409 | 0.442 | 0.427 | 0.491 | 0.455 |
| Example 7 | Visual insp. | Clear | Clear | Clear | Clear | Clear | Clear | Clear |
| | NTU | 0.443-0.741 | n.a³⁾ | 0.586 | 0.542 | 0.496 | 0.586 | 0.473 |
| Example 12 | Visual insp. | Clear | Clear | Clear | Clear | Clear | Clear | Clear |
| | NTU | 0.378-0.711 | 0.599 | 0.544 | 0.463 | 0.384 | 0.343 | 0.363 |
| Aib | VVisual | Clear | Precipitate | Clear | Clear | Clear | Precipitate¹⁾ | Precipitate²⁾ |
| analogue I* | insp. | | | | | | Precipitate²⁾ | |
| | NTU | 0.382-0.668 | 2.52 | 0.477 | 0.548 | 0.473 | 0.567¹⁾ | 2.21²⁾ |
| | | | | | | | 1.82²⁾ | |

*Compound included as reference; w: Weeks, Visual insp.: Visual inspection, Precipitate: denotes visual identification of precipitated material in the Penfill. In all instances here, precipitate was visual even in ordinary day light without using the 10.000 lux light source.

### Stability of Peptide Secondary Structure During Long-Term Storage

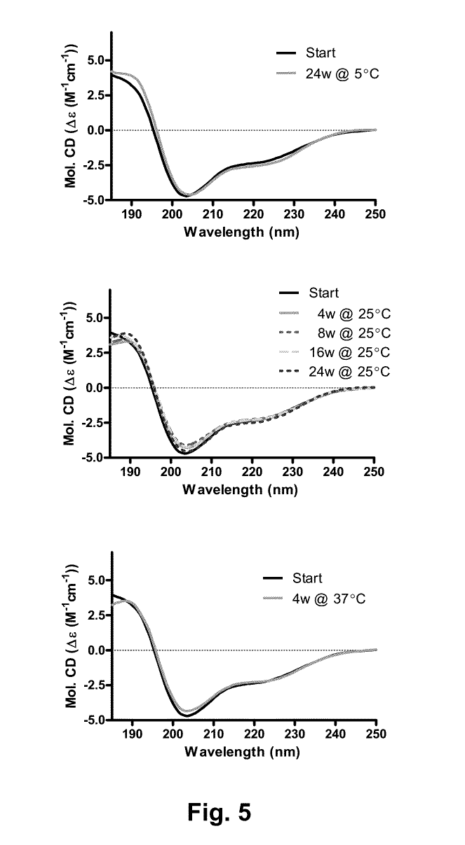

Example 5 was formulated as 250 μM peptide, 184 mM propyleneglycol, 8 mM disodium phosphate pH 7.4. This formulation was filled and stored as described in Assay (V) at various temperatures and time intervals. Aliquots were withdrawn at the indicated time points and far UV circular dichroism (CD) spectra were recorded. Background using formulation vehicle alone was subtracted, and the depicted molecular CD spectra had been normalised using measured peptide concentration and the number of peptide bonds and the light path length of 0.01 cm. Various combinations of the spectra are shown in FIG. 5.

Far UV CD spectroscopy is sensitive to the secondary structure and folding of the peptide chain. All the recorded CD spectra of example 5 at various storage conditions are virtually identical. This indicates the secondary structure of example 5 remains stable and intact throughout the storage. Moreover, the shape of the CD spectra indicates a substantial amount of alfa helical conformation and the spectra are devoid of the characteristics of a peptide folded as a beta sheet [Manavalan and Johnson, Nature 305, 831-832, 1983], which is pronounced in amyloid fibrils. These observations indicate a high physical stability of example 5 resulting in a low propensity for forming amyloid fibrils during prolonged storage.

1. A glucagon peptide derivative of formula [I]:
His-X₂-X₃-Gly-Thr-Phe-Thr-Ser-Asp-X₁₀-Ser-X₁₂-Tyr-Leu-X₁₅-X₁₆-Arg-X₁₈-Ala-X₂₀-X₂₁-Phe-Val-X₂₄-Trp-Leu-X₂₇-X₂₈-X₂₉-X₃₀ [I]
comprising a substituent attached to a nitrogen of a side chain of an amino acid selected from the group consisting of positions X₁₂, X₁₆, X₂₀, X₂₁, X₂₄, X₂₈, X₂₉, and X₃₀ of said glucagon peptide, and wherein said substituent comprises formula II:
Y₁-Y₂-Y₃-Y₄-Y₅-Y₆-Y₇-Y₈-Y₉-Y₁₀-Y₁₁-Y₁₂ [II]
wherein
Y₁, Y₂, Y₃, Y₄, Y₅, Y₆, Y₇, Y₈, Y₉, Y₁₀ and Y₁₁ are individually absent, individually represent an amino acid or i, ii, iii, iv, which have the stereochemistry L or D, or the structure v: 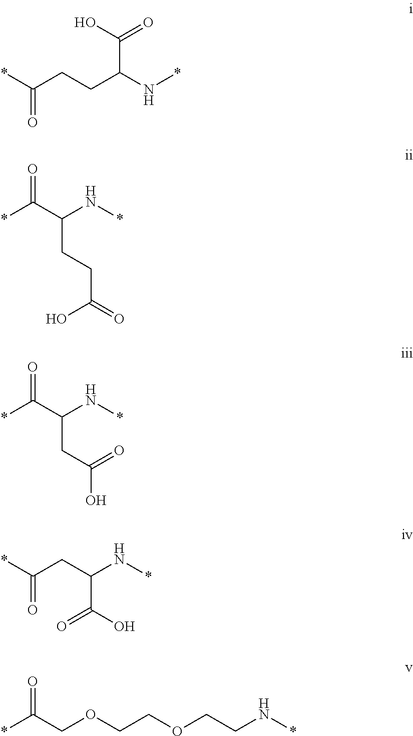
and,
Y₁₂ is absent or represents a C₂₋₆ acyl group or a succinoyl moiety provided that the substituent of formula II contains between three and ten negatively charged moieties, or a pharmaceutically acceptable salt, amide or carboxylic acid thereof.

2. The glucagon peptide derivative according to claim 1, wherein
Y₁ is absent or represents an amino acid, selected from the group consisting of Arg, ε-Lys and Gly;
Y₂, Y₃, Y₄, Y₅, Y₆, Y₇, Y₈, Y₉, Y₁₀ and Y₁₁ are individually absent or individually represent an amino acid or i or ii; 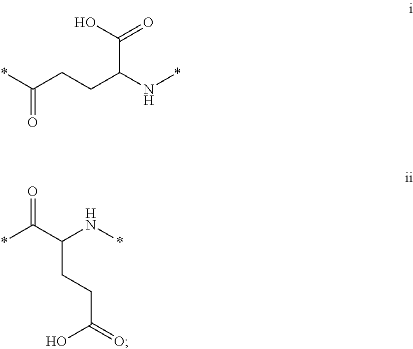 [SMILES: *NC(CCC(*)=O)C(=O)O ; *NC(CCC(=O)O)C(*)=O]
and
Y₁₂ is absent or represents a structure of the formula selected from the group consisting of vi, vii, viii, ix, x and xi:

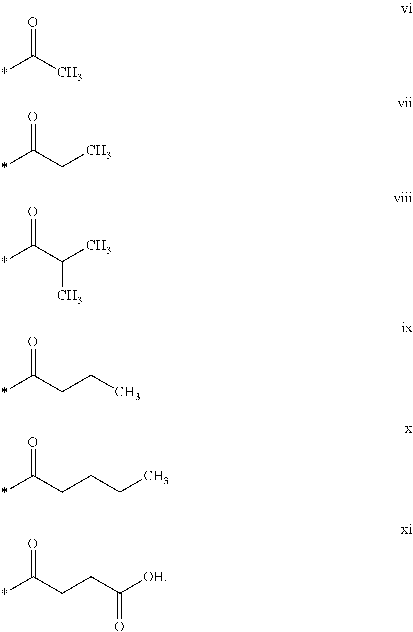

3. The glucagon peptide derivative according to claim 2, wherein, wherein:
Y₁ is absent or represents an amino acid selected from the group consisting of Arg, ε-Lys and Gly;
Y₂, Y₃, Y₄, Y₅, Y₆, Y₇, Y₈, Y₉, Y₁₀ and Y₁₁ is individually absent or individually represents i or ii; 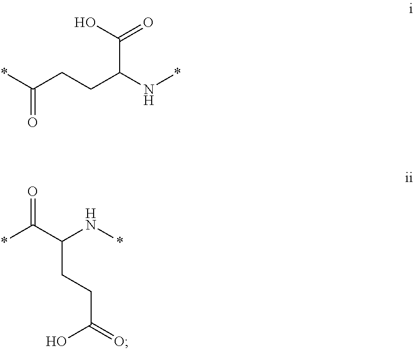 [SMILES: *NC(CCC(*)=O)C(=O)O ; *NC(CCC(=O)O)C(*)=O]
and
Y₁₂ is absent or represents a structure of the formulae selected from the group consisting of vi, vii, viii, ix, x and xi:

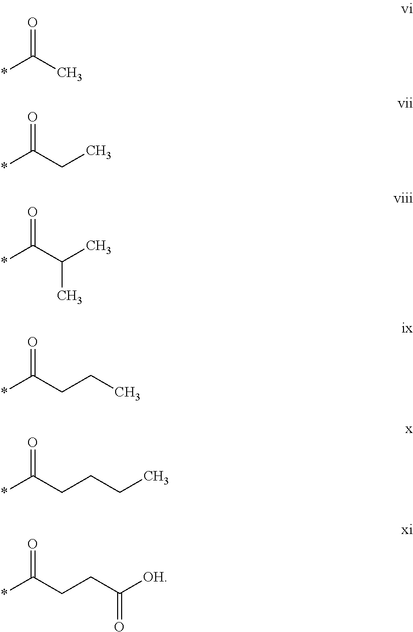

4. The glucagon peptide derivative according to claim 1, wherein said substituent is attached to the side chain of the amino acid in position X₂₄ of said glucagon peptide.

5. The glucagon peptide derivative according to claim 4, wherein X₂₄ represents Lys.

6. The glucagon peptide derivative according to claim 1, wherein said glucagon peptide comprises up to 15 amino acid residue substitutions and wherein:
X₂ represents Ser, Aib, Thr, Ala, or Gly;
X₃ represents Gln, His;
X₁₀ represents Tyr, Val or;
X₁₂ represents Lys, Orn or Arg;
X₁₅ represents Asp or Glu;
X₁₆ represents Ser, Thr, Lys, Val, Tyr, Phe, Leu, Ile or Trp or Orn;
X₁₈ represents Arg, Lys, Ala or Orn;
X₂₀ represents Gln, Lys, Ala, Glu or Orn;
X₂₁ represents Asp, Glu, Lys or Orn;
X₂₄ represents Gln, Lys, or Orn;
X₂₇ represents Met or Leu
X₂₈ represents Asn, Lys, Ser or Orn;
X₂₉ represents Thr, Lys or Orn and
X₃₀ is absent or represents Lys Pro or Orn.

7. The glucagon peptide according to claim 1, selected from the group consisting of:
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Lys24,Leu27]-Glucagon 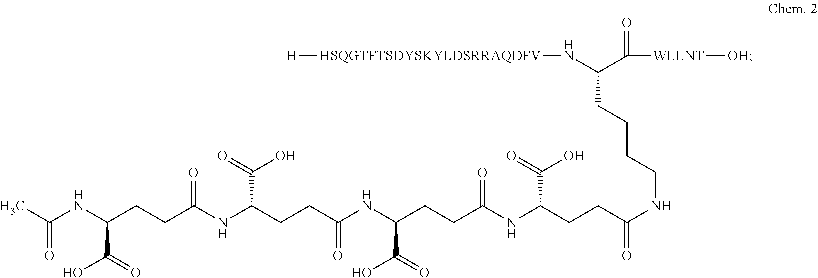
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[His3,Lys24,Leu27,Ser28]-Glucagon 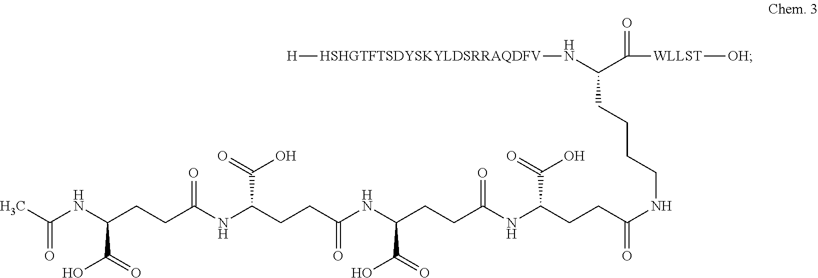
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[His3,Ala20,Lys24,Leu27,Ser28]-Glucagon 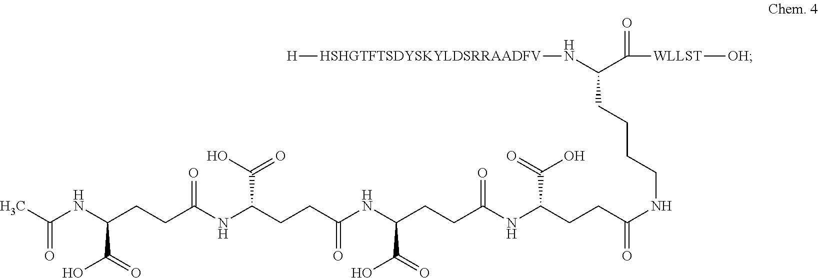
N^{ε24}-[(4S)-4-[[(4S)-4-[[(4S)-4-[[(4S)-4-amino-4-carboxybutanoyl]amino]-4-carboxybutanoyl]amino]-4-carboxybutanoyl]amino]-4-carboxybutanoyl]-[His3,Glu15,Ala20,Lys24,Leu27,Ser28]-Glucagon 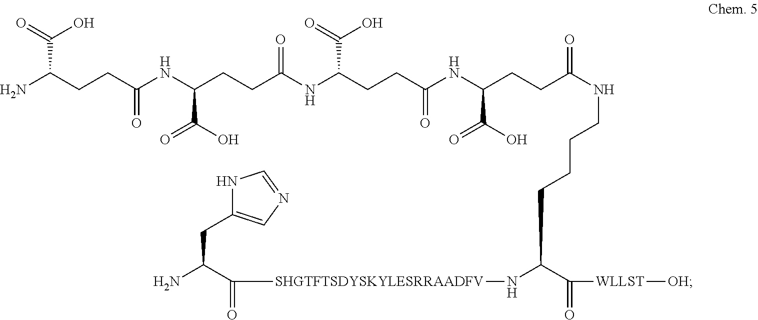
N^{ε24}-[(4S)-4-[[(4S)-4-[[(4S)-4-[[(4S)-4-amino-4-carboxybutanoyl]amino]-4-carboxybutanoyl]amino]-4-carboxybutanoyl]amino]-4-carboxybutanoyl]-[His3,Arg12,Glu15,Ala20,Lys24,Leu27,Ser28]-Glucagon 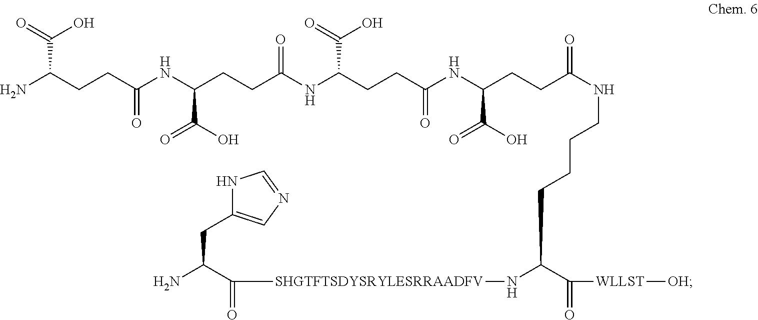
N^{ε24}-[(4S)-4-[[(4S)-4-[[(4S)-4-[[(4S)-4-amino-4-carboxybutanoyl]amino]-4-carboxybutanoyl]amino]-4-carboxybutanoyl]amino]-4-carboxybutanoyl]-[His3,Thr16,Ala20,Lys24,Leu27,Ser28]-Glucagon 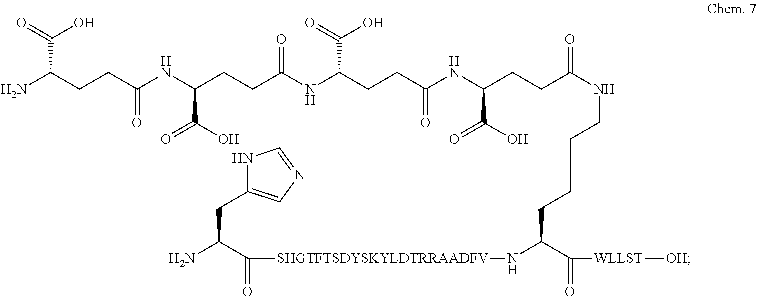
N^{ε24}-[(4S)-4-[[(4S)-4-[[(4S)-4-[[(4S)-4-amino-4-carboxybutanoyl]amino]-4-carboxybutanoyl]amino]-4-carboxybutanoyl]amino]-4-carboxybutanoyl]-[His3,Arg12,Ile16,Ala20,Lys24,Leu27,Ser28]-Glucagon 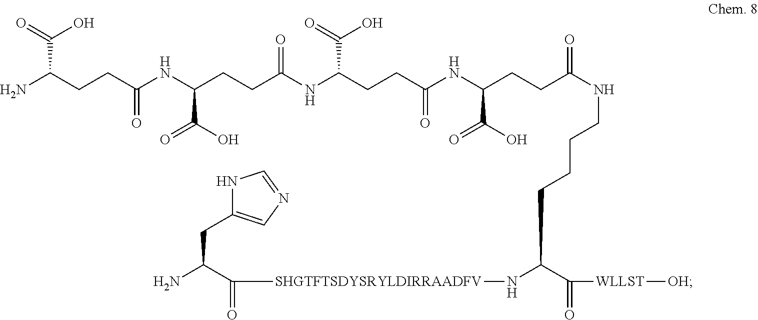
N^{ε24}-[(4S)-4-[[(4S)-4-[[(4S)-4-[[(4S)-4-amino-4-carboxybutanoyl]amino]-4-carboxybutanoyl]amino]-4-carboxybutanoyl]amino]-4-carboxybutanoyl]-[His3,Arg12,Thr16,Ala20,Lys24,Leu27,Ser28]-Glucagon 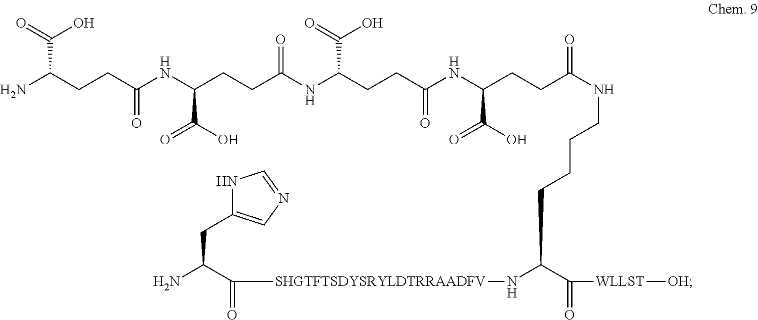
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[His3,Val16,Ala20,Lys24,Leu27,Ser28]-Glucagon 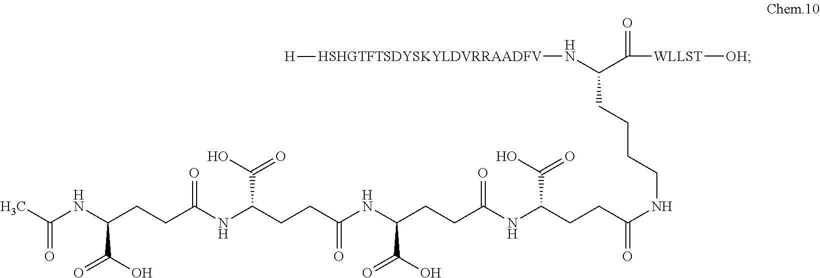
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[His3,Ile16,Ala20,Lys24,Leu27,Ser28]-Glucagon 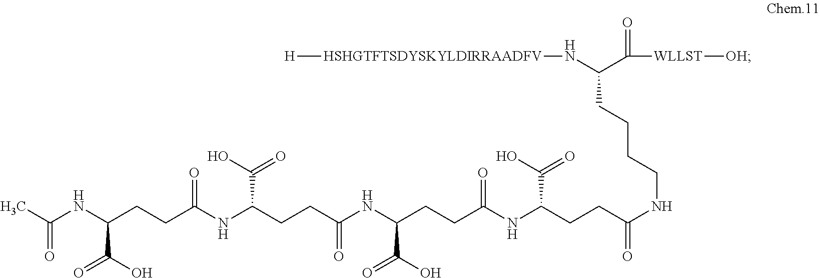
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[His3,Phe16,Ala20,Lys24,Leu27,Ser28]-Glucagon 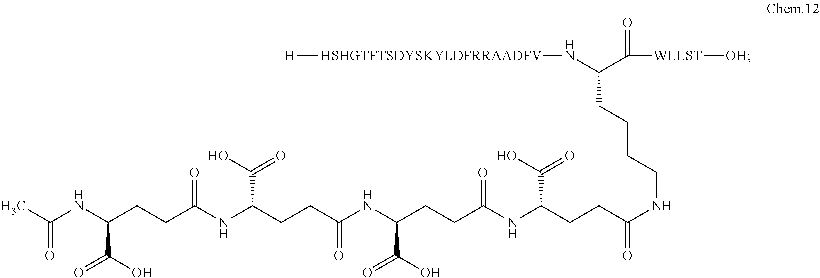
N^{ε24}[(4S)-4-[[(4S)-4-[[(4S)-4-acetamido-4-carboxybutanoyl]amino]-4-carboxybutanoyl]amino]-4-carboxybutanoyl]-[Lys24,Leu27]-Glucagon 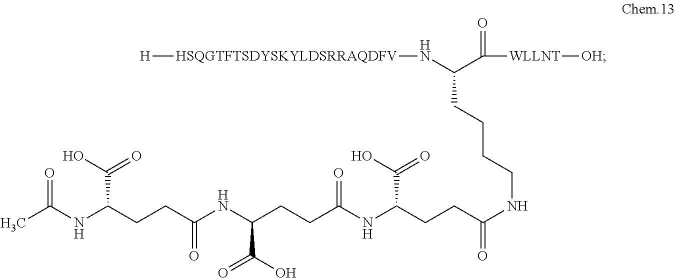
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Val16,Lys24,Leu27]-Glucagon 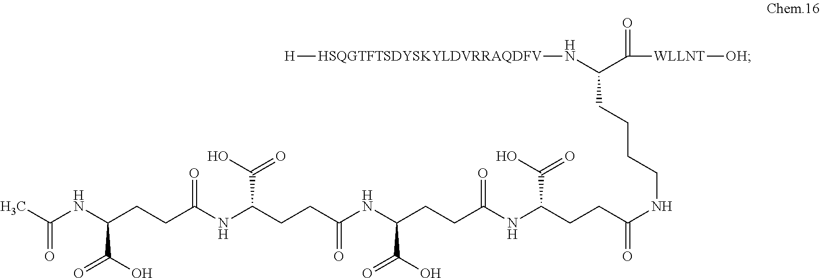
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Glu15,Lys24,Leu27]-Glucagon 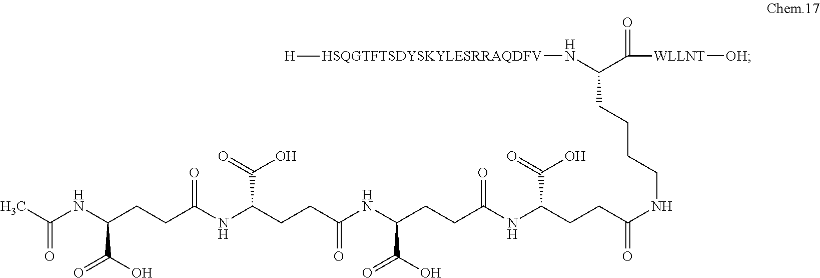
N^{ε24}-[(2S)-2-[[(2S)-2-[[(2S)-2-acetamido-4-carboxybutanoyl]amino]-4-carboxybutanoyl]amino]-4-carboxybutanoyl]-[Lys24,Leu27]-Glucagon 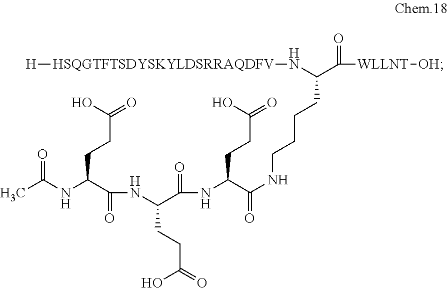
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[His3,Lys24,Leu27]-Glucagon 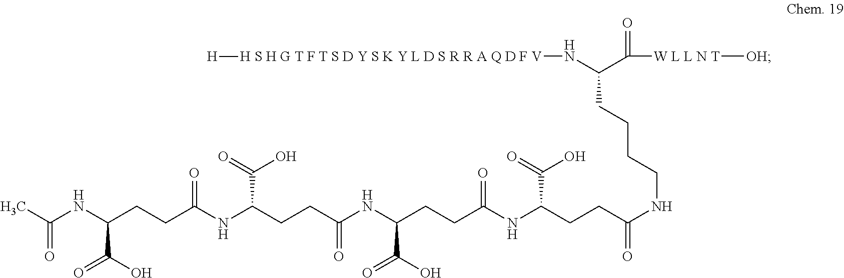
N^{ε24}-[(2S)-2-[[(2S)-2-[[(2S)-2-amino-4-carboxybutanoyl]amino]-4-carboxybutanoyl]amino]-4-carboxybutanoyl]-[Lys24,Leu27]-Glucagon 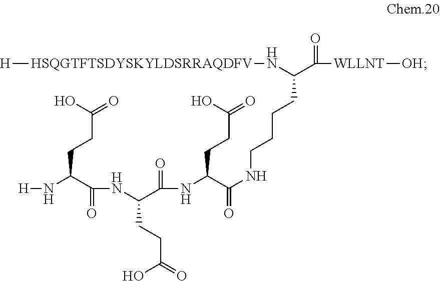
N^{ε24}-[(2S)-2-[[(2S)-2-[[(2S)-2-[[(2S)-2-amino-4-carboxybutanoyl]amino]-4-carboxybutanoyl]amino]-4-carboxybutanoyl]amino]-4-carboxybutanoyl]-[Lys24,Leu27]-Glucagon 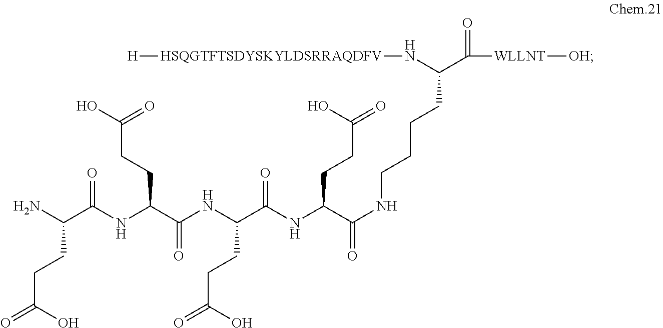
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Glu15,Glu21,Lys24,Leu27,Ser28]-Glucagon 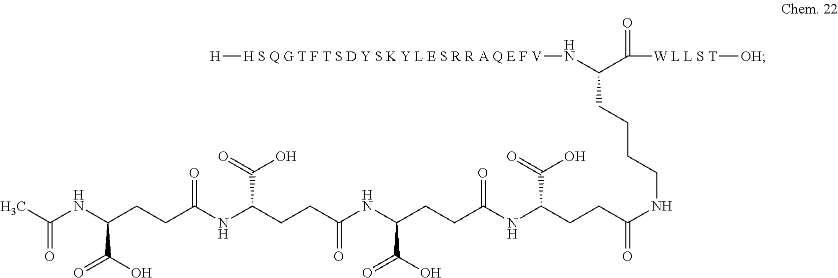
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Glu15,Lys24, Leu27,Ser28]-Glucagon 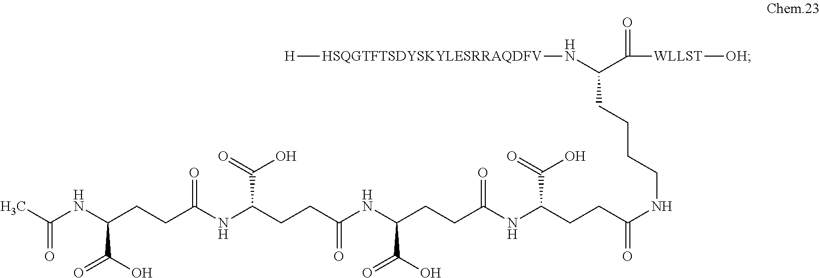
N^{ε24}-[(4S)-4-[[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]amino]-4-carboxybutanoyl]-[Lys24,Leu27]-Glucagon 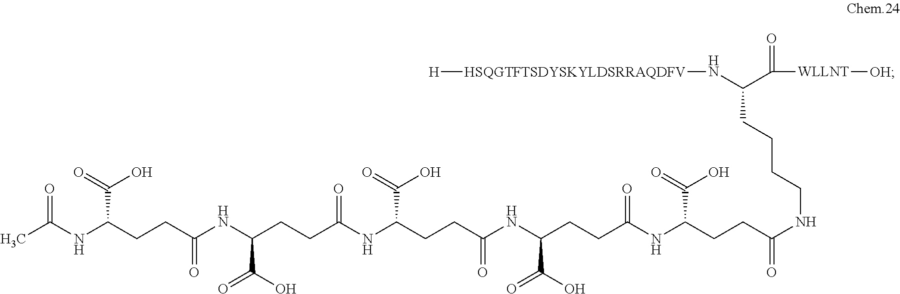
N^{ε24}-[(4S)-4-[[(4S)-4-[[(4S)-4-[[(4S)-4-[[(4S)-4-[[(4S)-4-[[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]amino]-4-carboxybutanoyl]amino]-4-carboxybutanoyl]amino]-4-carboxybutanoyl]amino]-4-carboxybutanoyl]amino]-4-carboxybutanoyl]amino]-4-carboxybutanoyl]-[Lys24,Leu27]-Glucagon 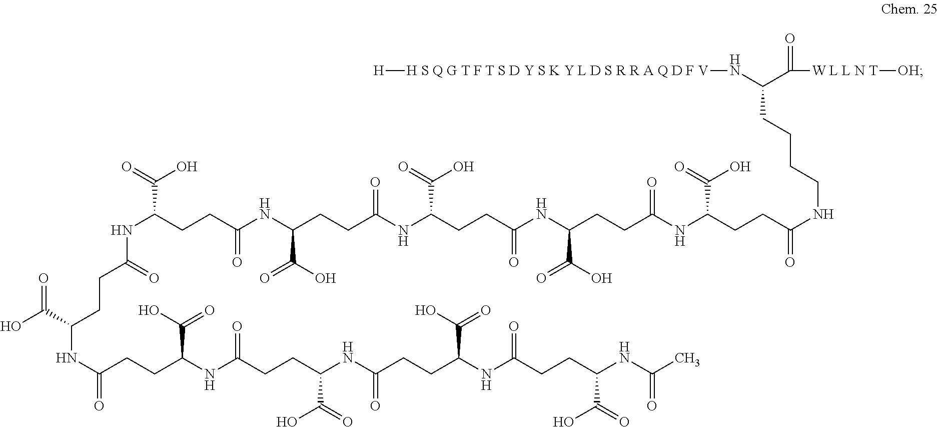
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Arg12,Thr16,Lys24,Leu27,Ser28]-Glucagonyl-Pro 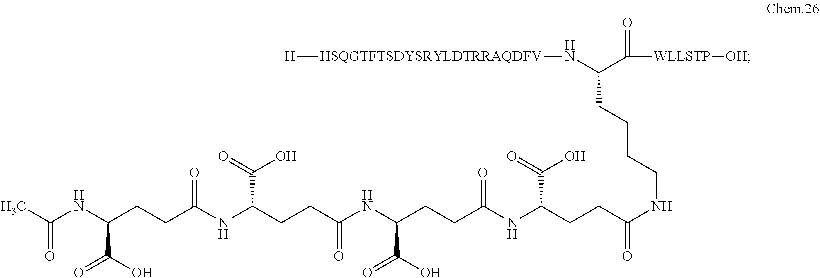
N^{ε12}-[(2S)-6-[[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]amino]-2-aminohexanoyl]-[Leu27]-Glucagon 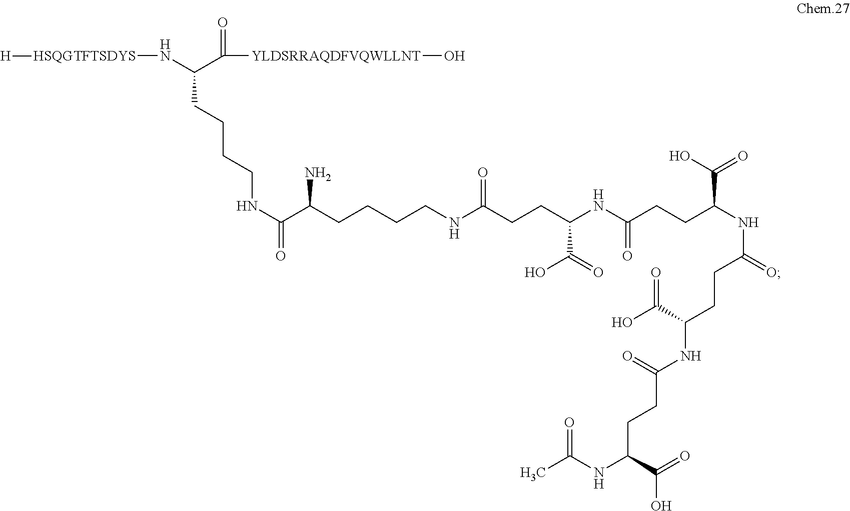
N^{ε12}-[2-[[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]amino]acetyl]-[Leu27]-Glucagon 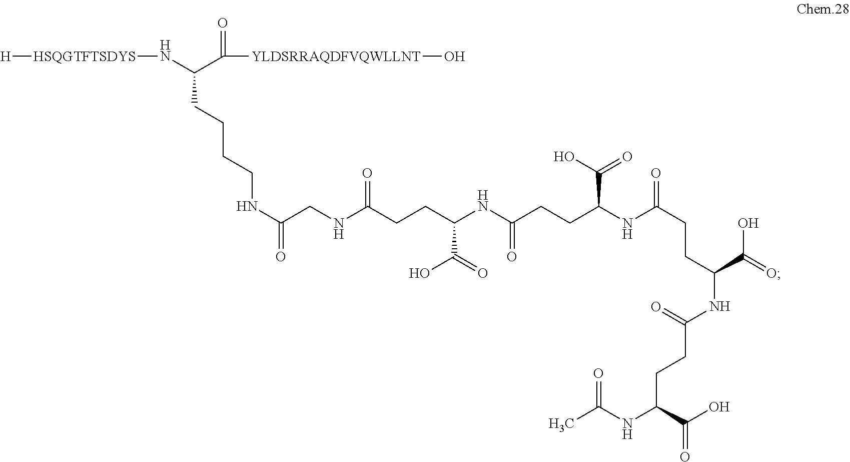
N^{ε12}-[(2S)-2-[[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]amino]-5-carbamimidamidopentanoyl]-[Leu27]-Glucagon 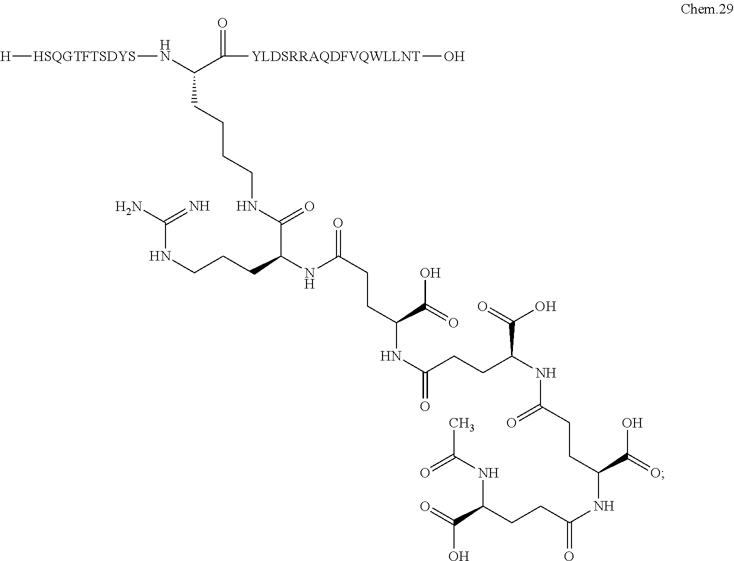
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Thr16,Lys24,Leu27,Ser28]-Glucagon 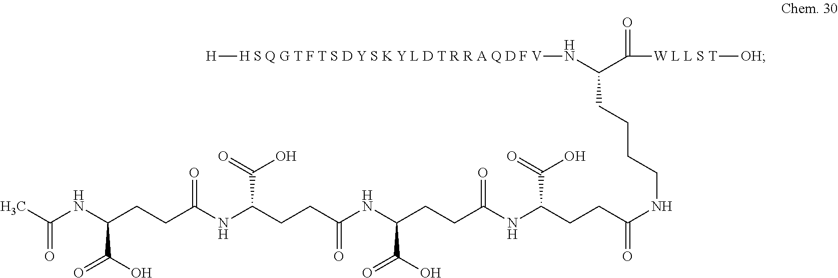
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[His3,Glu15,Thr16,Lys24,Leu27,Ser28]-Glucagon 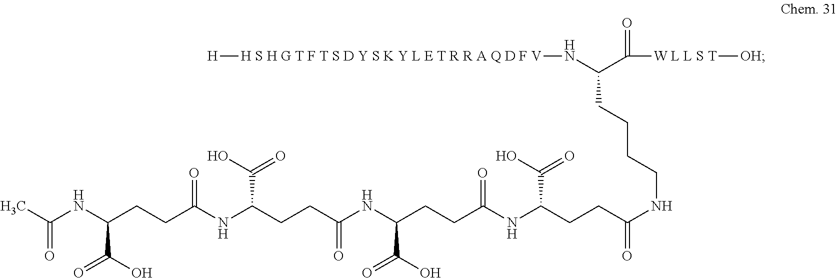
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[His3,Thr16,Lys24,Leu27,Ser28]-Glucagon 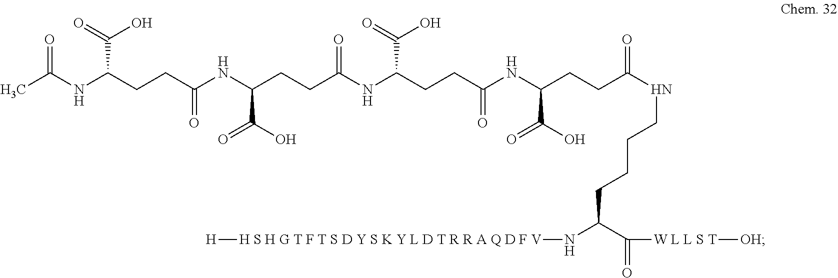
N^{ε24}-[(4S)-4-[[(4S)-4-[[(4S)-4-[[(4S)-4-amino-4-carboxybutanoyl]amino]-4-carboxybutanoyl]amino]-4-carboxybutanoyl]amino]-4-carboxybutanoyl]-[His3,Thr16,Lys24,Leu27,Ser28]-Glucagon 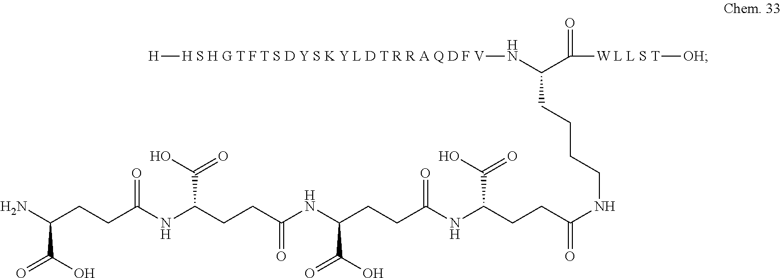
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Trp16, Lys24,Leu27,Ser28]-Glucagon 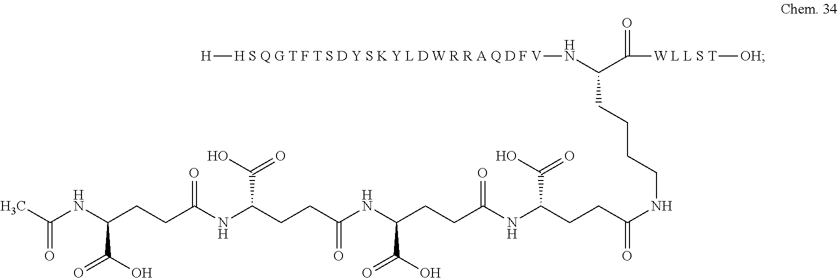
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Phe16,Lys24,Leu27,Ser28]-Glucagon 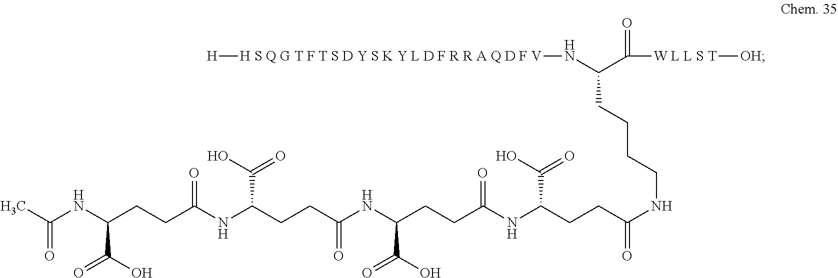
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Ile16, Lys24,Leu27,Ser28]-Glucagon 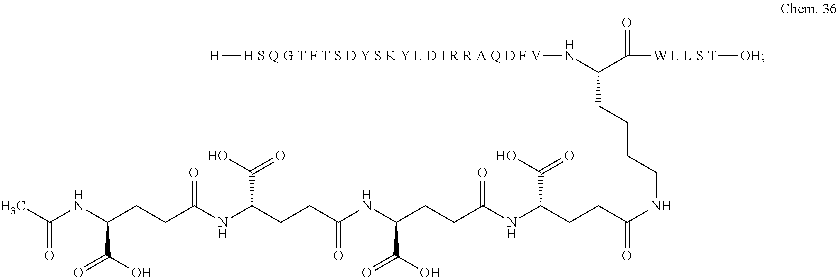
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Tyr16,Lys24,Leu27,Ser28]-Glucagon 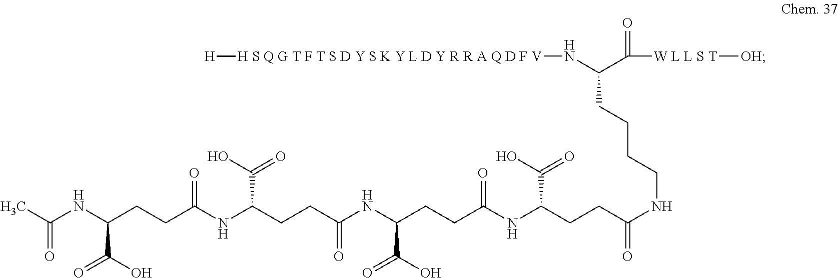
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Leu16,Lys24,Leu27,Ser28]-Glucagon 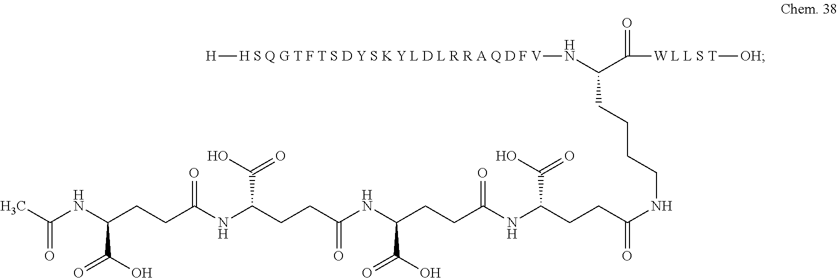
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Arg12,Leu16,Lys24,Leu27,Ser28]-Glucagon 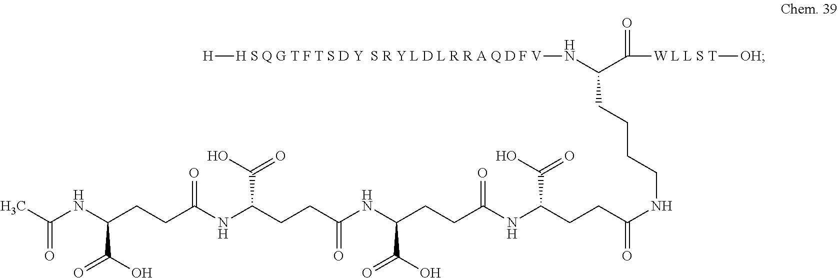
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Arg12,Leu16,Lys24,Leu27,Ser28]-Glucagonyl-Pro 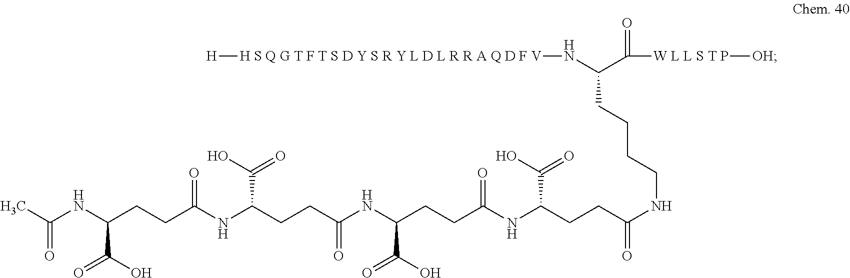
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Thr²,Leu¹⁶,Lys²⁴,Leu²⁷,Ser²⁸]-Glucagon 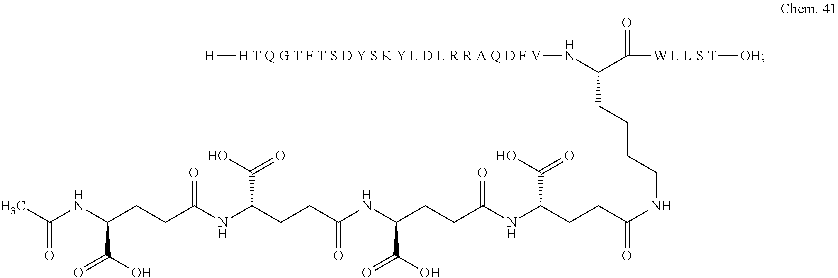
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Ala²,Leu¹⁶,Lys²⁴,Leu²⁷,Ser²⁸]-Glucagon 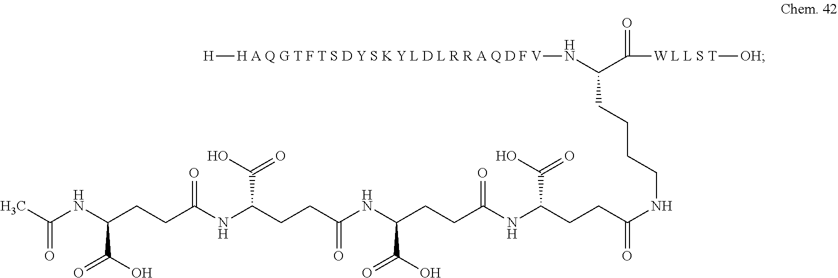
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Val¹⁰,Leu¹⁶,Lys²⁴,Leu²⁷,Ser²⁸]-Glucagon 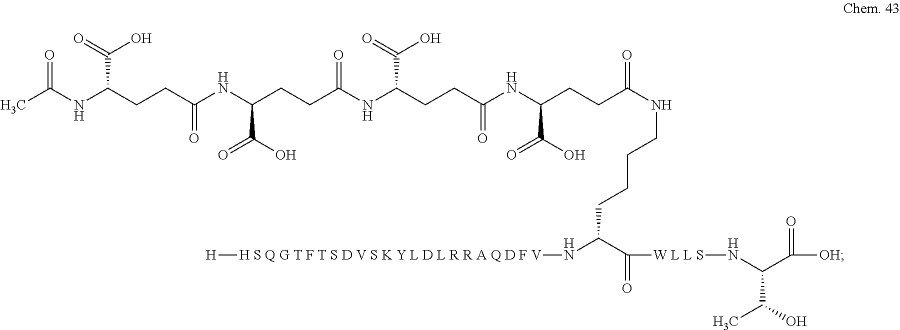
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Val¹⁰,Glu¹⁵,Lys²⁴,Leu²⁷,Ser²⁸]-Glucagon 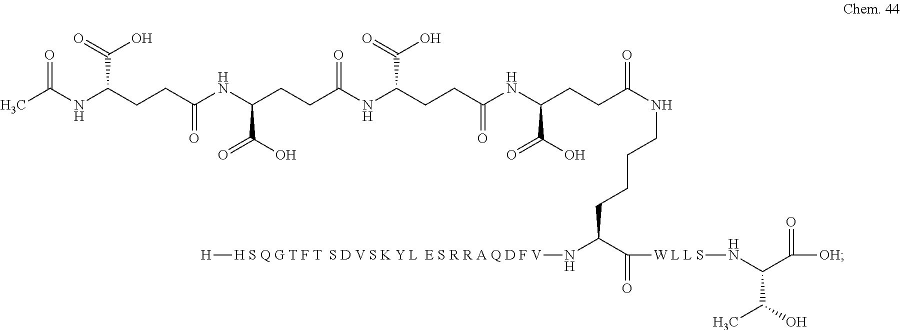
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Val¹⁰,Glu¹⁵,Glu²¹,Lys²⁴,Leu²⁷,Ser²⁸]-Glucagon 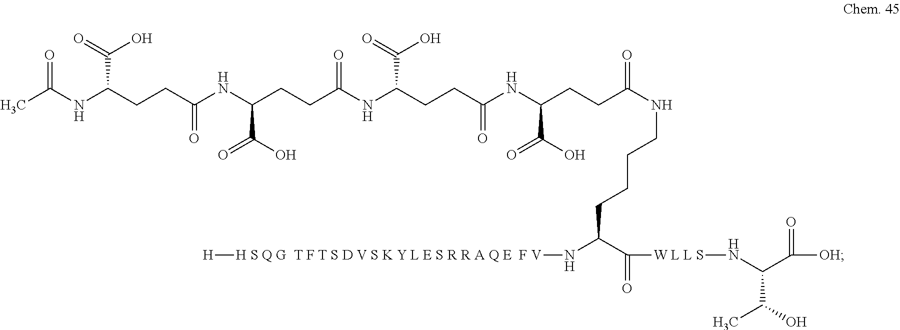
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Gly²,His³,Val¹⁶,Ala²⁰,Glu²¹,Lys²⁴,Leu²⁷,Ser²⁸]-Glucagon 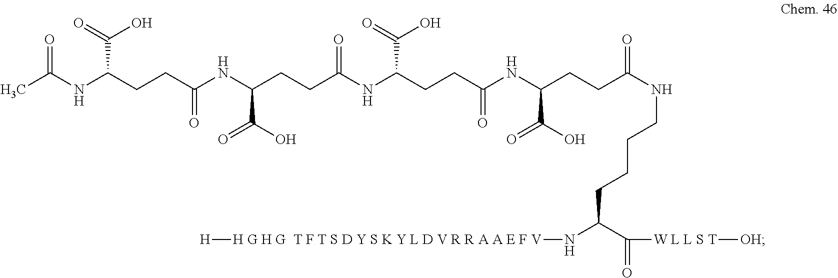
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Ala²,His³,Val¹⁶,Ala²⁰,Glu²¹,Lys²⁴,Leu²⁷,Ser²⁸]-Glucagon 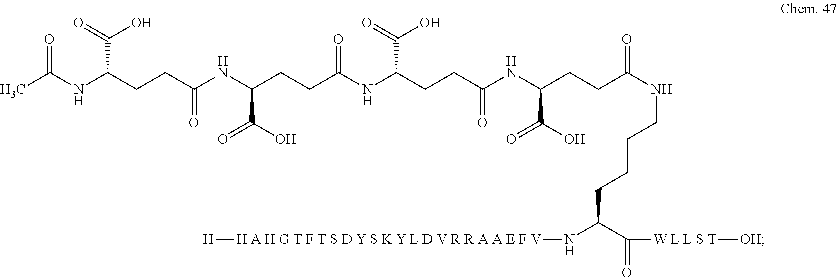
N^{ε24}-[(4S)-4 [[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Aib²,His³,Val¹⁶,Ala²⁰,Glu²¹,Lys²⁴,Leu²⁷,Ser²⁸]-Glucagon 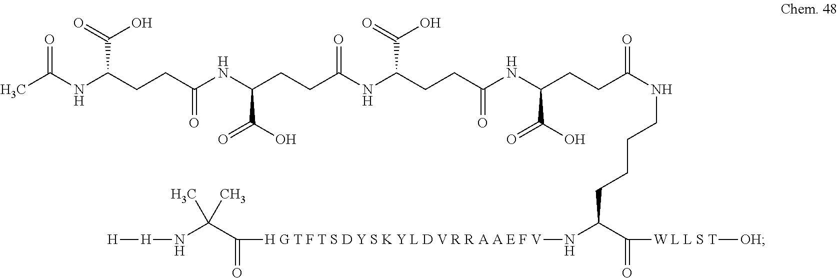
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Gly²,Val¹⁶,Ala²⁰,Glu²¹,Lys²⁴,Leu²⁷,Ser²⁸]-Glucagon 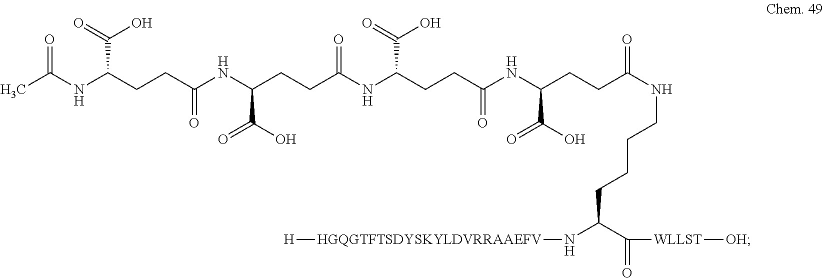
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Gly²,His³,Val¹⁰,Val¹⁶,Ala²⁰,Glu²¹,Lys²⁴,Leu²⁷,Ser²⁸]-Glucagon 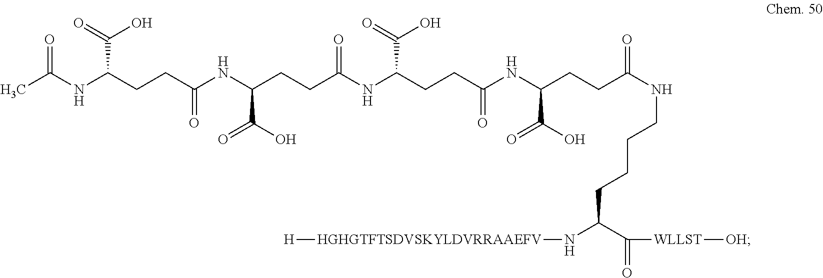
N^{ε}([Leu²⁷,Ser²⁸]-Glucagonyl)-N [(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]Lys 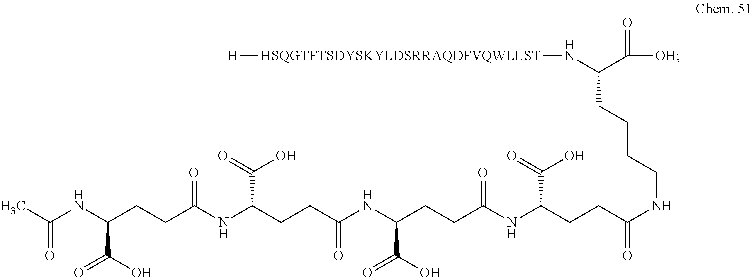
N^{ε29}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Leu²⁷,Ser²⁸,Lys²⁹]-Glucagon 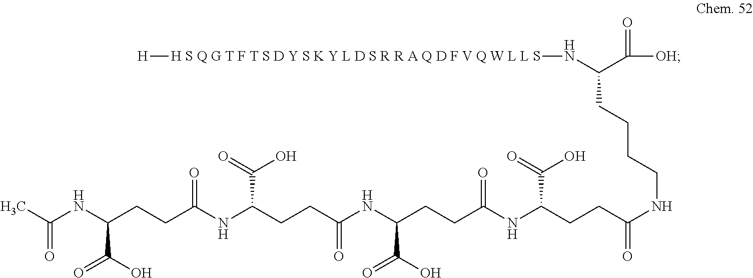
N^{ε28}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Leu²⁷,Lys²⁸]-Glucagon 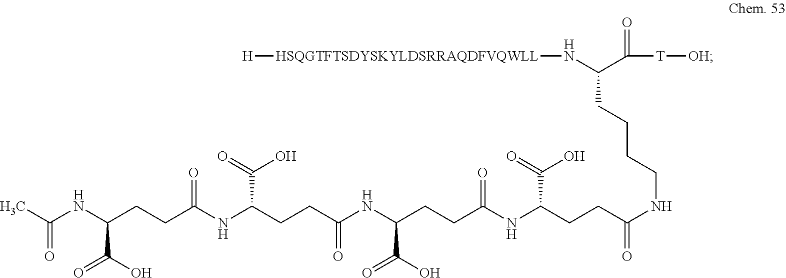
N^{ε20}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Lys²⁰,Leu²⁷,Ser²⁸]-Glucagon 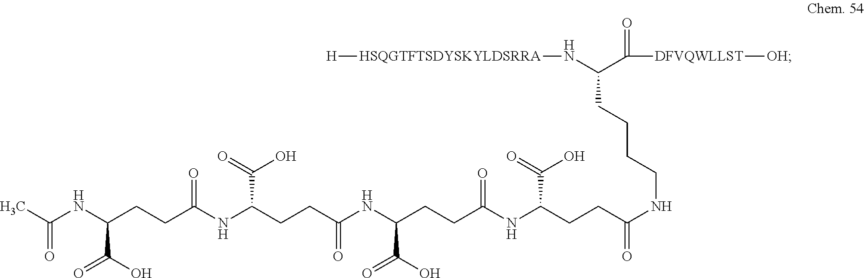
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Gly²,Leu¹⁶,Lys²⁴,Leu²⁷,Ser²⁸]-Glucagon 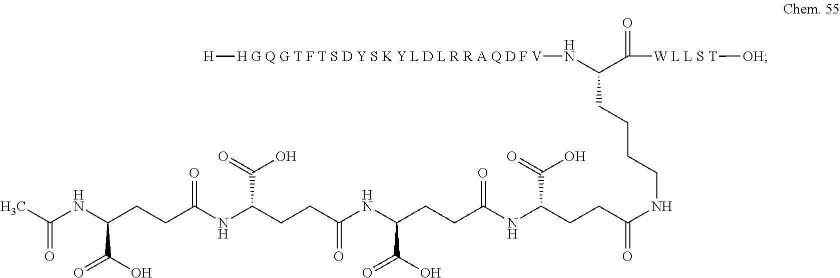
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Arg¹²,Leu¹⁶,Ala²⁰,Lys²⁴,Leu²⁷,Ser²⁸]-Glucagon 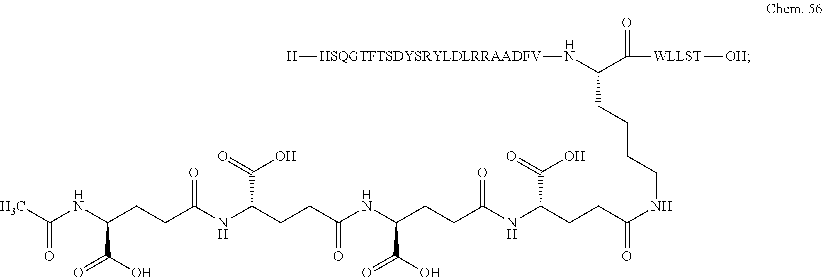
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Arg¹²,Leu¹⁶,Glu²⁰,Lys²⁴,Leu²⁷,Ser²⁸]-Glucagon 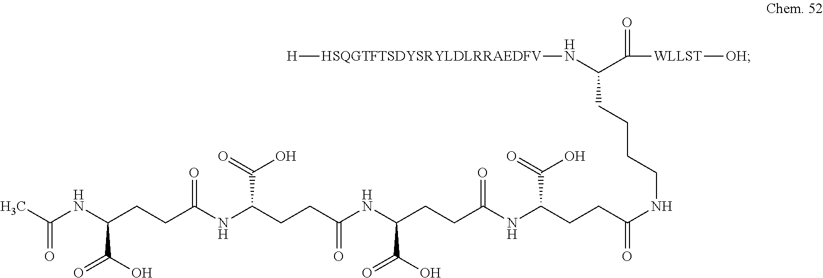
N^{ε24}-[(2S)-6-[[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]amino]-2-aminohexanoyl]-[Lys24,Leu27]-Glucagon 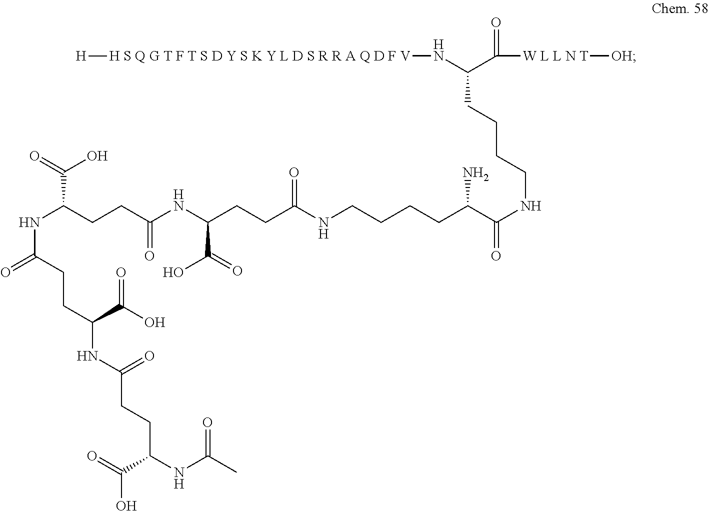
N^{ε16}-[(2S)-6-[[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]amino]-2-aminohexanoyl]-[Lys¹⁶,Ala¹⁸,Leu²⁷,Ser²⁸]-Glucagon amide 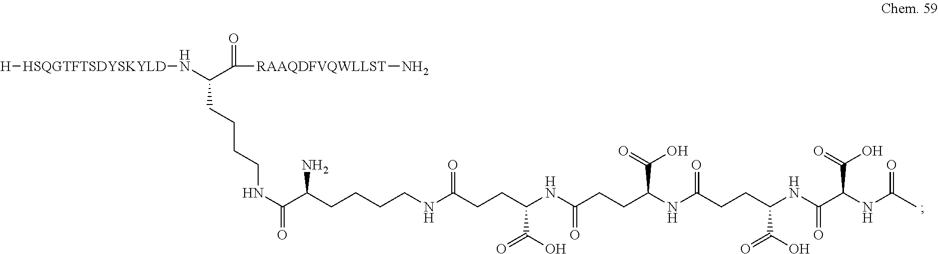
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Ala²,His³,Leu¹⁶,Lys²⁴,Leu²⁷,Ser²⁸]-Glucagon 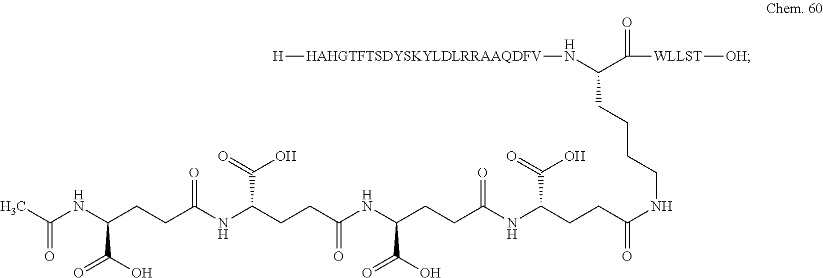
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Ala²,Val¹⁰,Leu¹⁶,Ala²⁰,Lys²⁴,Leu²⁷,Ser²⁸]-Glucagon 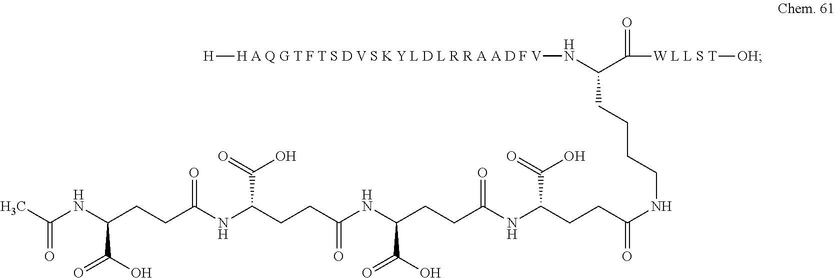
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Ala²,His³,Val¹⁰,Leu¹⁶,Ala²⁰,Lys²⁴,Leu²⁷,Ser²⁸]-Glucagon 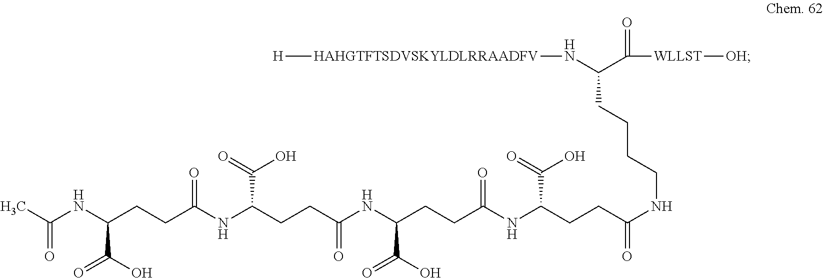
N^{ε24}-[(4S)-4-carboxy-4-[[(4S)-4-carboxy-4-[[(4S)-4-carboxy-4-[[(4S)-4-carboxy-4-(propanoylamino)butanoyl]amino]butanoyl]amino]butanoyl]amino]butanoyl]-[Arg¹²,Leu¹⁶,Ala²⁰,Lys²⁴,Leu²⁷,Ser²⁸]-Glucagon 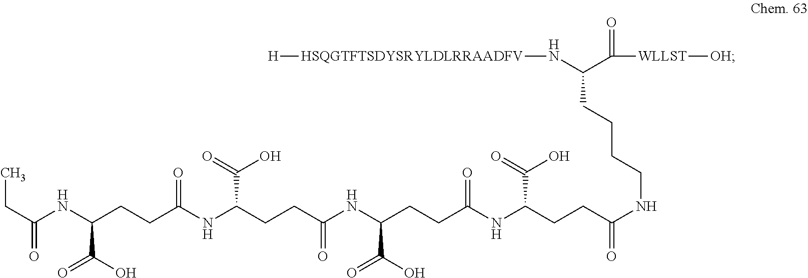
N^{ε24}-[(4S)-4-carboxy-4-[[(4S)-4-carboxy-4-[[(4S)-4-carboxy-4-[[(4S)-4-carboxy-4-(2-methylpropanoylamino)butanoyl]amino]butanoyl]amino]butanoyl]amino]butanoyl]-[Arg12,Leu16,Ala20,Lys24,Leu27,Ser28]-Glucagon 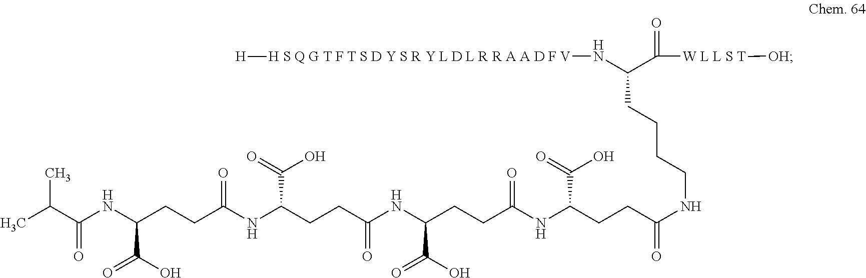
N^{ε24}-[(4S)-4-[[(4S)-4-[[(4S)-4-[[(4S)-4-(butanoylamino)-4-carboxybutanoyl]amino]-4-carboxybutanoyl]amino]-4-carboxybutanoyl]amino]-4-carboxybutanoyl]-[Arg¹²,Leu¹⁶,Ala²⁰,Lys²⁴,Leu²⁷,Ser²⁸]-Glucagon 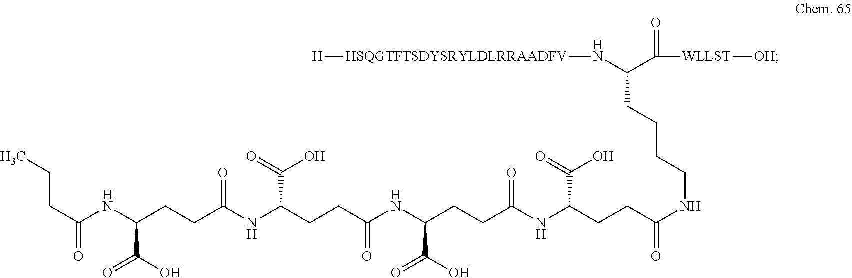
N^{ε24}-[(4S)-4-carboxy-4-[[(4S)-4-carboxy-4-[[(4S)-4-carboxy-4-[[(4S)-4-carboxy-4-(3-carboxypropanoylamino)butanoyl]amino]butanoyl]amino]butanoyl]amino]butanoyl]-[Arg¹²,Leu¹⁶,Ala²⁰,Lys²⁴,Leu²⁷,Ser²⁸]-Glucagon 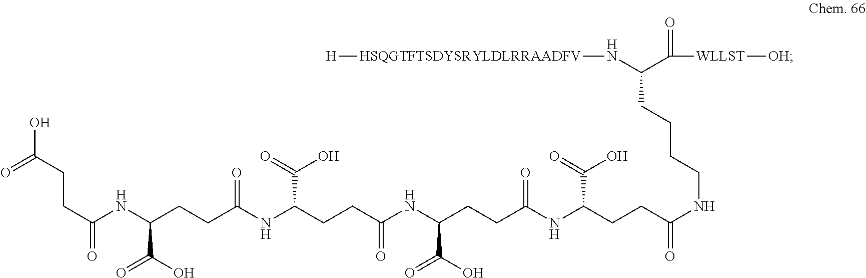
N^{ε24}-[(4S)-4-carboxy-4-[[(4S)-4-carboxy-4-[[(4S)-4-carboxy-4-[[(4S)-4-carboxy-4-(pentanoylamino)butanoyl]amino]butanoyl]amino]butanoyl]amino]butanoyl]-[Arg¹²,Leu¹⁶,Ala²⁰,Lys²⁴,Leu²⁷,Ser²⁸]-Glucagon 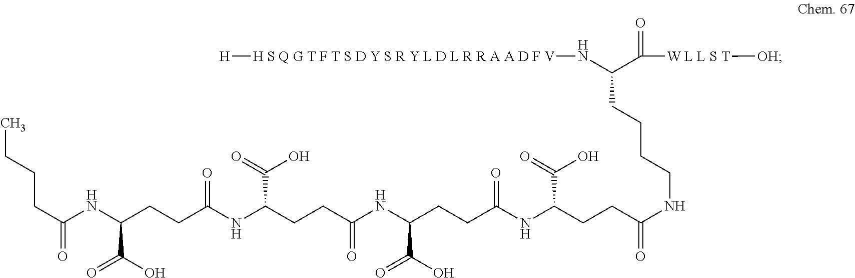
N^{ε24}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Lys16,Leu27,Ser28]-Glucagon 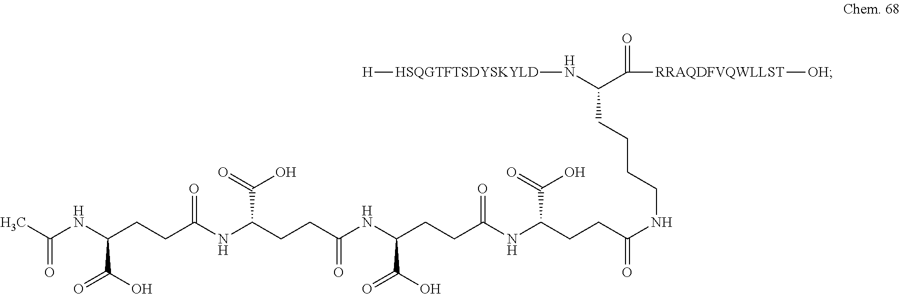
and
N^{ε21}-[(4S)-4-[[(4S)-4-[[(4S)-4-[[(4S)-4-acetamido-4-carboxybutanoyl]amino]-4-carboxybutanoyl]amino]-4-carboxybutanoyl]amino]-4-carboxybutanoyl]-[Lys21,Leu27,Ser28]-Glucagon

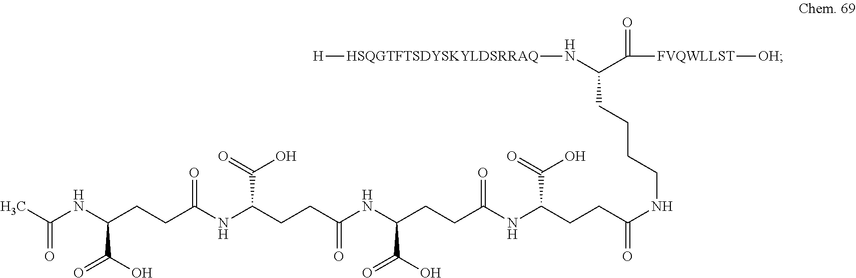

8. A pharmaceutical composition comprising a glucagon peptide according to claim 1.

9. The pharmaceutical composition according to claim 8, further comprising an additional therapeutically active compound or substance.

10. The pharmaceutical composition according to claim 8, which is suited for parenteral administration.

11. A method of treating obesity comprising administering to a patient in need thereof, an effective amount of a glucagon peptide derivative according to claim 1.

12. A method for treating of hyperglycemia comprising administering to a patient in need thereof, an effective amount of a glucagon peptide derivative according to claim 1.

13. A method for treating type 2 diabetes comprising administering to a patient in need thereof, an effective amount of a glucagon peptide derivative according to claim 1.

14. A method for treating impaired glucose tolerance comprising administering to a patient in need thereof, an effective amount of a glucagon peptide derivative according to claim 1.

15. A method for treating type 1 diabetes comprising administering to a patient in need thereof, an effective amount of a glucagon peptide derivative according to claim 1.


SEQUENCE LISTING (1 sequence)

SEQ ID NO: 1 - protein, 29 aa, Homo sapiens
HSQGTFTSDYSKYLDSRRAQDFVQWLMNT